(19) United States
(12) Patent Application Publication
(10) Pub. No.: US 2024/0229145 A9
(43) Pub. Date: Jul. 11, 2024

(54) APPARATUS AND METHOD FOR EARLY CANCER DETECTION AND CANCER PROGNOSIS USING A NANOSENSOR WITH RAMAN SPECTROSCOPY

(71) Applicants: Bo Tan, Mississauga (CA); Krishnan Venkatakrishnan, Mississauga (CA); Swarna Ganesh, Toronto (CA); Haldavnekar Rupa Ravindra, Oakville (CA)

(72) Inventors: Bo Tan, Mississauga (CA); Krishnan Venkatakrishnan, Mississauga (CA); Swarna Ganesh, Toronto (CA); Haldavnekar Rupa Ravindra, Oakville (CA)

(21) Appl. No.: 18/277,686

(22) PCT Filed: Feb. 17, 2022
(86) PCT No.: PCT/CA2022/050232

§ 371 Date: Aug. 17, 2023

Related U.S. Application Data
(60) Provisional application No. 63/150,566, filed on Feb. 17, 2021.

(51) Int. Cl. C12Q 1/6886; C12Q 1/6825; G01N 33/574; G01N 33/68; G16H 10/40

(52) U.S. Cl. CPC C12Q 1/6886; C12Q 1/6825; G01N 33/57488; G01N 33/6848; G16H 10/40; C12Q 2600/154; C12Q 2600/156; G01N 2560/00

(57) ABSTRACT

Embodiments of a computing device and methods of providing a cancer assessment for a patient are described. The method involves isolating a volume of a fluid from a fluid sample of the patient, the volume of fluid including at least one biomarker; adding at least a portion of the volume of fluid to a nanosensor comprising nanoparticles configured to capture the at least one biomarker and amplify signals emitted by the at least one biomarker during Raman spectroscopy; performing Raman spectroscopy on the volume of fluid on the nanosensor to produce a sample Raman spectrum having amplified signals indicating the presence of the at least one biomarker on the nanosensor; processing the sample Raman spectrum using data from template Raman spectra from known cancer samples; and based on the detected one or more cancer characteristics, providing the cancer assessment of the patient.

### FIELD

This disclosure relates generally to cancer detection and prognosis, and more specifically to apparatus and methods for early cancer detection and prognosis using a nanosensor with Raman spectroscopy.

### BACKGROUND

The cancer burden has continued to grow globally with there being an estimated 18 million cancer cases and 9.6 million deaths caused by cancer in 2018. The majority of cancers can be cured when the disease gets diagnosed while it is still confined to the organ of origin. Therefore, rapid screening of onset of cancer is a key to cure cancer.

Unfortunately, many types of cancers only get diagnosed at advanced stages because the diagnostic intervention is often related to symptoms. Therefore, cancer screening, a strategy for early cancer intervention, needs to be employed to detect disease markers before the presentation of noticeable symptoms.

Many cancers have a better prognosis if they are diagnosed early on, which has led to development of many cancer screening protocols involving efforts to detect cancer in symptomatic as well as occult cancers. The existing successful screening technologies involve identifying precursor lesions, endoscopic biopsies, mammography, colonoscopy and/or cervical cytology. But imaging-based screening suffers from sensitivity and the biopsy-based tests have limitations in assessment of cancer development, prognosis as well as genotyping. This happens because of tumor heterogeneity and cancer evolution. Also, accessing tumors in very early stage is clinically challenging, morbid and expensive. The trauma, challenges and invasiveness of accessing the tumor in the initial stages can be eliminated by alternative blood-based screening technology. The idea of a simple blood test to identify malignant changes, have fueled interest in the constant search of blood-based rapid cancer screening markers and methodologies.

Liquid biopsy, such as blood, urine, stool, mucus or other body fluids, have been used widely in medical diagnosis and treatment response assessments. For instance, all types of cancers, Alzheimer's and various infectious diseases and treatment can be analysed with liquid biopsy. For some medical conditions, liquid biopsy contains far less biomarkers than tissue biopsies. This is particularly true for various disease originated from deep body organs or brain, for instance, ovarian cancer, brain cancer and Alzheimer's disease. Only very small amounts of biomarker molecules will be released to the systems of a human body from these hard-to-reach organs. Therefore, the applicability of liquid biopsy to disease diagnosis and treatment assessment is dependent on the sensitivity of detection device. The detection device should have a sensitive enough Limit of Detection (LoD) to report the presence of biomarkers, even at very low concentration. Trace level detection is necessary for any sensor to be effective for rapid screening at the onset of cancer because of the low availability of the biomarkers in very early stages of tumorigenesis.

The advance of using liquid biopsy is its accessibility, low risk, non-invasiveness, which allows for repeated sample collection. Liquid biopsy is often considered as the key to early diagnosis and screening of disease. Unfortunately, limited by LoD, many liquid-biopsy-based tests often times show low sensitivity or specificity and thus have to be used as a secondary and supplementary tool for diagnosis. For instance, recent research has shown that ctDNA and CTC from blood can identify early stage and asymptotic cancer. However, due to the unsatisfactory accuracy, it has to be used together with image detection for a confirmed diagnosis. Since the imaging techniques cannot detect tumours that are less than about 7-millimetre size, the purpose of using a blood test for the early diagnosis of onset tumour is completely lost using conventional techniques. In clinical settings, ctDNA and CTC assays are only used for therapy monitoring.

Accordingly, there is a need for new liquid-based tests for the diagnosis of cancer.

### SUMMARY

In accordance with one broad aspect, at least one example embodiment described in accordance with the teachings herein provides a method of providing a cancer assessment for a patient, the method comprising: isolating a volume of a fluid from a fluid sample of the patient, the volume of fluid including at least one biomarker; adding at least a portion of the volume of fluid to a nanosensor, the nanosensor comprising nanoparticles configured to capture the at least one biomarker and amplify signals emitted by the at least one biomarker during Raman spectroscopy; performing Raman spectroscopy on the volume of fluid on the nanosensor to produce a sample Raman spectrum, the sample Raman spectrum having amplified signals indicating the presence of the at least one biomarker on the nanosensor; processing the sample Raman spectrum using data from template Raman spectra from known cancer samples having cancer characteristics to detect whether the sample comprises one or more of the cancer characteristics; and based on the detected one or more cancer characteristics, providing the cancer assessment of the patient.

In at least one embodiment, the one or more cancer characteristics of the sample are detected based on determining which correlation values obtained by correlating the amplified signals of the sample Raman spectrum to template Raman spectra from the known cancer samples having the cancer characteristics are larger than a correlation threshold.

In at least one embodiment, the one or more cancer characteristics of the sample are detected by: performing feature extraction on the Raman sample spectral data to extract feature values; performing classification by applying the feature values to at least one set of classification models determined for the at least one biomarker to detect the one or more cancer characteristics; and providing the cancer assessment by incorporating each of the detected cancer characteristics, wherein the classification models are determined using the template Raman spectra from the known cancer samples.

In at least one embodiment, the feature extraction is performed using Principal Component Analysis, Multivariate Curve Resolution Analysis or a combination thereof.

In at least one embodiment, the feature extraction is performed using Principal Component Analysis, Multivariate Curve Resolution Analysis or a combination thereof.

In at least one embodiment, the classification model is one of Partial Least Squares Discriminant Analysis (PLSDA), Support Vector Machine Discriminant Analysis (SVMDA) and Artificial Neural Network analysis (ANN), TSNE, Random Forest classification.

In at least one embodiment, the cancer characteristic is a cancer type, a cancer stage, cancer progression, cancer metastasis, cancer potential for metastasis, prediction of a benign or malignant tumor, prediction of tumor location, or a combination thereof.

In at least one embodiment, the biomarker is extracellular vesicles.

In at least one embodiment, the biomarker is extracellular vesicles associated with circulating cancer initiating cells (CICs) or cancer stem cells.

In at least one embodiment, the biomarker is cell-free nucleic acid associated with cancer initiating cells (CICs) or cancer stem cells.

In at least one embodiment, the cell-free nucleic acid is as cell free DNA.

In at least one embodiment, the cell free DNA is molecularly modified by one of methylation, oxidation and acetylation.

In at least one embodiment, the biomarker is immune cells.

In at least one embodiment, the biomarker is one or more of T− cells, NK cells and myeloid derived suppressor cells.

In at least one embodiment, the biomarker is one or more of CD 4+ T cells, NK cells and β cells.

In at least one embodiment, the biomarker is serum

In at least one embodiment, the fluid is obtained by density gradient centrifugation.

In at least one embodiment, the volume of fluid is about 10 μL or more.

In at least one embodiment, the fluid is blood plasma and the volume of the blood plasma is about 10 μL or more.

In at least one embodiment, the fluid is buffy coat and the volume of the buffy coat is about 10 μL or more.

In at least one embodiment, after adding the fluid to the nanosensor, the fluid remains on the nanosensor for an incubation period.

In at least one embodiment, the incubation period is in a range of about 1 minute to about 2 minutes.

In at least one embodiment, the method includes providing the cancer assessment includes providing a type of the cancer, a location of the cancer, a stage of the cancer, a metastatic potential of the cancer, a therapy efficacy of the cancer or a monitoring of minimal residual disease.

In at least one embodiment, providing the cancer assessment includes early cancer diagnosis.

In at least one embodiment, providing the cancer assessment includes determining whether a tumor is benign or malignant.

In at least one embodiment, providing the cancer assessment includes, when the tumor is benign, determining weather the tumor has potential for malignancy.

In at least one embodiment, providing the cancer assessment includes determining whether a tumor is primary or metastatic.

In at least one embodiment, providing the cancer assessment includes determining whether a primary tumor has potential for metastasis.

In at least one embodiment, providing the cancer assessment includes determining a progression of the cancer.

In at least one embodiment, providing the cancer assessment includes determining a nodal metastasis of the cancer.

In at least one embodiment, providing the cancer assessment includes determining a clinical metastasis of the cancer.

In at least one embodiment, providing the cancer assessment includes determining a stage and/or grade of the cancer.

In at least one embodiment, providing the cancer assessment includes a prediction of patient survival.

In at least one embodiment, providing the cancer assessment includes providing a prognosis for the patient.

In at least one embodiment, providing the cancer assessment includes providing an early diagnosis of cancer.

In at least one embodiment, providing the cancer assessment includes providing the early diagnosis of hard to detect cancers including brain cancer, ovarian cancer, kidney cancer, pancreatic cancer, liver cancer, lung cancer, or glastrointerstinal cancer.

In at least one embodiment, providing the cancer assessment includes determining a presence of an aggressive brain cancer including glioblastoma.

In at least one embodiment, providing the cancer assessment includes providing a location of the tumor.

In at least one embodiment, providing the cancer assessment includes determining a metastatic state of cancer to brain from a cancer site, the cancer site including lung tissue, breast tissue, colon tissue, kidney tissue, thyroid tissue and skin tissue.

In at least one embodiment, determining the metastatic state is by risk assessment based on a molecular phenotype of the tumor, the molecular phenotype including human epidermal growth factor receptor 2 (HER 2), epidermal growth factor receptor (EGFR) and/or isocitrate dehydrogenase (IDH).

In at least one embodiment, determining the metastatic state of cancer to brain from a cancer site includes determining the metastatic state of breast cancer based on a molecular phenotype of the tumor.

In at least one embodiment, the molecular phenotype of the tumor is HER2 positive or HER 2 negative.

In at least one embodiment, determining the metastatic state of cancer to brain from a cancer site includes determining the metastatic state of lung cancer based on a molecular phenotype of the tumor.

In at least one embodiment, the molecular phenotype of the tumor is EGFR.

In at least one embodiment, providing the cancer assessment includes determining a metastatic state of cancer to localised metastasis or widespread from primary cancer sites.

In at least one embodiment, providing the cancer assessment includes determining presence a gynaecological cancer, the gynaecological cancer being one of ovarian cancer, cervical cancer, or uterine cancer.

In at least one embodiment, providing the cancer assessment includes monitoring cancer recurrence during or after therapy, the therapy including radiation therapy, immunotherapy and/or chemotherapy.

In at least one embodiment, providing the cancer assessment includes monitoring cancer recurrence after surgery.

In at least one embodiment, providing the cancer assessment includes determining a presence of minimal residual disease.

In at least one embodiment, the sample Raman spectrum includes amplified signals indicating the presence of a second biomarker on the nanosensor and the method further comprises: performing further data processing on the sample Raman spectrum to compare the second amplified signals to a second template Raman spectrum to determine a correlation between the sample Raman spectrum and the second template Raman spectrum, the template Raman spectrum being of a known cancer characteristic, and based on both the correlation between the sample Raman spectrum and the template Raman spectrum and the second correlation between the sample Raman spectrum and the second template Raman spectrum, providing the diagnosis of the cancer in the patient.

In accordance with another broad aspect, at least one example embodiment described in accordance with the teachings herein provides a computing device for providing a cancer assessment for a patient for a sample that is a volume of a fluid sample from the patient that includes at least one biomarker; wherein the computing device comprises: a data store comprising program instructions for obtaining Raman spectral data of the sample and performing cancer assessment of the sample using the sample Raman spectral data; and a processing unit that is operatively coupled to the data store and when executing the program instructions is configured to: acquire sample Raman spectral data from the fluid sample where the Raman spectral is obtained after adding at least a portion of the volume of the fluid sample to a nanosensor that comprises nanoparticles configured to capture the at least one biomarker, the sample Raman spectral data having amplified signals indicating the presence of the at least one biomarker on the nanosensor; process the sample Raman spectral data using data from template Raman spectra from known cancer samples having cancer characteristics to detect whether the sample comprises one or more of the cancer characteristics; and provide the cancer assessment of the patient based on the detected one or more cancer characteristics.

In at least one embodiment, the processing unit is further configured to perform any of the steps of the methods described in accordance with the teachings herein.

These and other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1A is a block diagram showing steps of a method of cancer detection, according to at least one example embodiment described herein.

FIG. 1B is a block diagram of the hardware components used in obtaining Raman spectra of biomarkers on nanosensors during the method of FIG. 1A, according to at least one example embodiment described herein.

FIG. 1C is a block diagram of an example embodiment of a computing device and software that may be used with the hardware setup of FIG. 1B.

FIG. 1D is a flowchart of a method for performing a cancer assessment on a patient sample using Raman spectra obtained therefrom that may be performed by software of the computing device of FIG. 1C, according to at least one example embodiment described herein.

FIG. 1E is a flowchart of a method for performing training to obtain models that may be used by the software of the computing device of FIG. 1C in detecting various conditions from the patient sample.

FIG. 2 shows three-dimensional architectures of three Graphene Oxide Quantum Sensors (GOQS) and graphs providing their mean particle size.

FIG. 3A is a collection of High Resolution Scanning Electron Microscopy (HRSEM) images that demonstrate spherical morphology with intertwined network arrangements.

FIG. 3B is a collection of Transmission Electron Microscopy (TEM) images that reveal extremely small size of the nanoprobes.

FIG. 3C is a collection of High Resolution Transmission Electron Microscopy (HRTEM) images that demonstrate crystalline nature and surface defects of the nanoprobes.

FIG. 3D is a collection of graphs showing mean particle size of the nanoprobes varies between 6 nm and 17 nm.

FIGS. 3E and 3F are XPS images that reveal rutile phase and oxygen vacancy defects.

FIG. 3G is a collection of Raman spectra that confirm formation of titanium oxide in the nanoprobes.

FIG. 4 shows Raman spectra of circulating extracellular vesicles at various concentrations where the limit of detection was up to 10 circulating extracellular vesicles in 10 μL solution and peaks that were assigned to nucleic acids, proteins and lipids were clearly visible at ultra-low concentration.

FIG. 5A is a schematic representation of circulating extracellular vesicles captured on a nanosensor along with Field Emission Scanning Electron Microscope (FESEM) images of circulating extracellular vesicles on the nanosensor.

FIG. 5B shows SERS spectra from circulating extracellular vesicles derived from breast, lung and colorectal cancer cells and Cancer Stem Cells (CSCs).

FIG. 6A shows FESEM images of circulating extracellular vesicles trapped on a cancer nanosensor, which is the same nanosensor used in FIG. 5A.

FIG. 6B shows an ability of the nanosensor of FIG. 6B to identify variation in the nanosomal contents of the circulating extracellular vesicles of lung fibroblast cells, lung cancer cells and lung CSCs.

FIG. 6C shows Raman spectra of circulating extracellular vesicles at various concentrations.

FIG. 7A shows TEM images of circulating extracellular vesicles of breast cancer cells and breast CSCs.

FIG. 7B shows SERS profiles of breast cancer cells and breast CSCs demonstrating a variation in Raman assignments.

FIG. 7C shows a (i) Heat map of Raman peaks demonstrated variation in the nucleic acids, proteins as well as lipids, (v) a Bee swarm plot of functional component in Multivariate Curve Resolution analysis demonstrated variation between extracellular vesicles of cancer cells vs CSC extracellular vesicles (ii) PC1 (iii) PC2s demonstrated statistical significance and (iv) distinct clustering.

FIG. 7D shows a bee swarm plot of functional components in Multivariate Curve Resolution analysis that demonstrates significant variation between Circulating extracellular vesicles of cancer cells vs Circulating extracellular vesicles of CSCs.

FIG. 7E shows scatter plots of cancer extracellular vesicles and extracellular vesicles of CSCs for various principle components (PCs).

FIG. 8A shows TEM images of circulating extracellular vesicles.

FIG. 8B shows SERS profiles of lung cancer cell extracellular vesicles and lung CSCs demonstrating a variation in Raman assignments.

FIG. 8C shows (i) a heat map of Raman peaks demonstrated variation in the nucleic acids, proteins as well as lipids (v) Bee swarm plot of functional component in Multivariate Curve Resolution analysis demonstrated variation between extracellular vesicles of cancer cells vs CSC extracellular vesicles (ii) PC1 (iii) PC2s demonstrated statistical significance and (iv) distinct clustering.

FIG. 8D is a bee swarm plot of functional component in Multivariate Curve Resolution analysis and shows a demonstrated variation between extracellular vesicles of cancer cells and CSC extracellular vesicles.

FIG. 8E shows scatter plots of cancer extracellular vesicles of CSCs for various principle components (PCs).

FIG. 9A shows TEM images of circulating extracellular vesicles.

FIG. 9B shows a SERS profile of colorectal cancer cells and colorectal CSCs and their demonstrated variation in Raman assignments.

FIG. 9C shows (i) a Heat map of Raman peaks demonstrated variation in the nucleic acids, proteins as well as lipids (v) Bee swarm plot of functional component in Multivariate Curve Resolution analysis demonstrated variation between extracellular vesicles of cancer cells vs CSC extracellular vesicles (ii) PC1 (iii) PC2s demonstrated statistical significance and (iv) distinct clustering.

FIG. 9D is a bee swarm plot of functional components in Multivariate Curve Resolution analysis and shows demonstrated variation between circulating extracellular vesicles of cancer cells versus CSC extracellular vesicles.

FIG. 9E show scatter plots of cancer extracellular vesicles of CSCs for various principle components.

FIG. 10A shows a principal component analysis including three scatter plots that indicate a demonstrated ability to discriminate between circulating extracellular vesicles of cancer initiating breast, colorectal and lung cancer cells and a heat map of signature Raman assignments demonstrating distinct variation in proteins, lipids, nucleic acid contents of the circulating extracellular vesicles.

FIG. 10B shows a partial least square discriminant analysis (PLSDA) classification that demonstrates the ability to classify between the three types of cancers with the Raman signatures derived from the circulating extracellular vesicles of CSCs.

FIG. 11A shows a comparison of Raman Spectra of plasma without enrichment or extracellular vesicles isolation and Raman Spectra of extracellular vesicles isolated from plasma.

FIG. 11B shows heat maps of signature Raman Peaks from extracellular vesicles isolated from plasma and Raman spectra directly obtained from plasma demonstrating similar biomolecular features.

FIG. 11C shows an MCR analysis for Raman Spectra of extracellular vesicles isolated from plasma and Raman spectra directly obtained from plasma demonstrating correlation.

FIG. 12 shows a prediction of localization of tumor with clinical plasma samples—principal component analysis that demonstrated clustering of clinical samples with training data (extracellular vesicles of cancer inviting cells) showing similar properties. This hierarchical model was built for prediction of tumor location with 91% accuracy.

FIG. 13 shows a mapping of circulating DNA structure for cancer (MCSC).

FIG. 14A shows variation in Raman profile of Cancer DNA and CSC DNA—(i) Raman spectra of breast cancer DNA and breast CSC DNA; (ii) Colorectal cancer DNA and Colorectal CSC DNA; and (iii) Lung Cancer DNA and Lung CSC DNA demonstrated variation in the peak intensity.

FIG. 14B shows a heatmap of major DNA peaks showing variation in the presence of DNA components in CSC DNA as compared to cancer cell DNA.

FIG. 14C shows a scatter plot of PC1 Vs PC2 that demonstrates negative correlation.

FIG. 15A shows an analysis of similarity between cancer cell DNA, CSC DNA and tumor DNA.

FIG. 15B shows a grade wise analysis that demonstrates higher correlation between tumor DNA and CSC DNA.

FIG. 16A shows a multiresolution curve analysis and Raman spectra demonstrating substantial similarity between cfDNA and tumorDNA for breast cancer.

FIG. 16B shows a multiresolution curve analysis and Raman spectra demonstrating substantial similarity between cfDNA and tumorDNA for breast cancer.

FIG. 16C shows a multiresolution curve analysis and Raman spectra demonstrating substantial similarity between cfDNA and tumorDNA for lung cancer.

FIG. 17 shows a schematic illustration of quantum superstructure-assisted liquid profiling by MCSC of cfDNA-based diagnosis of hard to detect cancers with a simple machine learning training model.

FIG. 18A shows a schematic illustration of quantum probe-assisted feature extraction from training data.

FIG. 18B shows a schematic illustration of detection of presence of cancer directly from blood plasma without cfDNA isolation, and (i) discrimination between cancer and non-cancer DNA (training data); and (ii) identification of cancer directly from plasma with classification sensitivity.

FIG. 19 shows identification of tissue of origin directly from raw blood plasm, collection of spectroscopic data from cell culture DNA of cancer stem cells and tumor DNA for identification of tissue of origin, identification of lung cancer (sensitivity 83% specificity 96%), breast cancer (sensitivity 86% specificity 92%) and colorectal cancer (sensitivity 75% specificity 92%).

FIG. 20A shows principal component analysis demonstrating clear clustering between non cancer EVs vs early and late cancer EVs.

FIG. 20B shows a heatmap of the loadings of PC1 demonstrating the ability of our methodology to discriminate between brain cancer and non-cancer.

FIG. 20C shows a PCA demonstrating clear clustering between early brain cancer vs advanced brain cancer.

FIG. 20D shows a heatmap of the loadings of PC2 demonstrating the ability of the methodology to discriminate between early grade brain cancer vs advanced brain cancer.

FIG. 21A shows a Raman spectra of serum of a patient with a benign brain tumor.

FIG. 21B shows a Raman spectra of serum of a patient with a malignant brain tumor.

FIG. 21C shows distinct clustering of tumor types demonstrating ability of the methodology for clear discrimination

FIG. 21D shows ANN demonstrated 100% accuracy for prediction of the malignancy of tumor

FIG. 21E shows a confusion matrix for malignancy prediction.

FIG. 22A shows a schematic representation of the sensor with hybrid quantum hyperstructures generating preferential adsorption of methyl groups and a SERS spectra of DNA with differential methylation percentage.

FIG. 22B shows SERS peak intensity analysis to showcase the enhancement efficiency of the sensor.

FIG. 22C shows ultra-high limit of detection (Femto gram DNA concentration).

FIG. 22D shows quantification of methylation from preclinical models.

FIG. 22E shows quantification of circulating methylation.

FIG. 23 shows two graphs that provide discrimination of cfDNA methylation signature between metastatic and non-metastatic tumor samples.

FIG. 24 shows a Raman spectral comparison of extracellular vesicles derived from colorectal cancer cells and colorectal CSCs to determine the signature of metastatic and non-metastatic proteins in extracellular vesicles cargo.

FIG. 25 shows a Raman spectral comparison of extracellular vesicles derived from lung cancer cells and lung CSC to determine the signature of metastatic and non-metastatic proteins in nanosomal cargo.

FIG. 26 shows a Raman spectral comparison of extracellular vesicles derived from breast cancer cells and breast CSCs to determine the signature of metastatic and non-metastatic proteins in nanosomal cargo.

FIG. 27A shows a schematic representation of DNA adsorbed on the Quantum Hyperstructures.

FIG. 27B shows presence of metal assisted semiconductor charge transfer resonance.

FIG. 27C shows surface functionalization of quantum hyperstructures for selective adsorption of methyl groups.

FIG. 27D shows exciton quenching resulting in substantially enhanced SERS.

FIG. 27E shows charge transfer resonance showcased by the presence of photoluminescence quenching with increasing methylation percentage confirming the effective trapping of methylated DNA.

FIG. 27F shows absorption spectra to showcase the reduction in fluorescent intensity.

FIG. 27G shows Intensity of absorption at 535.8 nm demonstrated PL quenching confirming preferential adsorption of methylated DNA

FIG. 28A shows Mapping Circulating Methylation for Metastasis (MCMM) instrumental in analysis of variance associated with global methylation of cancer cells vs cancer stem cells (CSC).

FIG. 28B shows results for cancer cell, CSC and tumor DNA being analyzed for investigation of associated similarities with MCMM

FIG. 28C shows results of cancer cell, CSC, Tumor DNA and plasma being investigated to analyze associated similarity for (i) Breast cancer (ii) Lung Cancer and (iii) colorectal cancer.

FIG. 29A shows a schematic illustration for the application of MCMM for cancer diagnosis.

FIG. 29B shows a PCA analysis showing the differences between healthy, cancer and CSC DNA instrumental in cancer diagnosis.

FIG. 29C shows MCMM for cancer diagnosis using cancer DNA shows 85% accuracy.

FIG. 29D shows MCMM for cancer diagnosis using CSC DNA shows 100% accuracy, validating the applicability of CSC methylation as a viable marker for cancer diagnosis.

FIG. 30A shows a schematic diagram of how a sensor showed signature profiles of multiple cancer types.

FIG. 30B shows a schematic representation of the detection methodology.

FIG. 30C shows an analysis with pre-clinical cell lines demonstrated clear clustering for multiple cancer types.

FIG. 30D shows tissue of origin diagnosis directly from patient blood plasma with random forest classifier showed 84.4% accuracy.

FIG. 31A shows a schematic Representation of Metastatic Progression.

FIG. 31B shows MCMM of tumor DNA was used as training data.

FIG. 31C shows cancer progression was diagnose with multiple types of cancers (i) Breast Cancer (ii) Lung Cancer (iii) Colorectal Cancer.

FIG. 31D shows random forest classifier demonstrated (i) distinct clustering (ii) 89.5% sensitivity and 100% specificity (iii & iv) ROC with area under the curve 1.00 showing highest accuracy

FIG. 32A shows MCMM of tumor DNA was used as training data.

FIG. 32B shows cancer site of progression was diagnose with multiple types of cancers: (i) Breast Cancer (ii) Lung Cancer (iii) Colorectal Cancer.

FIG. 32C shows a random forest classifier demonstrated (i) distinct clustering.

FIG. 32D shows performance of random forest classifier demonstrating high sensitivity and specificity for detection of cancer site of progression.

FIG. 33A shows that a simple machine learning (Random Forest) was applied with MCMM of patient tumor DNA as training data and that threshold for detection of nodal metastasis was obtained.

FIG. 33B shows diagnosis of nodal metastatic grade across different types of cancer: (i) silhouette plot showing the classification of nodal metastasis grade into separate clusters; (ii) ROC curve for the training and validation dataset showcasing AUC of 1.000 for training and validation dataset; (iii, iv) ROC showing area under the curve (0.96) showing very high accuracy; (v) Confusion matrix showing very high sensitivity 94.1% and specificity 71.4%.

FIG. 34A shows a schematic representation of metastatic cascade.

FIG. 34B shows a schematic representation of detection of clinical metastasis directly from patient blood plasma.

FIG. 34C shows detection of metastasis was achieved with 100% sensitivity and 100% specificity.

FIG. 35A shows i) a schematic demonstrating the adsorption of CV molecule on superlattice sensor, ii) HRSEM image of 3D layered superlattice sensor to facilitate adsorption of analytes, iii) LoD experiment using CV exhibits ultra-sensitivity by detection of up to attomolar concentration.

FIG. 35B shows i) a schematic showing capture of EVs on sensor surface; ii) HRSEM image of an EV trapped on the superlattice sensor surface; and iii) LoD experiment demonstrated ultralow detection sensitivity up to 5 EVs/5 μL.

FIG. 36A shows Raman spectra of GBM EVs acquired at 10 random locations on the superlattice sensor surface

FIG. 36B shows the corresponding 2D area plot showing the intensity distribution of FIG. 27A.

FIG. 36C shows a bar chart showing the RSD values calculated for 6 different peaks corresponding to proteins, lipids, and nucleic acids. RSD values observed were below 20%.

FIG. 37A shows i) a schematic showing the non-cancer and CSC derived EV subpopulations and representative SERS spectra obtained; ii) PCA demonstrates the clustering of the EV populations; iii) Heatmap illustrates the variances in the SERS spectra; iv) Altered expression of biomolecules in non-cancer and GBM CSC EVs.

FIG. 37B shows i) a schematic showing the non-cancer and GBM cancer derived EV subpopulations and representative SERS spectra acquired; ii) a PC plot showing two distinct clusters separating cancer and non-cancer EVs; iii) a heatmap displaying the covariances in whole SERS spectra; iv) a heatmap illustrating the variances in the SERS spectra; and iv) altered expression of biomolecules in non-cancer and GBM EVs.

FIG. 38A shows a schematic representation of EVs captured on superlattice surface.

FIG. 38B shows the acquired representative SERS spectra.

FIG. 38C shows a heatmap showing spectral bands of highest variance.

FIG. 38D shows a differential expression of biomolecules in GBM and GBM CSC EVs.

FIG. 38E shows i) PCA analysis showing clustering of EV populations in PC plot; ii) corresponding correlation plot; and iii) PC loading spectra.

FIG. 39A shows a schematic showing clusters of tumor cells and EVs isolated from serum of corresponding patient adsorbed on superlattice surface.

FIG. 39B shows a representative SERS spectra of tumor cells and EVs isolated from serum.

FIG. 39C shows pair plots indicating the similarity in spectral data.

FIG. 39D shows a correlation plot exhibiting minimum variance between SERS data of tumor and EVs.

FIG. 39E shows a heatmap illustrating the spectral bands of high similarity.

FIG. 39F shows a PC plot exhibiting a single cluster in 95% confidence interval.

FIG. 40A shows i) representative spectra of EVs derived from non-cancer cells and GBM CSCs; ii) Hierarchical clustering showing similarities within data; iii) Prediction probabilities for the identification of GBM; iv) Sensitivity (100%) and specificity (100%) curves for a prediction threshold probability of 0.5; v) and vi) ROC curves for prediction of healthy and GBM patient population with an AUC of 1.0.

FIG. 40B shows i) representative SERS spectra of EVs derived from non-cancer cells and GBM cancer cells; ii) Hierarchical clustering showing similarities within SERS data of non-cancer and cancer EVs; iii) Prediction probabilities for the identification of GBM; iv) Sensitivity (90%) and specificity (100%) curves for a prediction threshold probability of 0.5; v) and vi) ROC curves for prediction of healthy and GBM patient population with an AUC>0.9.

FIG. 41 shows a schematic representation of holistic analysis of GSC-DNA for GBM diagnosis.

FIG. 42A shows a bulk tumour consisting of heterogenous populations from which DNA is isolated and data is gathered.

FIG. 42B shows a comparison of the SERS spectra of the DNA from glioblastoma cancer cell and CSC on the meta sensors.

FIG. 42C shows box plots showing comparison of normalized peak intensities of C+T, A, G and A+G in cancer and CSC cell-derived DNA.

FIG. 42D shows a heatmap showing the clear distinction between the cancer DNA and CSC DNA.

FIG. 42E shows PCA scatter plot showing the differences and their clustering.

FIG. 43A shows SERS spectra of the GSC DNA at different concentrations serially diluted from 10%.

FIG. 43B shows regression analysis of the peak intensities of the A, G, C+T.

FIG. 43C shows GSC DNA concentration of the patient samples.

FIG. 43D shows predicted vs actual concentration of the GSC DNA.

FIG. 43E shows a Pythagorean tree showing the clustering of the samples based on GSC DNA levels.

FIG. 44A shows a heat map showing the correlations between the GBM DNA and GSC DNA with Tumor derived DNA

FIG. 44B shows a scatter plot showing the correlations between the GBM DNA and GSC DNA with Tumor derived DNA.

FIG. 44C shows a heatmap showing the similarities between the tumor DNA, patient serum samples & cfDNA.

FIG. 44D shows a scatter plot showing the similarities between the tumor DNA, patient serum samples & cfDNA.

FIG. 44E shows similarities between in-vitro healthy cell genomic DNA and cfDNA (f) similarities between healthy serum, cfDNA and in-vitro healthy cfDNA.

FIG. 45A shows a scheme showing the overview of liquid biopsy detection.

FIG. 45B shows a scatter plot showing the differences between healthy and cancer patients detected without including GSC DNA in training data for the random forest analysis.

FIG. 45C shows a sensitivity and specificity curve for the random forest classification.

FIG. 45D shows a scatter plot showing the differences between healthy and cancer patients detected by including GSC DNA in training data for the random forest analysis.

FIG. 45E shows a sensitivity and specificity curve for the random forest classification.

FIG. 46 shows i) a schematic showing the MLP-LIBT model architecture; ii) and iii) accuracy and loss curves generated during training and validation steps respectively; and iv) Probabilities with which the testing data were classified for 9 different locations

FIG. 47A shows a dendrogram clustering Raman data based on the tumor location trait to detect the outliers.

FIG. 47B shows a cluster dendrogram generated to identify modules of highly correlated Raman peaks.

FIG. 47C shows a scale free topology model—determination of soft-threshold power based on the fit.

FIG. 48A shows a correlation plot showing the module-trait relationship—Blue module shows highest positive correlation for location trait.

FIG. 48B shows a scatter plot showing Raman significance of blue module (p=1.8×10¹⁷) for location trait.

FIG. 48C shows a network TOM heatmap plot for selected Raman peaks. The plot shows the degree of correlation within each module.

FIG. 49A shows a network of Raman peaks showing highest correlation with location trait. All peaks in the network expressed a weight above 0.6.

FIG. 49B shows as bar chart depicting Raman peak assignment corresponding to most significant peaks based on their weights.

Further aspects and features of the example embodiments described herein will appear from the following description taken together with the accompanying drawings.

### DESCRIPTION OF EXAMPLE EMBODIMENTS

Various apparatuses, methods and compositions are described below to provide an example of at least one embodiment of the claimed subject matter. No embodiment described below limits any claimed subject matter and any claimed subject matter may cover apparatuses and methods that differ from those described below. The claimed subject matter are not limited to apparatuses, methods and compositions having all of the features of any one apparatus, method or composition described below or to features common to multiple or all of the apparatuses, methods or compositions described below. It is possible that an apparatus, method or composition described below is not an embodiment of any claimed subject matter. Any subject matter that is disclosed in an apparatus, method or composition described herein that is not claimed in this document may be the subject matter of another protective instrument, for example, a continuing patent application, and the applicant(s), inventor(s) and/or owner(s) do not intend to abandon, disclaim, or dedicate to the public any such invention by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practiced without these specific details. In other instances, well-known methods, procedures, and components have not been described in detail so as not to obscure the example embodiments described herein. Also, the description is not to be considered as limiting the scope of the example embodiments described herein.

It should be noted that terms of degree such as “substantially”, “about” and “approximately” as used herein mean a reasonable amount of deviation of the modified term such that the end result is not significantly changed. These terms of degree should be construed as including a deviation of the modified term, such as 1%, 2%, 5%, or 10%, for example, if this deviation does not negate the meaning of the term it modifies.

Furthermore, the recitation of any numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation up to a certain amount of the number to which reference is being made, such as 1%, 2%, 5%, or 10%, for example, if the end result is not significantly changed.

It should also be noted that, as used herein, the wording “and/or” is intended to represent an inclusive-or. That is, “X and/or Y” is intended to mean X, Y or X and Y, for example. As a further example, “X, Y, and/or Z” is intended to mean X or Y or Z or any combination thereof. Also, the expression of A, B and C means various combinations including A; B; C; A and B; A and C; B and C; or A, B and C.

The example embodiments of the devices, systems or methods described in accordance with the teachings herein may be implemented as a combination of hardware and software. For example, the embodiments described herein may be implemented, at least in part, by using one or more computer programs, executing on one or more programmable devices comprising at least one processing element and at least one storage element (i.e. at least one volatile memory element and at least one non-volatile memory element). The hardware may comprise input devices including at least one of a touch screen, a keyboard, a mouse, buttons, keys, sliders and the like, as well as one or more of a display, a speaker, a printer, and the like depending on the implementation of the hardware.

It should also be noted that there may be some elements that are used to implement at least part of the embodiments described herein that may be implemented via software that is written in a high-level procedural language such as object oriented programming. The program code may be written in MATLAB, Julia, Python, C, C⁺⁺ or any other suitable programming language and may comprise modules or classes, as is known to those skilled in object oriented programming. Alternatively, or in addition thereto, some of these elements implemented via software may be written in assembly language, machine language or firmware as needed. In either case, the language may be a compiled or interpreted language.

At least some of these software programs may be stored on a computer readable medium such as, but not limited to, a ROM, a magnetic disk, an optical disc, a USB key and the like that is readable by a device having a processor, an operating system and the associated hardware and software that is necessary to implement the functionality of at least one of the embodiments described herein. The software program code, when read by the device, configures the device to operate in a new, specific and predefined manner in order to perform at least one of the methods described herein.

Furthermore, at least some of the programs associated with the devices, systems and methods of the embodiments described herein may be capable of being distributed in a computer program product comprising a computer readable medium that bears computer usable instructions, such as program code, for one or more processing units. The medium may be provided in various forms, including non-transitory forms such as, but not limited to, one or more diskettes, compact disks, tapes, chips, and magnetic and electronic storage. In alternative embodiments, the medium may be transitory in nature such as, but not limited to, wire-line transmissions, satellite transmissions, internet transmissions (e.g. downloads), media, digital and analog signals, and the like. The computer useable instructions may also be in various formats, including compiled and non-compiled code.

The following description is not intended to limit or define any claimed or as yet unclaimed subject matter. Subject matter that may be claimed may reside in any combination or sub-combination of the elements or process steps disclosed in any part of this document including its claims and figures. Accordingly, it will be appreciated by a person skilled in the art that an apparatus, system or method disclosed in accordance with the teachings herein may embody any one or more of the features contained herein and that the features may be used in any particular combination or sub-combination that is physically feasible and realizable for its intended purpose.

Recently, there has been a growing interest in developing new technologies of early cancer detection and prognosis. Specifically, there has been a growing interest in identifying new biomarkers in physiological (e.g. blood) samples from humans for early cancer detection and prognosis.

Currently, there are at least two types of biomarkers that can be detected from blood samples. These biomarkers are generally referred to as cell-component biomarkers and immune-cell biomarkers.

Cell-component biomarkers currently include, but are not limited to, extracellular vesicles of circulating cancer initiating cells. Currently, extracellular vesicles may be used to diagnose a type of a cancer, its primary location, its stage, and/or whether the cancer is metastatic. Additionally, extracellular vesicles may be used to provide real-time (e.g., same day test results) monitoring of treatment effect and prognosis for asymptotic individuals.

Cell-component biomarkers also currently include, but are not limited to, cell-free DNA. Cell-free DNA may also be used to diagnose a type of a cancer, its primary location, its stage, and/or whether the cancer is metastatic. Additionally, cell-component biomarkers may be used to diagnose tumor burden, perform therapy monitoring, determine prognosis after therapy and to provide a therapy efficacy assessment.

Cell-component biomarkers also currently include, but are not limited to, methylated cell-free DNA. Methylated cell-free DNA may be used to diagnose the origin of a metastatic tumour, the metastatic potential of the primary tumour and the possible type of metastatic site.

Cell-component biomarkers also currently include, but are not limited to, methylated tumour DNA, which may be used to diagnose the aggressiveness of the tumour, the type of tumour, the metastatic potential and the composition of the tumour such as, for example, where there is a presence of cancer stem cells (CSCs) and metastatic CSCs in the tumour microenvironment.

Immune-cells biomarkers include but are not limited to t-cells and other types of cells from the immune systems, such as but not limited to Natural Killer (NK) cells, myeloid derived suppressor cells (MDSCs) and tumor associated macrophages.

Generally, there are three major types of T cells: CD 3+ (total T cells), CD 4+ (i.e. naive T cells/unactivated) and CD 8+ (i.e. cytotoxic T cells). The presence of high-density CD 8+ T cells are correlated with cancer stage and the ability of the patient to respond to immunotherapy. Increases in the number of CD 3+ and CD 4+ T cells generally results in a reduction of monocytes, which thereby promotes tumor growth, migration and invasion.

Currently, in the prior art, no definitive biomarker exists to predict the outcome of immune system activity/immune infiltration potential of cancer during clinical decision making.

However, in accordance with the teachings herein, methods of establishing cell-component biomarkers using standard templates are described. To establish the cell-component biomarkers, the cell-component biomarkers are first isolated (e.g. from patient tissue biopsy or a cancer cell line). Following isolation, Raman spectral profiles are collected to build a standard template.

Establishing immune-cell standard templates (e.g. templates for immunoprofiling) are described herein as follows: T cells isolated from a buffy coat from normal blood are used to establish the surface-enhanced Raman scattering (SERS) characteristics of the T cells. For example, CD 4+ T cells and CD8+ T cells are distinctly different in size, have different intracellular calcium dynamics and different cytokine secretion, thereby providing the potential to differentiate using their SERS signatures.

Templates of cells from the immune system such as, but not limited to, NK cells, myeloid derived suppressor cells (MDSCs) and tumor associated macrophages can be establish in the same manner. Here, the presence and concentration of each of these immune cell types in the blood can be correlated with the cancer stage, metastatic potential, therapy efficacy and the development of adoptive immunity to cancer.

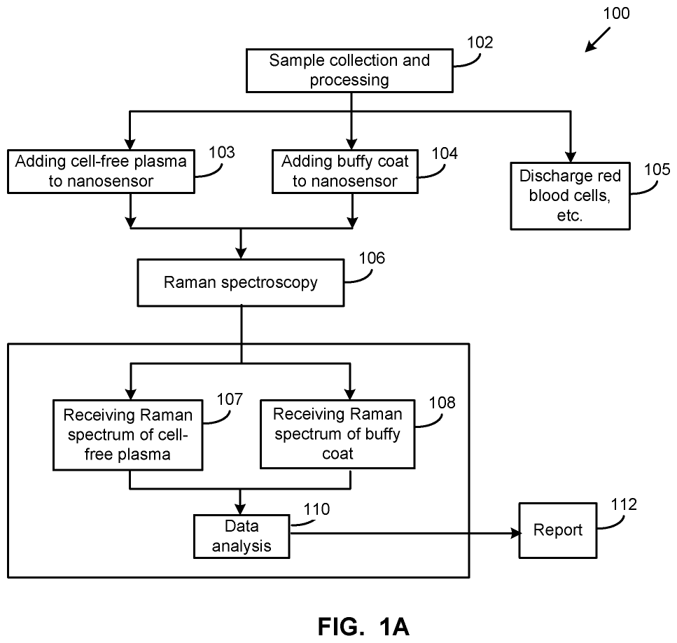

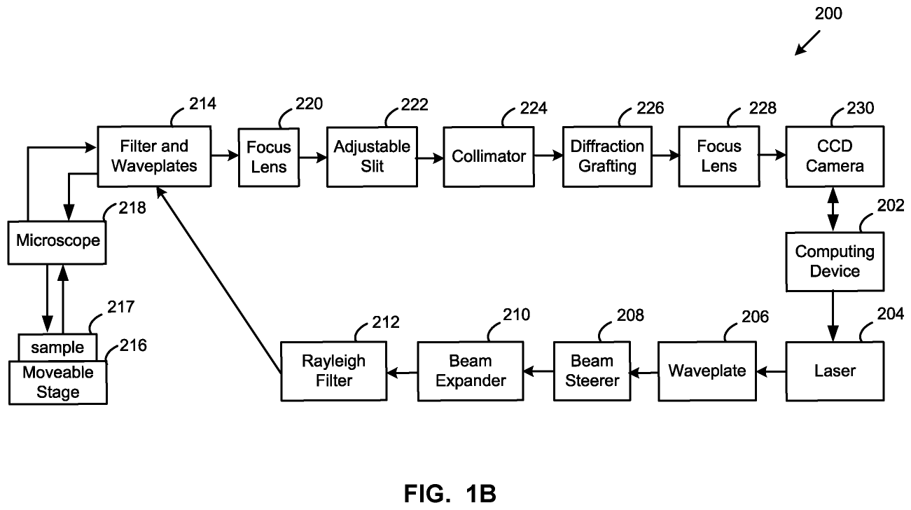

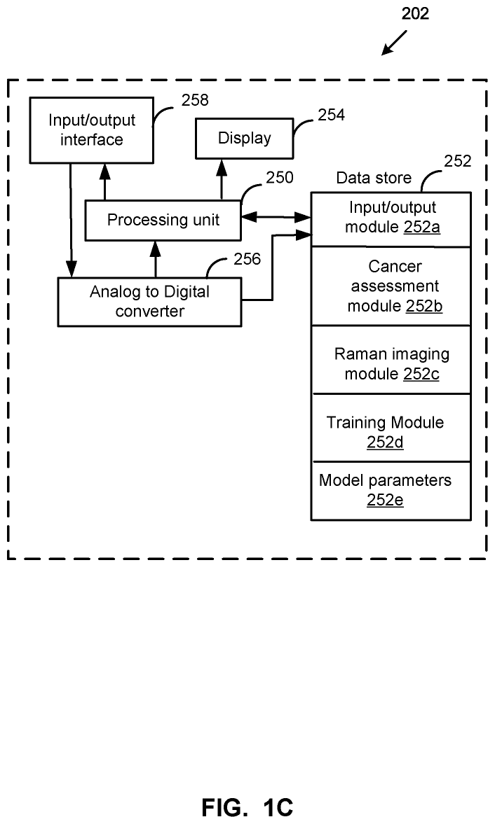

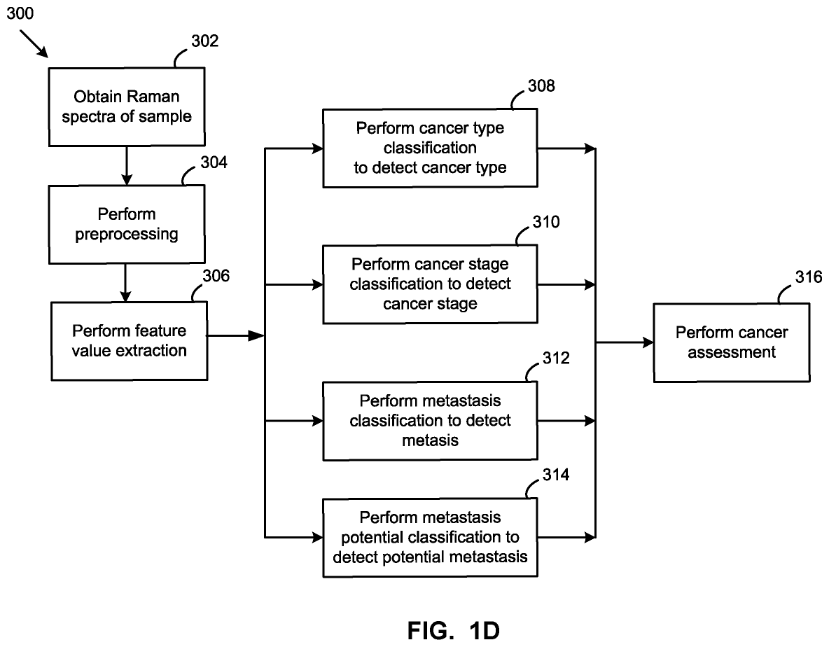

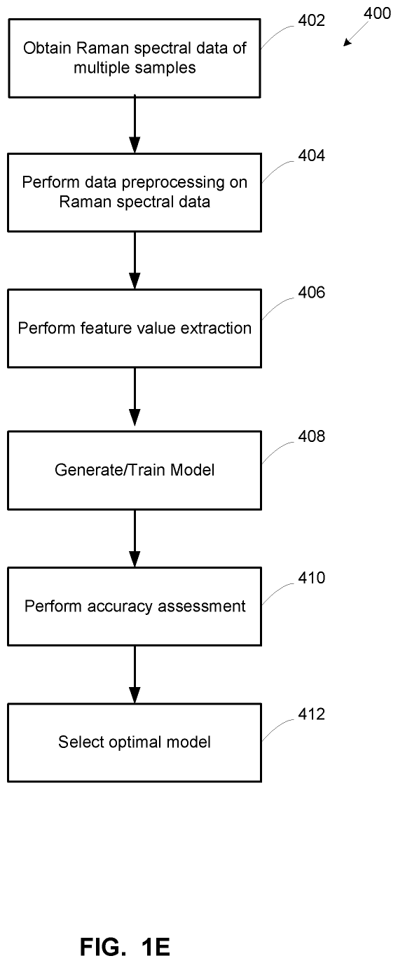

Referring now to the figures, FIG. 1A shows an example embodiment of a method 100 of diagnosing cancer in a patient in accordance with the teachings herein. FIG. 1B shows an example embodiment of a system 200 for obtaining Raman spectra of biomarkers captured by nanosensors, as described in the methods described herein.

In FIG. 1A, at step 102, a fluid sample is collected (e.g. raw blood plasma; plasma that has been collected from a patient) and processed to produce a volume of cell-free plasma, a volume of buffy coat and a volume of remaining products (e.g. red blood cells, etc.). The processing of step 102 may be done using suitable techniques such as, but not limited to, density gradient centrifugation, for example, to obtain the cell-free blood plasma, buffy coat and remaining products (e.g. red blood cells, etc.)

In at least one embodiment, at step 103, a volume of the blood plasma (e.g. cell-free blood plasma) produced from step 102 is dropped onto a first nanosensor for detection of cell-component biomarkers. In at least one embodiment, the volume of the blood plasma comprises cell component biomarkers such as extracellular vesicles of circulating cancer stem cells, cell-free DNA, methylated cell-free DNA, methylated tumor DNA, exosomes, DNA of cancer stem cells or combinations thereof.

In at least one embodiment, the volume of blood plasma added to the nanosensor is in a range of about 10 microliters (μL).

The nanosensors used herein for surface enhanced Raman scattering for biomolecule detection include one or more nanoparticles (e.g. nanoprobes). The nanoparticles are made of materials such as but not limited to gold, silver, platinum, titanium, silicon, aluminum, nickel, and/or graphite. The nanoparticles described herein may also be referred to as quantum dots (i.e. nanoparticles with particle size less than about 5 nm).

Unlike all other types quantum dots, the quantum dots of nanosensors described herein are generally non-toxic, making them particularly suitable for biomedical applications. In addition, the dots are free of contaminations and generally do not react/interfere with target molecules.

The quantum dots of the nanosensors described herein are smaller and have a unique structure (e.g. have a high vacancy density of crystalline nanoparticles) compared to conventional quantum dots, which translates to higher detection sensitivity and pushes the limit of detection to a lower concentration. Therefore, previously undetectable traces (undetectable because of their low concentration) may be detected using quantum dots of the nanosensors described herein.

In at least one embodiment, the nanosensor may amplify extremely weak signals of biomarkers such as but not limited to extracellular vesicles of circulating stem cells (CSCs) and/or the other biomarkers noted herein. The nanosensor includes one or more self-assembled, three dimensional nanoprobes that can detect a biomolecular configuration of extremely low levels of extracellular vesicles associated with CSCs (e.g. 10 extracellular vesicles in 10 μl of solution), for example. In at least one embodiment, the nanosensors (i.e. nanoprobes) promote trapping of extracellular vesicles, which results in an overall increase in surface area of the extracellular vesicles, and permit drainage of fluids improving extracellular vesicles-surface interaction. The nanosensor is a three-dimensional and porous network of nanoprobes. The interconnected crisscross of nanosensors can act as a trapping and screening device. In addition to biocomponents getting trapped by the nanosensor, fluids are easily drained from the nanosensor, thereby improving the interaction of the biocomponents with the nanosensors.

Generally, the nanosensors used in the methods and systems described herein have a three-dimensional (3D) structure comprising self-assembled closed rings and bridges which causes nanoparticles to aggregate together. Between the self-assembled closed rings and bridges are pores that are interconnected. The ring size is roughly the wavelength of a laser beam used to create the sensor.

In at least one embodiment, the nanoparticles contain rich crystalline defects. In at least one embodiment, the nanoparticle size is tuneable from about 100 nm to about 1 nm. For example, in at least one embodiment, the nanoparticle size may be less than about 1 nm.

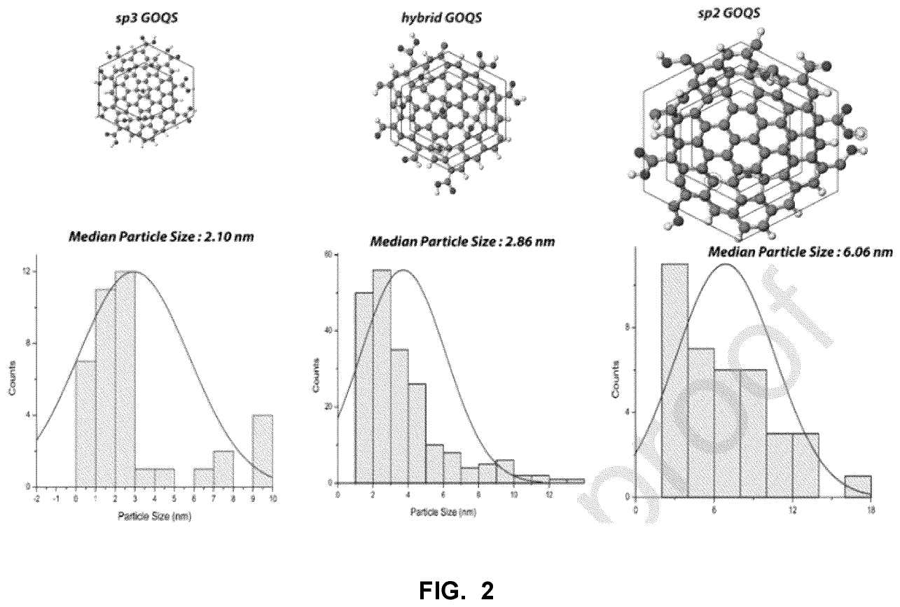

FIG. 2 shows some example embodiments of carbon particles (i.e. nanosensors) used on nanosensors in the systems and methods described herein, and their median particle size distributions. Generally, the nanoparticles have a size range of about 2 nm to about 6 nm. Specifically, the nanoparticles provided in FIG. 2 are sp³ graphene oxide quantum dots (GOQS) having a median particle size of 2.10 nm, hybrid GOQS having a median particle size of 2.86 nm and sp² GOQS having a median particle size of 6.06 nm.

In at least one embodiment, the nanosensors used in the methods and systems described herein are fabricated by the methods described in U.S. Provisional Patent Application No. 63/059,079 filed 30 Jul. 2020 entitled “ULTRASHORT LASER SYNTHESIS OF NANOPARTICLES OF ISOTOPES”, the contents of which are incorporated herein by reference.

In one embodiment, at step 104, optionally, a volume of the buffy coat produced from step 102 may be dropped onto a second nanosensor to detect immune-cell biomarkers. In at least one embodiment, the volume of buffy coat comprises immune cell biomarkers such as circulating tumor cells, circulating stem cells, and/or immune cells such as but not limited to T cells (e.g. CD 3+, CD 4+ and/or CD 8+), natural killer (NK) cells, β cells, myeloid derived suppressor cells or combinations thereof.

In at least one embodiment, the volume of the buffy coat added to the nanosensor is in a range of about 10 uL.

In accordance with the teachings herein, the nanosensors that are used allow for the detection of immune cell biomarkers and/or cell component biomarkers in low concentrations that were previously not detectable. This is advantageous as the detection of one or more cell-component biomarkers can be used to diagnose one or more cancer characteristics such as the type of a cancer, the cancer primary location, the cancer stage, whether the cancer is metastatic, or a combination of these features. On the other hand, the detection one or more of immune cell biomarkers can be used to determine the cancer stage and/or the ability of a patient to respond to immunotherapy.

Optionally, at a step 105, the remaining products produced during step 102 may be discarded.

At step 106, in at least one embodiment, after a first incubation time, the first nanosensor including the volume of blood plasma is scanned under a Raman microscope to obtain a Raman spectrum.

In at least one embodiment, the first incubation time is in a range of about 1 to about 2 minutes. The length of the first incubation time is based on the time needed for the biomarkers to adsorb onto the surface of the nanosensors. The first incubation time may be determined empirically.

Also at step 106, in at least one embodiment, after a second incubation time, the second nanosensor including the volume of buffy coat is scanned under a Raman microscope to obtain a Raman spectrum.

In at least one embodiment, the second incubation time is in a range of about 1 to about 2 minutes. Again, the length of the second incubation time is based on the time needed for the biomarkers to adsorb onto the surface of the nanosensors. The second incubation time may be determined empirically.

At step 107 one or more Raman spectra of the volume of cell-free plasma on the nanosensor is generated by a Raman spectroscopy system.

At step 108 one or more Raman spectrum of the volume of buffy coat on the nanosensor is generated by a Raman spectroscopy system.

One example of a Raman spectroscopy system for obtaining Raman spectra of the samples on a nanosensor of steps 107 and/or 108 of method 100 is provided in FIG. 1B. FIG. 1B shows on example embodiment of a system 200 having one or more hardware components used in obtaining Raman spectra of biomarkers on nanosensors during, for example, step 106 of the method of FIG. 1A, according to at least one example embodiment described herein.

The system 200 includes a computing device 202, an excitation pathway including a laser 204, a waveplate 206, a beam steerer 208, a beam expander 210, a Rayleigh filter 212, filter and waveplates 214, and a moveable stage 216. The nanosensor is on the moveable stage 216. The system 200 further includes a return pathway including a microscope 218, the filter and waveplates 214, a focus lens 220, an adjustable slit 222, a collimator 224, a diffraction grating 226, a focus lens 228 and a CCD camera 230. In at least one embodiment, the system 200 is a confocal Raman microscope system with, for example, an excitation wavelength of 785 nm. This microscope system provides all of the components shown in FIG. 1B except for the computing device 202.

During use, the computing device 202 includes one or more software programs (see e.g. FIG. 1C) for performing Rayleigh imaging and analysis of a sample 217, such as the plasma or buffy coat sample, that has been placed on the nanosensor. Specifically, the computing device 202 is coupled to the laser 204 and the CCD camera 230 through 1/O hardware including possibly an Analog to Digital Converter and/or USB cables, so that the computing device 202 may control the generation of laser pulses by the laser 204 and the recordal of images by the CCD camera 230. Laser pulses from the laser 204 are transitory optical beams of light that are polarized by the waveplate 206 and then steered by the beam steerer 208. The steered optical beams are then enlarged in diameter by the beam expander 210 and then filtered by the Rayleigh filter 212 and the filter and waveplates 214 to purify scattered light by which is then provided to the microscope 218, which may be a standard microscope, that includes focusing lens for focusing the excitation light pulses onto the sample 217.

The excitation light pulses excite molecules in the sample 217 and the excited molecules which emit scattered photons that are at different energies and different frequencies. There is also a change in the electric dipole-electric dipole polarization and a resulting Raman scattering which is proportional to the polarization change. The scattered photons, referred to as a Raman signal, are then focused and purified by the optical elements in the return pathway prior before reaching the CCD camera 230. The CCD camera 206 then records the images and transmits the images to the computing device 202 so that it can perform processing, as described below.

For example, referring now to FIG. 1C, shown therein is an example embodiment of a block diagram of an example embodiment of a computing device 202 and software that is used with the hardware setup of FIG. 1B. It should be noted that this is one example embodiment and in other embodiments the computing device 202 may have more or less components or alternative layouts in other embodiments. The computing device may be a desktop computer or other suitable computing device which is able to communicate with some of the hardware components in FIG. 1B perhaps wirelessly in which case the computing device may be a tablet.

The computing device 202 generally comprises a processing unit 250, an Analog to Digital Converter (ADC) 256, a data store 252, a display 254 and an input/output interface 258 which may be coupled to various peripheral components such as the laser 204 and the CCD camera 230, or a prepackaged Raman microscope which includes these hardware components. The computing device 202 may also include a power unit (not shown) or be connected to a power source to receive power needed to operate its components.

The processing unit 250 is operatively coupled to the other components of the computing device 202 for controlling various operations and performing certain functions, such as setting or modifying stimulus parameters for the laser 204 (i.e. wavelength and intensity), the data acquisition process (i.e. controlling image acquisition, etc.) and assessing patient samples for various aspects of cancer as described herein.

The processing unit 250 can be any suitable processor, controller or digital signal processor that can provide sufficient processing power depending on the operational requirements of the computing device 202 as is known by those skilled in the art. For example, the processing unit 250 may be a high performance processor. In alternative embodiments, the processing unit 250 may include more than one processor with each processor being configured to perform different dedicated tasks.

The data store 252 includes volatile and non-volatile memory elements such as, but not limited to, one or more of RAM, ROM, one or more hard drives, one or more flash drives or some other suitable data storage elements. The data store 252 may be used to store an operating system and programs as is commonly known by those skilled in the art. For instance, the operating system provides various basic operational processes for the processing unit 252 and the programs include various operational and user programs so that a user can interact with the computing device 202 to perform Raman imaging of a sample and subsequent cancer assessment of the sample, to determine and/or update models (e.g. through training) used in the cancer assessment or a combination of both of these operations.

The data store 252 may also include software code for implementing various components for Raman imaging, model training and various aspects of cancer assessment in accordance with the teachings herein as well as storing values for various operational parameters that are used for Raman imaging. For example, the data store 252 can include programs for implementing an input/output module 252a, a cancer assessment module 252b, a Raman imaging module 252c, and a training module 252d. It should be noted that there may be other embodiments in which the software modules may be organized differently; however, the same functions as described herein are performed.

The input/output module 252a can include program instructions for receiving acquired Raman image data and user control data. The input/output module 252a can also include program instructions for outputting and/or storing raw Raman image data, preprocessed Raman image data, and cancer assessment data.

The cancer assessment module 252b includes program instructions for obtaining and preprocessing Raman spectral data for a patient sample, performing feature extraction on the preprocessed Raman spectral data and providing feature extraction values to various models including classifiers for detecting various cancer characteristics that can be used for providing a cancer assessment for the patient sample based on one of more of the biomarkers described herein. The cancer characteristics may include cancer type, cancer stage, cancer metastasis, potential cancer metastasis or any combination thereof. If the analysis is done based on two or more biomarkers then the results from each biomarker can be combined to provide a cancer assessment with improved accuracy compared to basing the analysis on only one biomarker. The operation of the cancer assessment module 252b is further described with reference to FIG. 1D.

The Raman imaging module 252b is used to control the generation of excitation laser pulses by the laser 204 and recordal of resulting images when the patient sample 217 is on the moveable stage 216. The Raman images may then be directly processed by the cancer assessment module 252b and optionally stored on the data store 252.

The training module 252d includes program instructions using training data to obtain determine the models (e.g. classifiers) used by the cancer assessment module 252b as well as update these models over time as more training data is acquired. The training data may be obtained using the hardware setup of FIG. 1B or it may be obtained using other means and then accessed by the input/output module 252a whenever training is performed. In some embodiments, the training data may be stored in the data store 252. Once the training is performed for the models, the model parameters 252e may be stored in the data store 252. The operation of the training module 252d is described in more detail with respect to FIG. 1E.

The display 254 can be any suitable device for displaying images and various types of information such as an LCD monitor or touchscreen display. The input/output interface 258 can be various ports such as one of more of USB, Firewire, serial and parallel ports, for example, that may be coupled with various peripheral devices used for the input or output of data. Examples of these devices include the laser 204 and CCD camera 230 and may also include, but are not limited to, a keyboard, a mouse, a trackpad, a touch interface, and a printer or any combination thereof. The Analog to Digital Converter 256 may be needed to convert any analog data that is received by the input/output interface 258 into digital data.

Returning back to FIG. 1A, at step 110, the computing device 202 (i.e. a processor thereof) performs processing to analyse the one or more Raman spectra obtained from steps 107, 108. For instance, in one embodiment, at step 110, the one or more obtained Raman spectrum is/are compared to one or more template Raman spectra using correlation analysis, when the processor is executing one of the computer programs.

In at least one embodiment, the correlation analysis of the one or more obtained Raman spectra with the template Raman spectrum includes a comparison of signature peaks of Raman bands of the obtained Raman spectrum with signature peaks of Raman bands of the template Raman spectrum. The template Raman spectrum includes an average spectrum of a plurality of individual Raman spectra from samples of known cancer characteristics.

Raman spectra of plasma/cfDNA/exosomes, for example, contains rich information. This information is typically in the form of signature peaks. For example, the peaks at a particular wavenumber typically represent a type of biomolecule. Some representative Raman assignments are provided here as an example. For instance, a peak at 782 cm⁻¹ is assigned to the ring breathing mode of DNA/RNA bases, a peak at 1445 cm⁻¹ CH₂ is assigned to bending modes of proteins, a peak at 1670 cm⁻¹ is assigned to stretching modes of C═C in lipids, and a peak at 2850 cm-1 is assigned to CH₂ symmetric stretch in lipids. The Raman assignments are well established by known literature. It should be understood that specific signature peaks of the spectra can be selected depending on the application of the data.

In at least one embodiment, the correlation analysis may include use of one or more correlation analysis tools. Some of the tools for performing correlation analysis may include: Pearson's correlation analysis, Spearman correlation test, Heat maps and/or plotting correlation matrix of eigenvalues using artificial neural network. In at least one embodiment, during the correlation analysis, ten measurements from one sample are obtained.

In at least one embodiment, a large number of peaks in any given spectrum may be selected. The number of peaks may be determined by the molecular vibrations in each biomarker. Generally, a minimum of three peaks are considered for every biomarker used. In at least one embodiment, the number of peaks used may be in a range of 3 to 10. In at least one embodiment, there may be overlap of many peaks in the Raman spectra of relatively large macromolecules like proteins, lipids and/or nucleic acids. Thus, many inaccuracies may be introduced into the analysis when the spectra are analyzed qualitatively and certain peaks are assigned to specific biochemical components. These errors are introduced from visual inspection and guessing to determine biochemical components from changes in intensity as there always is a possibility of combinational contribution from several components contributing to one peak. Also, there is a possibility of loss of important information from omitted regions of spectra. To overcome these errors, chemometric methods of multivariate data analysis can be employed, as is described with reference to FIGS. 1C and 1D.

In at least one embodiment, a diagnosis may be made if there is high similarity between the sample Raman spectrum and the template Raman spectrum. For instance, in at least one embodiment, if the correlation analysis of the obtained Raman spectrum (from the patient sample) with the template Raman spectrum demonstrates correlation of 65% or above, a diagnosis may be made based on the underlying cancer characteristics of the template Raman spectrum.

Establishing immune-cell standard templates (e.g. templates for immunoprofiling) are described herein as follows: T cells isolated from a buffy coat from normal blood are used to establish the surface-enhanced Raman scattering (SERS) characteristics of the T cells. For example, CD 4+ T cells and CD8+ T cells are distinctly different in size, have different intracellular calcium dynamics and different cytokine secretion, thereby providing the potential to differentiate using their SERS signatures.

Templates of cells from the immune system such as, but not limited to, NK cells, myeloid derived suppressor cells (MDSCs) and tumor associated macrophages can be established in the same manner. Here, the presence and concentration of each of these immune cell types in the blood can be correlated with the cancer stage, metastatic potential, therapy efficacy and the development of adoptive immunity to cancer.

In at least one embodiment, the obtained Raman spectrum/spectra may be compared to more than one template simultaneously to make a multiplex diagnosis. In at least one embodiment, the comparison of the obtained Raman spectrum/spectra to the template(s) may be completed by a computer program. For instance, in at least one embodiment, Python™ and/or Matlab® modules may be used for data comparison and data interpretation.

Referring now to FIG. 1D, shown therein is a flow chart of a method 300 for performing a cancer assessment on a patient sample using Raman spectra obtained therefrom that may be performed by software of the computing device 202 according to at least one example embodiment described herein. In alternative embodiments some aspects of the method 300 may be modified. For example, while the application of four classifiers is shown in FIG. 1D, it is possible that some of the classifiers are not applied on the basis of the underlying biomarkers that are used for performing classification.

At acts 302 and 304, the Raman spectrum of the patient sample are obtained and preprocessed. This may be done using the hardware setup of FIG. 1B or this Raman spectrum may have already been obtained in which case it is accessed from storage and might already have been preprocessed. The preprocessing involves removing any amplitude shifts that are due to the baseline in the Raman spectrum data when this data is first acquired. The baseline may induce uneven amplitude shifts across different wavenumbers and negatively affect the analysis that is done by the cancer assessment module 252b. The baseline correction that is performed during preprocessing at act 304 is known to those skilled in the art.

At act 306, feature extraction is performed on the preprocessed Raman spectrum to obtain values for features that are used by the models in later steps of method 300. The feature extraction results in a reduction in the dimensionality of the data. The feature extraction may be performed using several different techniques. For instance, at step 306, Principal Component Analysis (PCA) may be used as a non-parametric approach that does not need any explicit background model. In principal component analysis, the input Raman spectral data is decomposed and the output that is generated is the principal components. The first principal component can be defined as a direction maximizing the variance. The i^{th} principal component is orthogonal to the first principal component maximizing the variance. In short, principal components are the eigenvectors of the covariance matrix of the input Raman spectral data. One can determine how many principal components are to be used for analysis ignoring the rest of the components. Known techniques may be used to perform the PCA to produce a certain number of principal components such as 10 principal components, for example. In at least one embodiment, described herein, the components that show maximum amount of data are used (e.g. usually three to five components are selected, which represent at least 70% of spectral data). The principal components are the features that are used as inputs to the classifiers. The principal components that are calculated can be further used for solving the problem of classification of one or more components (e.g. extracellular vesicles) that are detected after the sample is on the nanosensor and the Raman image is captured.

Alternatively, at act 306, Multivariate Curve Resolution analysis (MCR) may be used to provide an estimation of contributions of pure ingredients with mixed measurements (for example, a mixture of various types of cfDNA in a plasma sample) providing information on component profiles to generate scientifically meaningful information, which in this case are the features that are then provided to the classifiers. In this way, interpretation of results from complex and large data gathered from Raman spectra may be easily understood.

MCR analysis generally provides more accurate information on the pure components in a mixture compared to the PC analysis. Therefore, by applying it to the extracellular vesicles, one can derive the variation in the pure components of extracellular vesicles. Alternatively, PC analysis is faster than MCR analysis. However, it should be understood that the choice of whether to perform PC analysis or MCR analysis depends on which type of analysis was used in generating/training the classifiers (e.g. classification models). For example, if PC analysis was used to generate/train the cancer type classifier used in act 308 then PC analysis is performed at act 306 and the extracted feature values are input to act 308 meanwhile if MCR analysis was used to generate/train the cancer stage classifier used in act 310 then MCR analysis is performed at act 306 and the extracted feature values are input to act 310. Accordingly, it is possible that both PC and MCR analysis may be performed in act 306 if PC and MCR analysis were each used in determining/training at least one of the classifiers used in acts 308 to 314.

At act 308, the extracted feature values from act 306 are provided as input to a cancer type classifier which provides probabilities for different cancer type classes that are used to detect whether at least one type of cancer is present in the patient sample. This detection result is then provided to act 316 where it is incorporated into the cancer assessment that is performed. It should be noted that if no cancer type is detected at act 310, then there is no need to perform acts 310 to 314.

At act 310, the extracted feature values from act 306 are provided as input to a cancer stage classifier which provides probabilities for different cancer stage classes that are used to detect what the cancer stage is if there is cancer present in the patient sample. This detection result is then provided to act 316 where it is incorporated into the cancer assessment that is performed.

At act 312, the extracted feature values from act 306 are provided as input to a metastasis classifier which provides probabilities for whether the cancer is metastasized or not when there is cancer present in the patient sample. This detection result is then provided to act 316 where it is incorporated into the cancer assessment that is performed.

At act 314, the extracted feature values from act 306 are provided as input to a potential metastasis classifier which provides probabilities for potential cancer metastasis when there is cancer present in the patient sample. This detection result is then provided to act 316 where it is incorporated into the cancer assessment that is performed.

It should be noted that one or more of acts 308 to 314 may be optional in some embodiments.

At act 316, the detection results from acts 308 to 314 which were performed (as some might be optional) are used to provide a cancer assessment, which might be considered to be a cancer diagnosis for the patient based on the analysis of the patient sample. For instance, the cancer assessment, might be that the patient has a certain cancer type, a certain cancer stage, whether the cancer has metastasized and the potential for cancer metastasis to occur. For example, the patient may have breast cancer that is at stage 4 which has metastasized and based on further potential metastasis, the patient may be given a probably of survival of 5 years.

The classification models that are used in acts 308 to 314 can be based on a machine learning model. For example, the machine learning model may be based on Partial Least Square Discriminant Analysis (PLSDA), Support Vector Machine Discriminant Analysis (SVMDA), or an PLSDA or an Artificial Neural Network topology. Alternatively, other types of machine learning techniques might be used such as, but not limited to, Convolutional Neural Networks, and the Random Forrest method, for example.

In an alternative embodiment, the classification models may be based on a deep learning model in which case the feature extraction step of act 306 does not need to be performed since the deep learning model can perform feature extraction while it determines a classification result.

However, it is possible that each of the classifiers used in acts 308 to 314 are based on different types of models (some examples of which were listed previously) that may be obtained using the training method 400 shown in FIG. 1E. In particular, the training method 400 can be used to determine the best classification model to be used for each of the classifiers used in acts 308 to 314. Each of the classification model parameters can be stored in the data store 252. In at least one embodiment, Matlab-based software and/or Python open source libraries may be used for implementing the classifiers used in acts 308 to 314.

Classifiers that are based on PLSDA, determine a probability of a sample belonging to each class along with a classification threshold. This is done by fitting a Gaussian distribution to all calculated class probabilities.

Classifiers that are based on SVMDA, use a supervised method for classification, regression and outlier detection. The SVMDA is an effective tool in high dimensional spaces. Features are separated in different domains and a probability of each sample belonging to a certain class is calculated. These probabilities are used to estimate the most likely class for each sample (e.g. prediction probability).

Classifiers that use an Artificial Neural Network (ANN) employ a sequential model to build a solution to a problem layer by layer. A “hard sigmoid” activation function may be used for each computation node with binary cross entropy to predict the probability of origin of cancer with, an Adam optimizer algorithm, for example. The cancer origin may be used to determine the cancer type, e.g. lung cancer versus breast cancer. The Adam algorithm utilizes a method of adaptive learning rates and obtains learning rates for individual parameters to optimize the ANN. Alternatively, the ANN may be also be useful, for example, in classifiers that are used to obtain information on the trajectory of cancer prognosis. The ANN may be implemented using python's keras, numpy or sklearn functions, for example.

It should be noted that the method 300 can be performed for each biomarker that is used in performing the cancer assessment which may increase the accuracy of the cancer assessment. Each biomarker will have its own set of classification models. For example, if two biomarkers are used, e.g. a first biomarker and a second biomarker, then there will be a first set of classifiers including a first set having a first cancer type classification model, a first cancer stage classification model, a first metastasis classification model and a first metastasis potential classification model that all correspond to the first biomarker that are used to provide a first set of detection results and then a second set having a second cancer type classification model, a second cancer stage classification model, a second metastasis classification model and a second metastasis potential classification model that all correspond to the second biomarker that are used to provide a second set of detection results. The first and second detection results are then combined at act 316 to provide a more accurate cancer assessment. In at least one embodiment, any combination of the features for cell free DNA, immune biomarkers, exosomes and methylation markers may be used as the training dataset. The combination of biomarkers improves the accuracy of the detection process when compared to using single biomarker. The first and second biomarkers for the classification model can be cell free DNA, immune biomarkers, exosomes or methylation markers.

Accordingly, in at least one embodiment, the cancer assessment at act 316 can be comprehensive depending on the types of biomarkers that are used for performing the method 300. For example, in at least one embodiment, the cancer assessment may include: cancer location, cancer stage, cancer characteristics such as but not limited to metastatic potential, therapy efficacy and the development of adoptive immunity to cancer. Any of the biomarkers described herein can be used to detect these cancer characteristics although the specificity and accuracy can change depending on the particular biomarker used to detect a given cancer characteristic. In at least one embodiment, the comprehensive cancer analysis may include a prognosis for a cancer patient.

Referring now to FIG. 1E, shown therein is a flowchart of an example embodiment of a method 400 for performing training to obtain models that may be used by the software of the computing device 202 in detecting one or more cancer characteristics from the patient sample. For example, the method 400 can be used for obtaining the best model for each of the classifiers that are used in acts 308 to 314 of the cancer assessment method 300. In alternative embodiments, some aspects of the method 400 may be modified.

Acts 402, and 404 are performed in a similar fashion as acts 302 and 304 of method 300. Act 406 involves using PC analysis or MCR analysis, which can be performed as descried previously. However, only one of PC or MCR analysis is performed based on the type of model that is being generated/trained. This may be determined based on the nature of the underlying biomarker for which the model is used.

It should be noted that acts 402 to 406 are performed on several samples to obtain training data which is then used at act 408 for generating/training the classification model. For example, ten or more known cancer samples may be used for training.

Act 408 then involves using the training data from acts 402 to 406. For each sample, there are several features that are used for the training depending on the model structure. For example, when the model is based on PLSDA, the features can be provided by the MCR analysis in which case the first five MCR components can be used.

At act 410, an accuracy assessment of the generated/trained model may be performed, which might be done by running several classifiers in series by applying preprocessing and feature extraction to Raman spectrum of known cancer samples. The classifier giving the highest classification probability is used to determine the type of cancer of the Raman spectrum. This Raman spectra associated with this type of cancer can be removed and the analysis can be repeated for classifying the remaining cancer types.

At act 412, if a number of classification models have been generated/trained then the most accurate one is selected as the optimal model. For example, the model may be further optimized by performing 2,000 iterations for feature extraction and using the features values for the training dataset. Alternatively, a minimum of 10 spectra may be used in the training data for statistical purposes to ensure reproducibility. Each of these spectra were acquired three times and averaged.

### Experimental Results

### Synthesis and Characterization of the Nanosensor

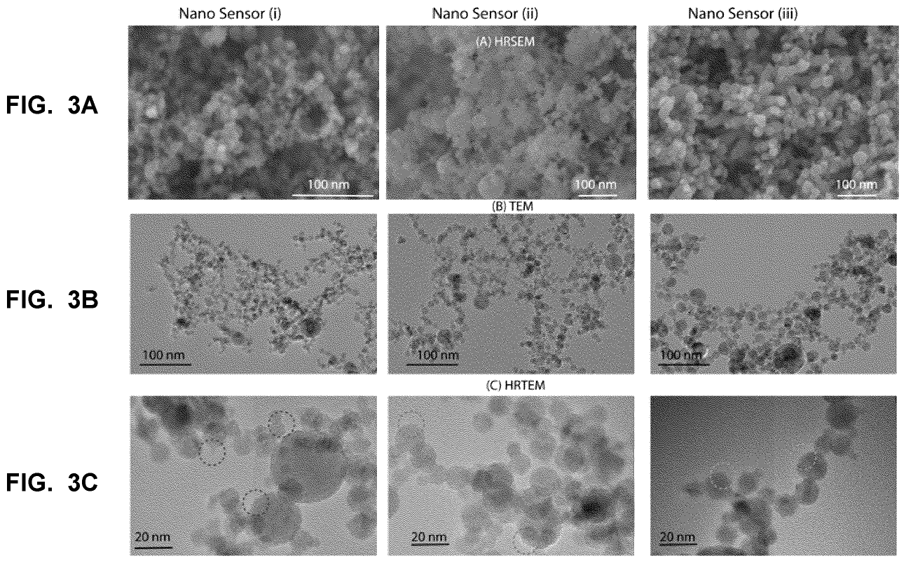

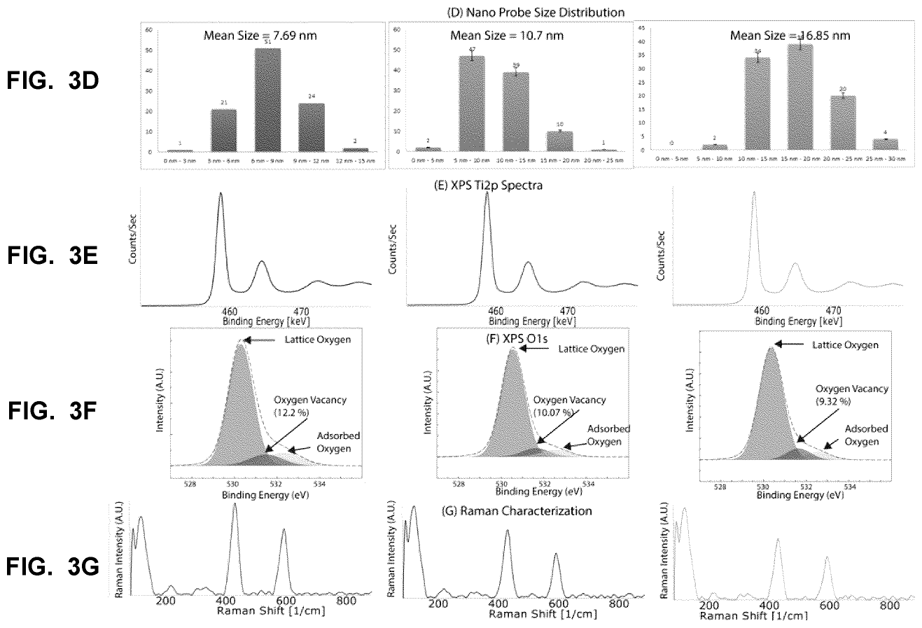

FIG. 3A shows a high-resolution scanning electron microscopy (HRSEM) of a nanosensor according to at least one embodiment demonstrates its morphology. Three dimensional networks of nanoprobe assemblies were evident from HRSEM. This was attributed to the incomplete coalescence during the condensation process in femtosecond laser ablation. The nanoprobes also demonstrated size distribution. As shown in FIG. 3B, HRTEM revealed shape, size and inter planer distance “d”. As shown in FIG. 3C, a spherical shape of the probes was evident from HRTEM images. The spheres were not smooth and irregularities were evident on the surface of the probes. As shown in FIG. 3D, the mean particle size varied from 7.69 nm to 16.85 nm. The particle size increased with increase in the repetition rate. The change in the size of the probes was attributed to the ultrafast fluctuation of temperature of the plasma. When the pulse was “on” more chance of partial coalescence due to the high thermal energy resulted in small sized probes. When the pulse was “off” the ions being less energetic resulted in large sized probes. HRTEM also confirmed polycrystalline structure of the probes.

As shown in FIGS. 3E and 3F, XPS analysis provided information about the surface functionalization of the probes. The Ti 2p spectra showed two characteristic peaks at 549 eV and 464 eV with 5.7 eV separation representing Ti₂p3/2 and Ti₂p1/2, confirming TiO₂ synthesis. The O1S spectrum was fitted into three prominent peaks. The peak at 530.29 eV was assigned to lattice oxygen, the peak at 531.47 eV was assigned to oxygen vacancies and the peak at 532.27 eV was assigned to the adsorbed oxygen. Estimation of oxygen vacancy percentage was calculated by dividing the area under the curve of oxygen vacancies with area under the curve of lattice oxygen. The percentage of oxygen vacancies varied from 12.2% to 9.3%.

Raman spectroscopic analysis was undertaken to investigate the phonon modes of these complex nanosensors. As shown in FIG. 3G, prominent peaks at 138.5 cm-1, signature of Eg peak of anatase TiO₂ mode, 447 cm-1 was assigned to the Eg mode of rutile TiO₂, 604 cm-1 was assigned to A1g mode of rutile TiO₂, a shoulder peak at 630 cm-1 was assigned to the Eg mode of anatase TiO₂ and a broad peak at 235 cm-1 was assigned to the disorder induced scattering or second order effects due to multi-phonon processes were observed. TiO₂ thus, synthesized was hybrid in nature. The intensity of A1g mode reduced with increasing repetition rate demonstrating reduction in oxygen functionalization of the surface with reducing oxygen concentration. Variation in the repetition rate provided control on the peak power or energy transmitted to the sample surface for each pulse. At higher energy transmission, more oxygen functionalization was evident.

The nanosensors were classified on the basis of small probes (mean size 7.69 nm), medium probes (mean size 10.7 nm) and large probes (mean size 16.85 nm).

### Biomarker Detection

As noted above, in at least one embodiment, cell-component biomarkers present in blood plasma include, but are not limited to, extracellular vesicles of circulating cancer initiating cells (CICs). Experiment data for extracellular vesicles of CICs is given below.

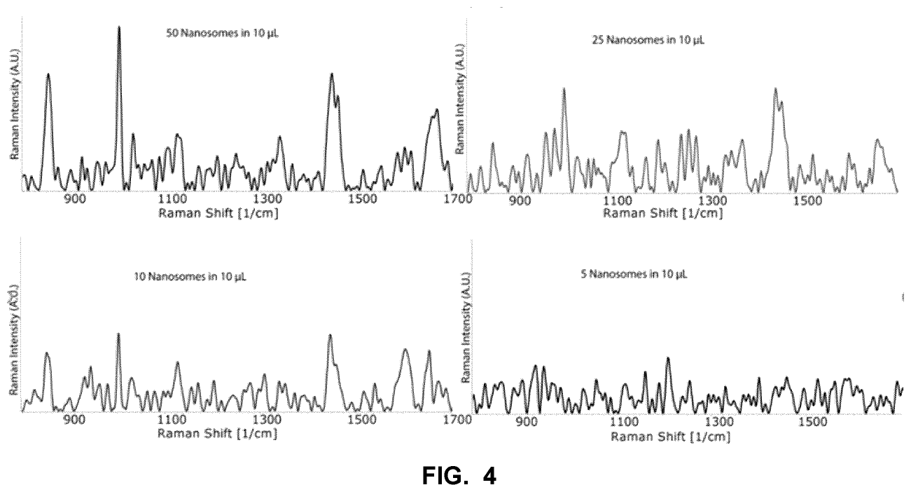

Specifically, SERS profiling of extracellular vesicles of CICs revealed unique features for different types of cancers which was attributed to variation in the contents of the extracellular vesicles of CICs. Unique features derived from SERS profiles enabled prediction of localization of cancer. This approach demonstrated ultra-high sensitivity by trace level detection of up to 10 extracellular vesicles in 10 μl solution. The sensor described herein was also tested with samples derived from clinical specimens of breast cancer cells, lung cancer cells and colorectal cancer cells extracellular vesicles up to 10 extracellular vesicles per 10 microliters was achieved. Specifically, template matching was performed for detection. The signature Raman peaks of the template were visible until 10 extracellular vesicles dilution in 10 microliters. On further dilution, the peaks did not appear. FIG. 4 shows where detection occurred. A location of cancer with 95% specificity was also achieved.

Raman signature peaks of the Raman spectra were used to identify breast cancer (this is used as standard template for breast cancer diagnosis). This is a typical description of a biomolecule (for example, Bovine Serum Albumin (BSA). The Raman assignments were obtained from the literature.

TABLE 1
Raman Assignment for Bovine Serum Albumin (BSA).

| Wavenumber (1/cm) | Raman Assignment |
|---|---|
| 541 | SS stretching |
| 642 | Phe COO— deformation |
| 655 | Phe ring C—C deformation |
| 679 | Trr ring C—C deformation |
| 719 | Tyr |
| 762 | Trp |
| 873 | Tyr |
| 951 | CC stretching |
| 1028 | Phe ring breathing |
| 1069 | Trp |
| 1117 | CN stretching |
| 1173 | Tyr + Phe |
| 1244 | Amide III |
| 1318 | CH2 twisting or CH bending |
| 1350 | Tyr |
| 1447 | CH2 and CH3 scissoring |
| 1511 | Amide II |
| 1616 | Tyr + Phe |
| 2915 | CH stretching, Tyr + Phe |

### Cancer Early Diagnosis

Early cancer diagnosis was shown due to the nanosensor amplifying extremely weak signals of extracellular vesicles of CSCs in circulation. The nanosensor was synthesized with self-assembled, three-dimensional nano-probes that detect biomolecular configuration of extremely low levels of extracellular vesicles associated with CSCs (e.g. 10 extracellular vesicles in 10 μl of solution of spiked samples). The nanoprobes of the nanosensor promote trapping of extracellular vesicles, have an increased surface area, and permit drainage of fluids improving extracellular vesicles-surface interaction. Increased surface area enables improvement in adsorption of the analytes under investigation. By synthesis of the probes at nanoscale, effective surface area increases, which in turn enables more molecules to adsorb resulting in significant amplification of Raman signals. Signature profiles of the extracellular vesicles derived from lung, breast and colorectal CSCs were obtained by a surface-enhanced Raman scattering (SERS) technique. The profile templates were compared with 2 μL plasma samples obtained from lung cancer, breast cancer and colorectal cancer patients (12 each). The extracellular vesicles of CSCs in circulation were shown to act as an independent marker for accurate and early diagnosis. Predictions of the localization of cancer with very high sensitivity (92% to 100%) and specificity (94% to 100%) were made directly from the plasma samples without the need for isolation of extracellular vesicles. The extracellular vesicles of CSCs in circulation demonstrated the potential for early prognosis of cancer in real time. For instance, the method relies on direct detection of extracellular vesicles from plasma (e.g. performing measurements to obtain Raman spectra and then applying template matching to perform the detection), without the need for preprocessing/isolation. In at least one embodiment, each test process took less than about 10 minutes after receiving the plasma samples. Predictions of the location of cancer simultaneously for multiple cancer types were made with plasma samples derived from cancer patients without any need for extracellular vesicles isolation.

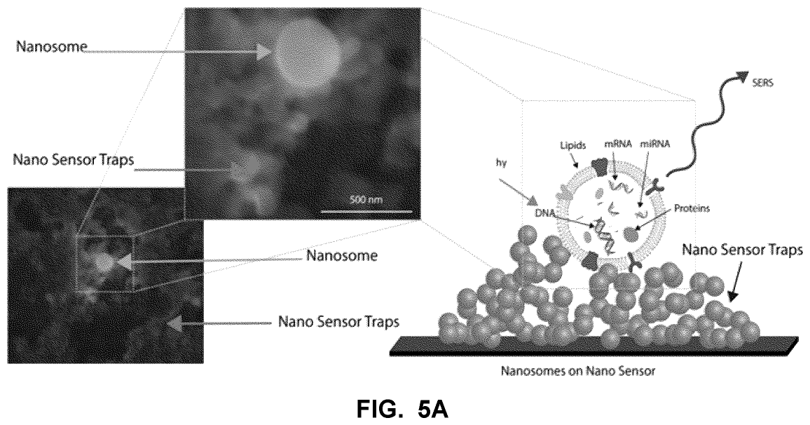

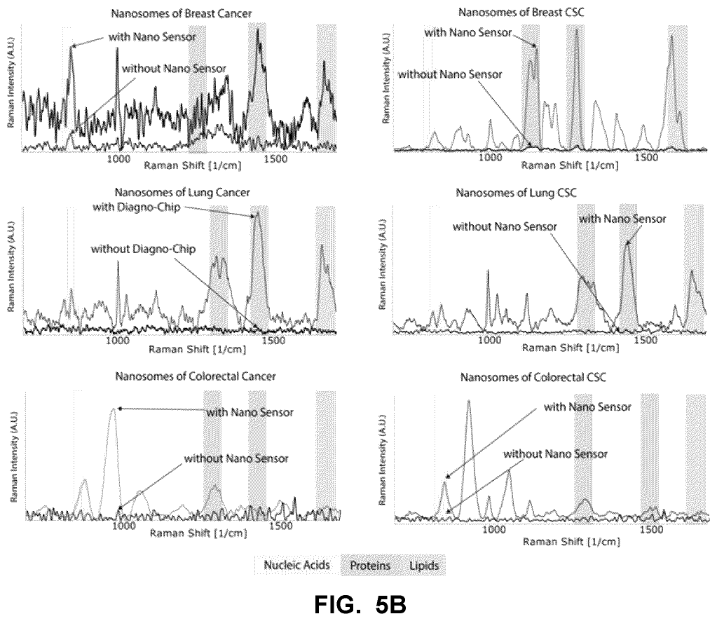

FIGS. 5A-5B show a nanosensor-based SERS signal amplification for circulating extracellular vesicles derived from various types of cells. Specifically, FIG. 5A shows a schematic representation of circulating extracellular vesicles on the nanosensor together with FESEM images of circulating extracellular vesicles on the nanosensor. FIG. 5B shows SERS spectra from circulating extracellular vesicles derived from breast, lung and colorectal cancer and CSCs that were captured by the nanosensor.

Amplification of the Extracellular Vesicle Signatures with Nanosensor

Raman signals of the circulating extracellular vesicles derived from cancer cells and its CSC counterparts demonstrated substantially enhanced signal response. As per FIGS. 5A-5B, all cell lines demonstrated enhanced Raman response for the peaks assigned to nucleic acids, proteins as well as lipids. Circulating extracellular vesicles of lung cancer and lung CSCs demonstrated almost similar signal enhancement trends. Circulating extracellular vesicles of lung cancer demonstrated up to about 20-fold signal enhancement and circulating extracellular vesicles of lung CSCs demonstrated slightly higher signal enhancement, e.g. up to about 23 fold signal enhancement. Circulating extracellular vesicles of breast cancer cells demonstrated up to about 10-fold enhancement whereas circulating extracellular vesicles of breast CSC showed up to about 24-fold enhancement. Circulating extracellular vesicles of colorectal cancer demonstrated about 45-fold enhancement while circulating extracellular vesicles of colorectal CSC showed about 49-fold enhancement. It is evident from the SERS spectra of all cell lines that the extracellular vesicles cargo carried by the cells contained different biomolecular configurations for different cells.

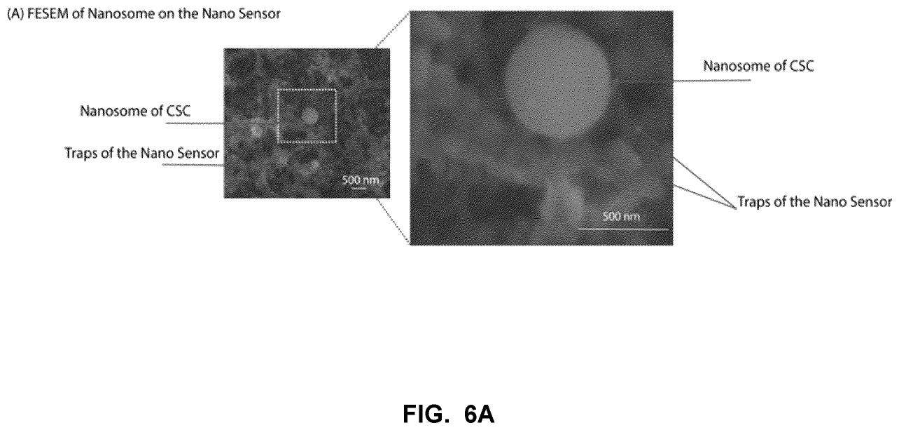

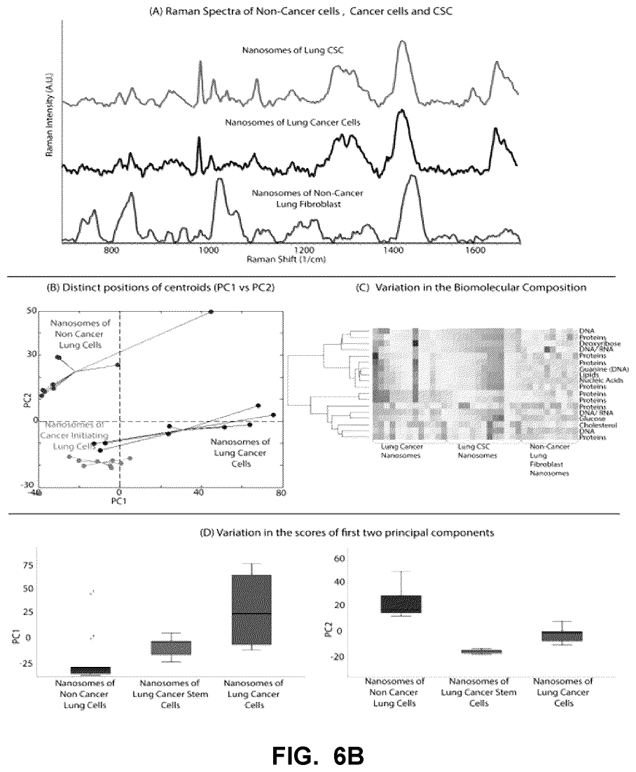

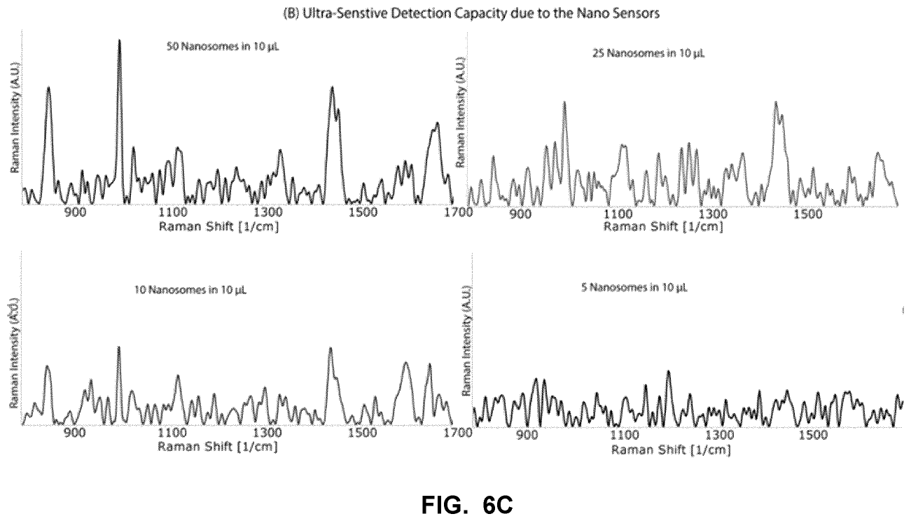

As per FIGS. 6A-6C, the ability of the nanosensor to discriminate between the circulating extracellular vesicles of lung fibroblast (non-cancer cells) from lung cancer cells and lung CSC was evaluated. The nanosensor demonstrated spectral variation in all three types of circulating extracellular vesicles. Principal component analysis demonstrated distinct clustering with centroids in different quadrants. The first two principal components demonstrated significant variation.

Limit of detection of the circulating extracellular vesicles was evaluated to demonstrate whether the technique provided ultra-sensitive detection. The experiment was started with 50 circulating extracellular vesicles in 10 μl of solution and the concentration was reduced to 5 circulating extracellular vesicles in 10 μl of solution. As per FIGS. 6A-6B, detection of circulating extracellular vesicles was achieved up to 10 circulating extracellular vesicles. Characteristic Raman bands were substantially enhanced. For example, the peak at 852 cm⁻¹ representing proline, hydroxyproline and ring breathing mode tyrosine; the peak at 1001 cm⁻¹ representing phenylalanine and reduced nicotinamide dinucleotides (NADH), the peak at 1445 cm⁻¹ representing CH₂ vibration of proteins and CH₂ and CH₃ scissoring of phospholipids, the peak at 1460 cm⁻¹ representing deoxyribose vibration, the peak at 1660 cm⁻¹ representing C═C stretching of lipids, the peak at 1670 cm⁻¹ representing p sheet vibration in amide I, the peak at 2872 cm⁻¹ representing CH₂ symmetric stretch of lipids and CH₂ asymmetric stretch of lipids and proteins, the peak at 2927 cm⁻¹ representing symmetric CH₃ stretch primarily due to proteins and the peak at 2940 cm⁻¹ representing C—H vibrations in lipids and proteins were all substantially enhanced for all concentrations up to 10 circulating extracellular vesicles. The enhancement intensity reduced with the reduced concentration of circulating extracellular vesicles. For example, the intensity of Raman bands did not demonstrate substantial enhancement at the concentration of 5 circulating extracellular vesicles, and the signal was more like noise.

Identification of Circulating Extracellular Vesicles from CSCs

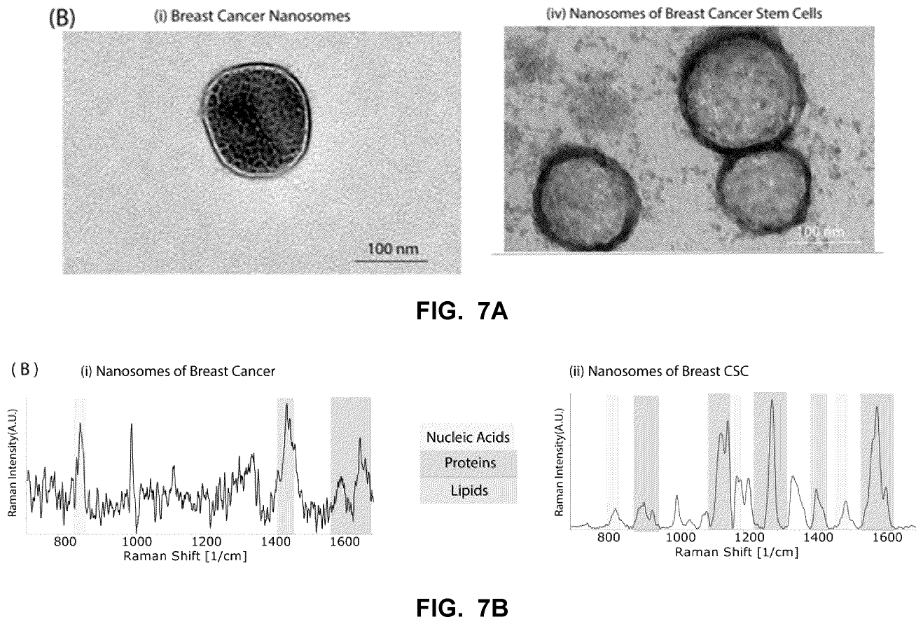

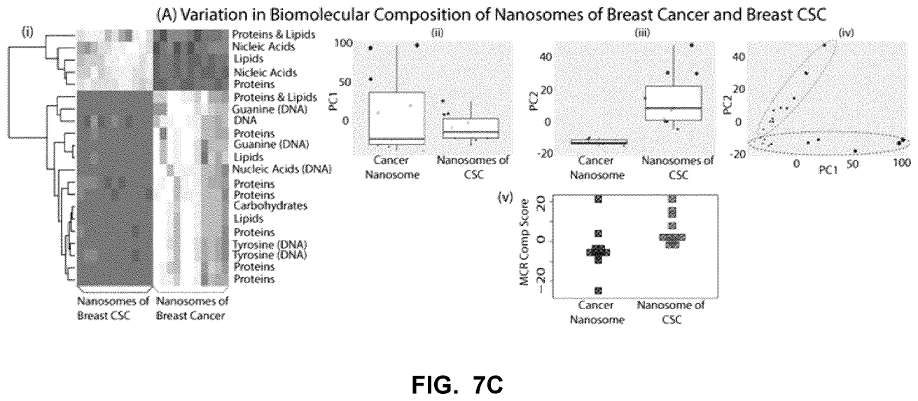

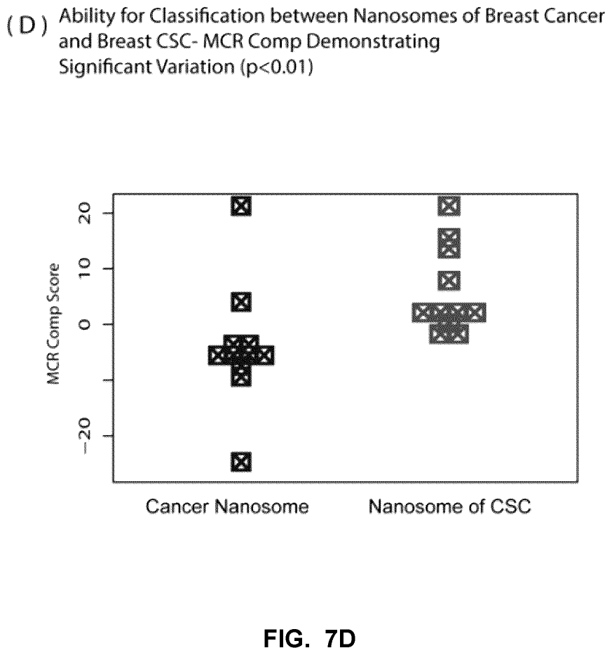

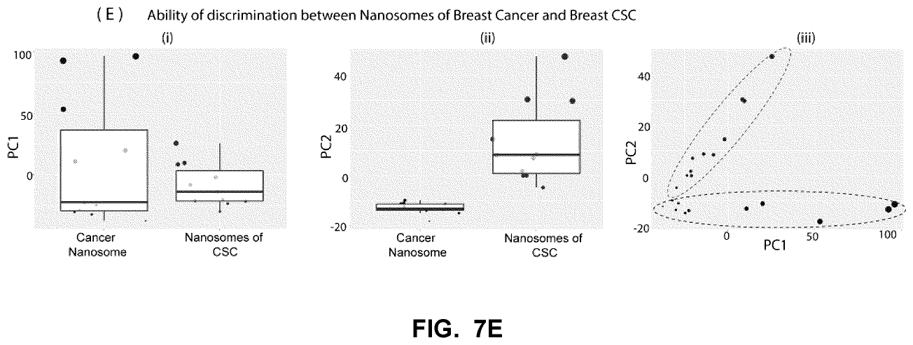

SERS profiling of circulating extracellular vesicles of breast cancer cells as well as circulating extracellular vesicles derived from breast CSC was undertaken. FIG. 7A shows TEM images of the circulating extracellular vesicles. As per FIG. 7B, investigation of the variations in the signatures demonstrated a variation in extracellular vesicles contents. Circulating extracellular vesicles of breast cancer cells demonstrated peaks at 828 cm⁻¹ assigned to O—P—O stretching of DNA, 854 cm⁻¹ assigned to tyrosine ring breathing mode, 1001 cm⁻¹ assigned to proteins, 1353 cm⁻¹ assigned to proteins, 1448 cm⁻¹ assigned to lipids, 1603 cm⁻¹ assigned to COO stretch in proteins, and 1660 cm⁻¹ assigned to lipids. Circulating extracellular vesicles of breast CSC showed multiple additional peaks including peaks at 746 cm⁻¹ assigned to ring breathing mode of DNA/RNA bases, 828 cm⁻¹ assigned to O—P—O stretching of DNA, 896 cm⁻¹ assigned to deoxyribose vibrations, 935 cm⁻¹ assigned to proline and hydroxyproline, 1007 cm⁻¹ assigned to proteins, 1133 cm⁻¹ assigned to proteins and lipids, 1155 cm⁻¹ assigned to C—N stretch in glucose, 1181 cm⁻¹ assigned to proteins, 1215 cm⁻¹ assigned to collagen, 1343 cm⁻¹ assigned to proteins and lipids, 1496 cm⁻¹ assigned to lipids, 1586 cm⁻¹ assigned to Amide I band in proteins and 1614 cm⁻¹ assigned to C═O carbonyl stretch in proteins. As per FIG. 7C, the heatmap of the biomolecular Raman intensities demonstrated downregulation of the bands assigned mostly to proteins and lipids and nucleic acids whereas increase in tyrosine and guanine modes of DNA, carbohydrates and proteins in circulating extracellular vesicles of CSCs as compared to circulating extracellular vesicles from breast cancer cells.

To visualize the variation in the contents of circulating extracellular vesicles, multivariate analyses (principal component analysis PCA and multivariate curve resolution (MCR) analysis were undertaken. PCA is a technique that can be used to analyse data from multiple inter-correlated variables. As shown in FIG. 7E, a plot of principle component PC1 as well as principle component PC2 in PCA showed statistical significance (p<0.05). On plotting PC1 versus PC2, two separate clusters of circulating extracellular vesicles derived from breast cancer and breast CSCs were visible. As per FIG. 7D, the bee swarm plot of MCR component also demonstrated a significant difference (p<0.01).

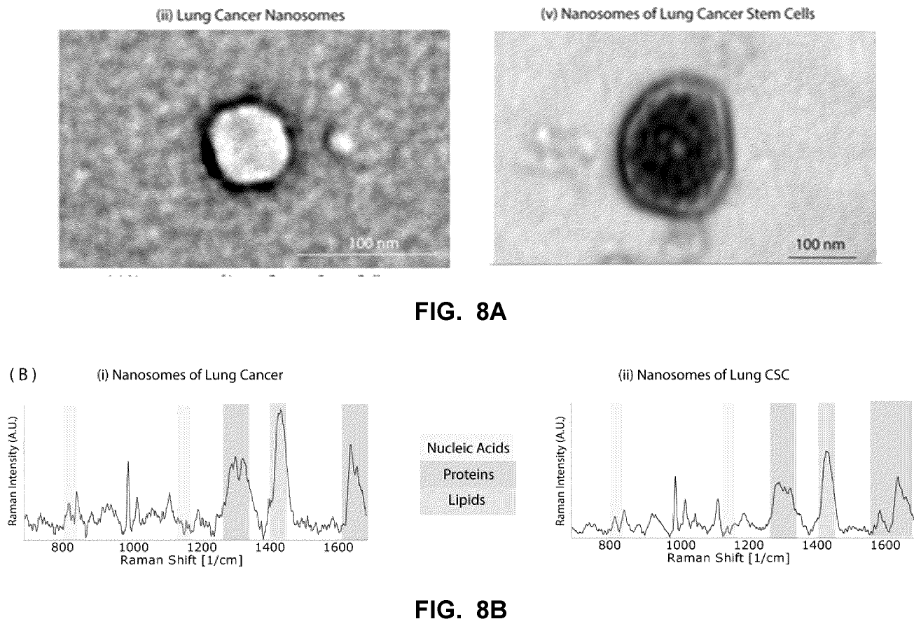

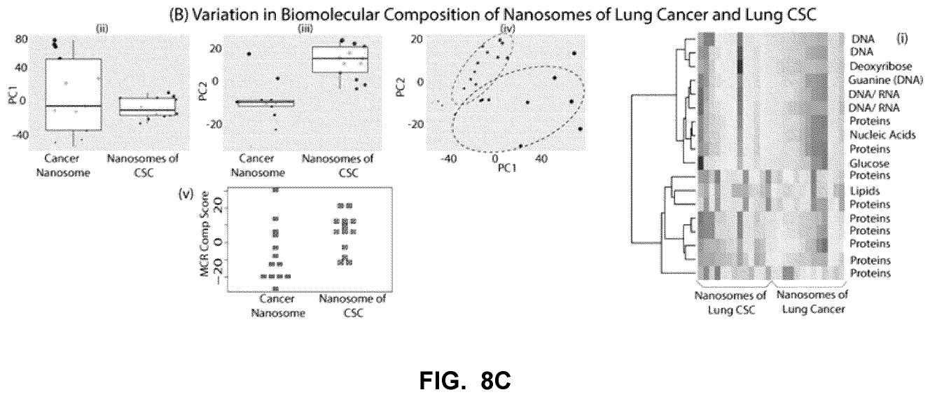

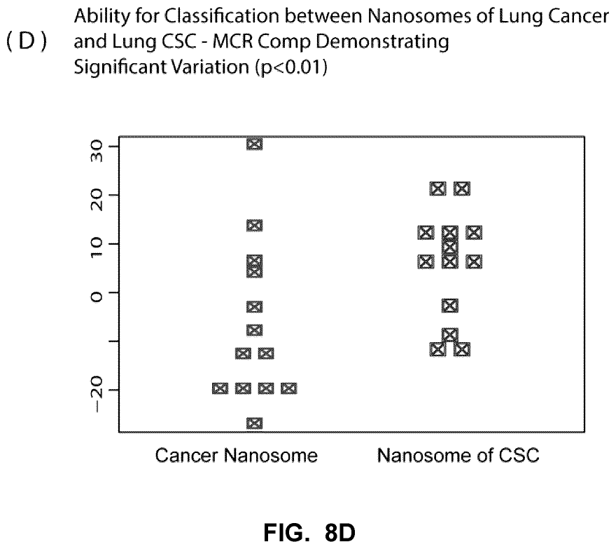

A similar trend was also observed with the circulating extracellular vesicles of lung cancer cells and circulating extracellular vesicles of lung CSCs. FIG. 8A demonstrates TEM images of circulating extracellular vesicles of lung cancer cells and lung CSC. As per FIG. 8B, variation in the peak intensity for the peaks at 743 cm⁻¹ assigned to ring breathing mode of DNA/RNA bases, 828 cm⁻¹ assigned to O—P—O ring breathing of DNA, 850 cm⁻¹ assigned to Proline, hydroxyproline, 933 cm⁻¹ assigned to (C—C) skeletal stretching of collagen backbone, 1001 cm⁻¹ assigned to proteins, 1030 cm⁻¹ assigned to C—N stretching of proteins, 1126 cm⁻¹ assigned to proteins and lipids, 1206 cm⁻¹ assigned to nucleic acids, 1305 cm⁻¹ assigned to CH₂ deformation in lipids, 1338 cm⁻¹ assigned to ring breathing modes in the DNA bases, 1445 cm⁻¹ assigned to lipids, 1600 cm⁻¹ assigned to C═O stretching in amide I band and 1652 cm⁻¹ assigned to carbonyl stretch of proteins was observed. As per FIG. 8C, the heat map demonstrated downregulation of mostly nucleic acids in circulating extracellular vesicles of lung CSC as compared to the circulating extracellular vesicles of lung cancer cells. Upregulation of proteins and lipids in circulating extracellular vesicles of lung CSC was also visible from the heatmap. As per FIG. 8E, a plot of PC2 demonstrated statistical significance and clear clustering in the plot of PC1 versus PC2 was evident. Similar to the breast CSC analysis, as per FIG. 8D, significant variation in the MCR component was also evident for the circulating extracellular vesicles of lung cancer vs lung CSC (p<0.01).

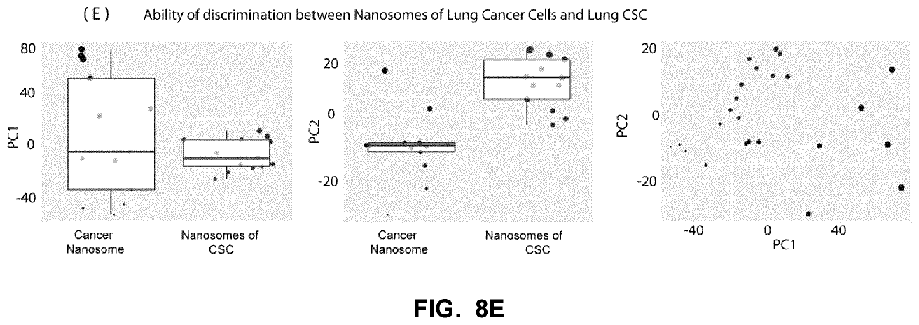

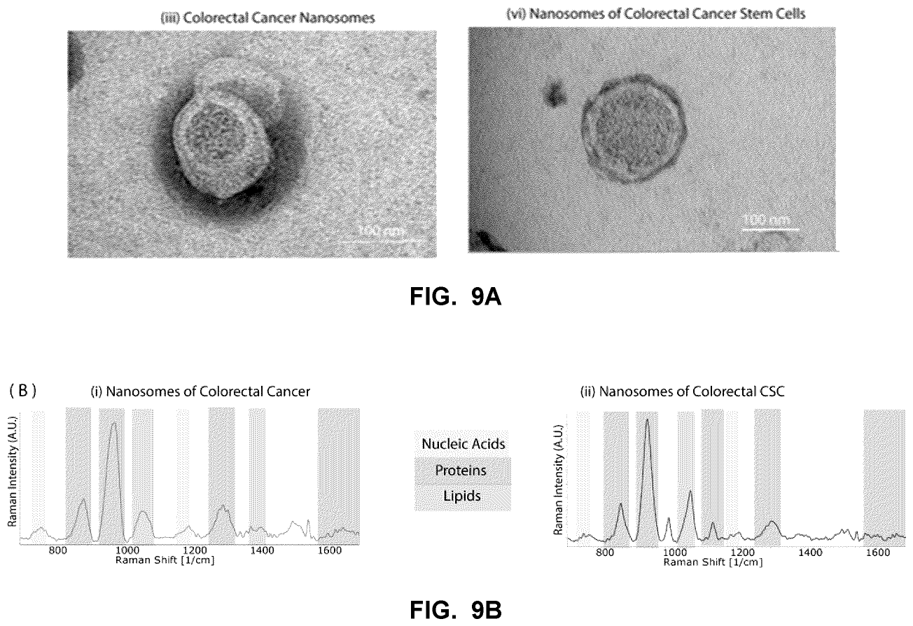

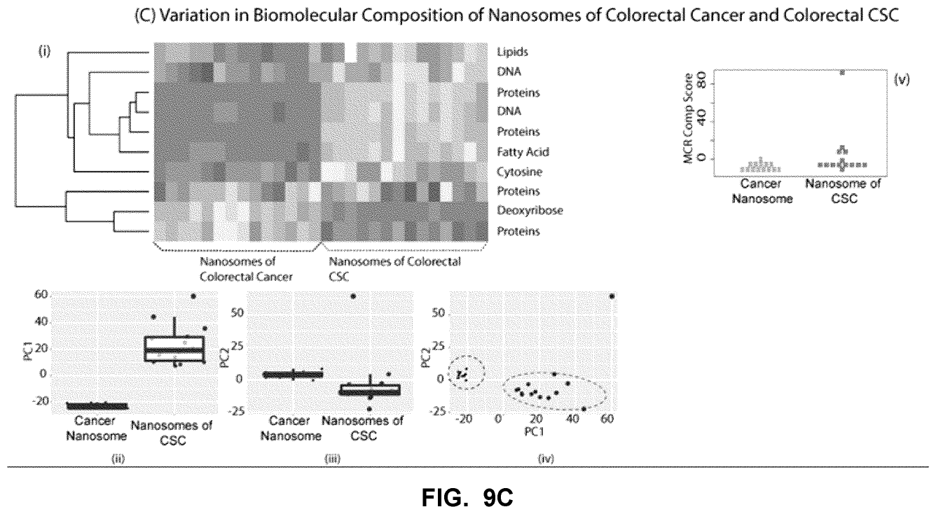

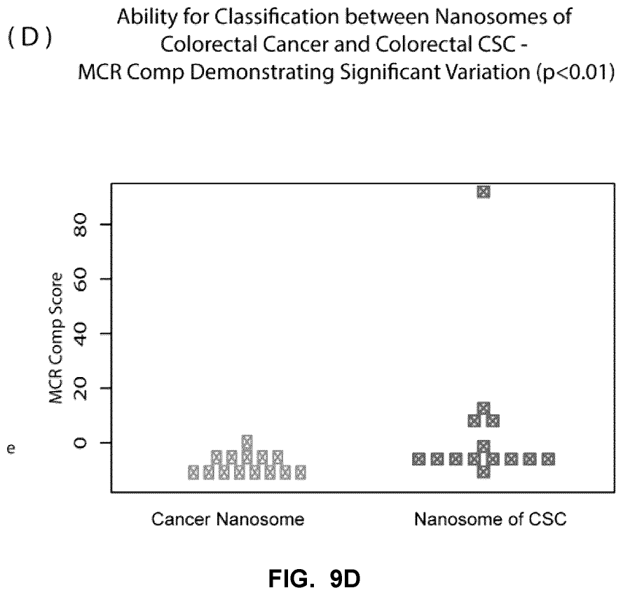

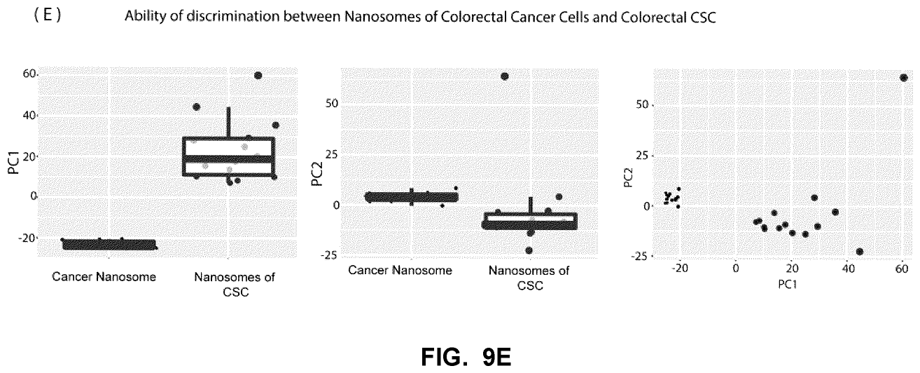

FIG. 9A shows TEM images of the circulating extracellular vesicles of colorectal cancer cells and CSCs. As per FIG. 9B, signature peaks for biomolecules such as proteins, fatty acids, cytosine, DNA and lipids were evident for both types of cancers. For colorectal cancer, many Raman assignments of lipids, proteins, DNA and nucleic acids demonstrated upregulation in the circulating extracellular vesicles of colorectal CSCs as compared to colorectal cancer cells. This was evident from the heat maps plotted in FIG. 9C. The bee swarm plot of MCR component score demonstrated significant variation as shown in FIG. 9D. As per FIG. 9E, the first two principal components in principal component analysis demonstrated significant variation and clear clustering.

The experimental results from circulating extracellular vesicles derived from cancer cells versus cancer CSC clearly demonstrated that the nanosomal cargo (i.e. extracellular vesicles) were significantly different. Extracellular vesicles (e.g. exosomes) contain many biomolecules such as proteins, lipids, nucleic acids and other cellular components that may contain important information about parent cells. These biomolecules are carried by exosomes are of importance in intracellular communication. These contents are called exosomal cargo. The cargo may vary based on physiological and pathological; conditions, which is confirmed by this study. As our experiments clearly indicate that Raman profile of extracellular vesicles of cancer and CSC show variation in the Raman bands representing nanosomal cargo.

Nanosensor Assisted Cancer Prognosis of the Localization of Cancer from Circulating Extracellular Vesicles of CSC

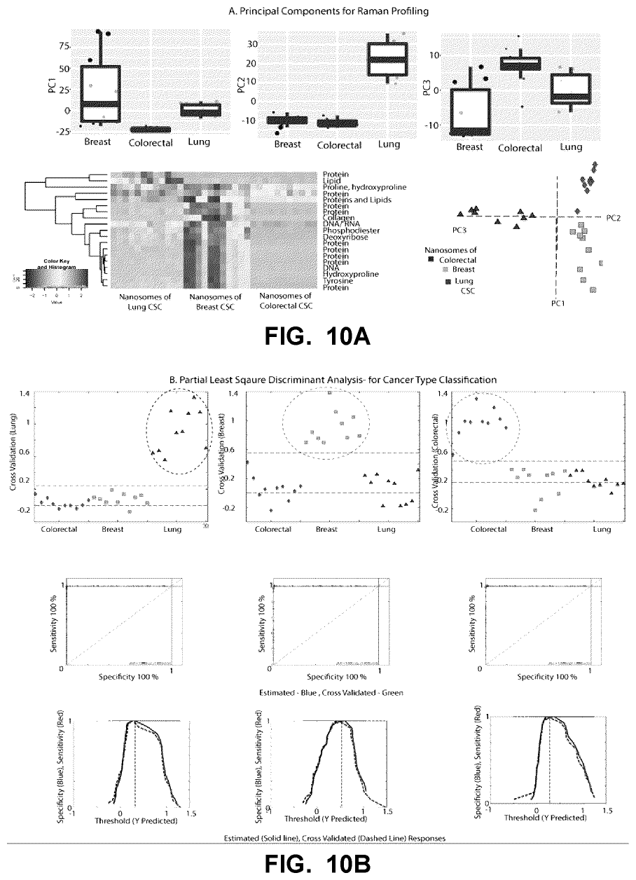

The performance of nanosensor assisted SERS profiling of the circulating extracellular vesicles derived from three types of CSCs (breast, lung and colorectal) was evaluated. As per FIG. 10A, principal component analysis demonstrated significant variation in the scores of principal component 1 and principal component 3. Principal components are derived by reduction in the dimensionality of data. These components signify the difference between the extracellular vesicles.

When the scatter plot of PC1 vs PC2 is plotted, and if it shows two distinct clusters then it means that two different types of extracellular vesicles were detected. Sometimes a three-dimensional scatter plot of PC1 vs PC2 vs PC3 is also plotted for better visualization.

The three-dimensional plot of PC1 vs PC2 vs PC3 demonstrated three separate clusters. Signature peak intensities were plotted in a heat map to visualize variation in the nanosomal contents. Circulating extracellular vesicles of breast CSC demonstrated much higher expression of almost all biomolecules and low expression of a couple of proteins and lipids as compared to the Circulating extracellular vesicles of the other two types of CSCs. Circulating extracellular vesicles of lung CSC demonstrated very high expression of proteins and lipids and very low expression of proline, hydroxyproline and proteins. Circulating extracellular vesicles of colorectal CSC demonstrated high expression of proline, hydroxyproline and proteins and moderate expression of other biomolecules.

Prediction of the location of the cancer was attempted with nano sensor based analysis of circulating extracellular vesicles of CSCs. For this purpose, a hierarchical model was developed. First, MCR analysis was undertaken.

As shown in FIG. 10B, the classification of the circulating extracellular vesicles of colorectal, lung and breast cancer cells were classified with the training model with 100% prediction accuracy. ROC curve (blue for estimated and green for cross validated) perfectly match each other. Cross validation showed 0% false positive or false negative errors. Misclassification error was as little as 4% for circulating extracellular vesicles of breast CSC, 4.5% for circulating extracellular vesicles of lung CSC and 0% for circulating extracellular vesicles of colorectal CSC. The nano sensor-based prediction model designed with the circulating extracellular vesicles of CSC demonstrated very high sensitivity as well as specificity.

Specifically, the model demonstrated 100% sensitivity and 98.46% specificity for lung cancer cross validation results and 100% sensitivity and 100% specificity for breast and colorectal cancer cross validation. For prediction with patient plasma, the model was applied in two stages, as follows.

First, lung CSC extracellular vesicles were treated as one class and remaining breast and colorectal cancer extracellular vesicles were treated as one class. Using PLSDA, the classification showed 83.33% sensitivity and 91.66% specificity of classification to predict lung cancer tissue of origin.

TABLE 2
Confusion Table

| | Class O | Actual Class Lung |
|---|---|---|
| Predicted as Class O | 22 | 2 |
| (Breast cancer + Colorectal Cancer) | | |
| Predicted as Lung Cancer | 2 | 10 |
| Predicted as Unassigned | 0 | 0 |

Mathew's correlation coefficient = 0.750

In the second stage, breast cancer and colorectal cancer patient plasma were classified. Prediction of tissue of origin as breast cancer classification showed 90.90% sensitivity and 100% specificity. Prediction of tissue of origin as colorectal cancer showed 100% sensitivity and 90.91% specificity.

TABLE 3
Confusion Table

| | Actual Class | |
|---|---|---|
| | Breast Cancer | Colorectal Cancer |
| Predicted as B | 10 | 0 |
| Predicted as C | 1 | 12 |
| Predicted as Unassigned | 0 | 0 |

Mathew's correlation coefficient 0.921

Applicability of Plasma without Enrichment or Extracellular Vesicles Isolation for Rapid Diagnosis with the Nanosensor Assisted Prognosis

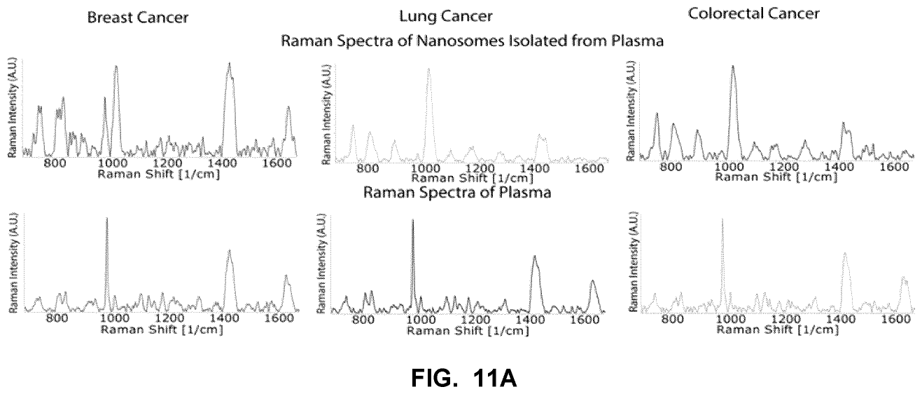

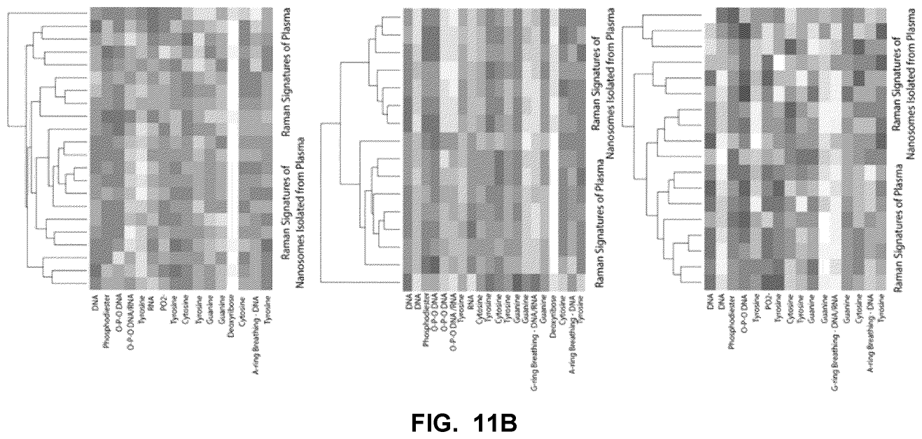

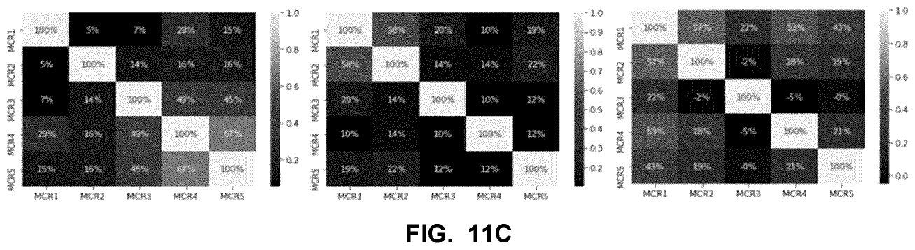

As shown in FIG. 11, the Raman spectra of plasma and extracellular vesicles isolated from plasma were compared. Isolation of exosomes from plasma was done using Plasma exosome isolation kit (Cusbio-Catalog No, CSB-E10102) using the manufacturer's protocol. Briefly, plasma was centrifuged at 10000 rpm for 10 minutes and supernatant was retained. The supernatant was filtered through 0.45 μm filter and using the reagents from the kit, centrifuging at 850 rpm for 1 minute, mixing with buffer and eluting at room temp for 10 minutes, final isolated exosomes were retained. Stored at 4° C. for short term usage.

For visualization of signature Raman assignments, a heat map of signature bands was plotted. The similarity of the Raman profiles was evident from the spectra as well as the heat maps. MCR was undertaken to solve the problem of mixtures. MCR analysis results in pure components of the mixture. First, five MCR components were compared for similarities and variations by applying an artificial neural network-based model. ANN is one of a variety of different ways of using machine learning. ANN is a brain inspired system intended to learn patterns that are too complex (e.g. spectral data patterns) to extract and teach the machine to recognize. Layer by layer, ANN extracts features of spectra to identify classifying parameters. It should be understood that the feature extraction described herein is not limited to ANN. Rather, any standard deep learning/machine learning methodology/algorithm can be alternatively applied for feature extraction. FIG. 11C shows similarity if the variation is less than 45%, which is used as a threshold to show that the components show correlation. As per FIG. 11, it was evident that plasma and isolated extracellular vesicles demonstrated very similar MCR components.

Plasma samples from breast, lung and colorectal cancer patients were then tested with the cancer diagnosis-chip for localization of cancer. This was achieved by building a hierarchical support vector machine discriminant analysis (SVMDA) and partial least square discriminant analysis (PLSDA) model with training algorithm designed based on Raman spectral features of the extracellular vesicles of CSC. However, again, it should be understood that the feature extraction is not limited to SVMDA, since any standard deep learning/machine learning methodology/algorithm can be alternatively applied for feature extraction.

Specifically, the SVMDA was applied by calling an SVMDA module (e.g. function) in a Matlab-based software. The model details are as follows: SVM type: C-SVC; and SVM kernel type: radial basis function.

SVM demonstrated specificity of 95.8% for detection of lung cancer and 83.3% sensitivity.

TABLE 4
Confusion table

| | Actual Class | Class O Lung |
|---|---|---|
| Predicted as Class O | 25 | 0 |
| (Breast cancer and colorectal cancer) | | |
| Predicted as Lung cancer | 0 | 12 |
| Predicted as Unassigned | 0 | 0 |

In a second stage, breast cancer and colorectal cancer patient plasma was classified. Prediction of tissue of origin as breast cancer classification showed 90.90% sensitivity and 100% specificity. Prediction of tissue of origin as colorectal cancer showed 100% sensitivity and 90.91% specificity.

TABLE 5
Confusion table

| | Actual Class | |
|---|---|---|
| | Breast Cancer | Colorectal Cancer |
| Predicted as B | 10 | 0 |
| Predicted as C | 1 | 14 |
| Predicted as Unassigned | 0 | 0 |

Mathew's correlation coefficient: 0.921

Prediction of Localization of Tumor with Clinical Plasma Samples

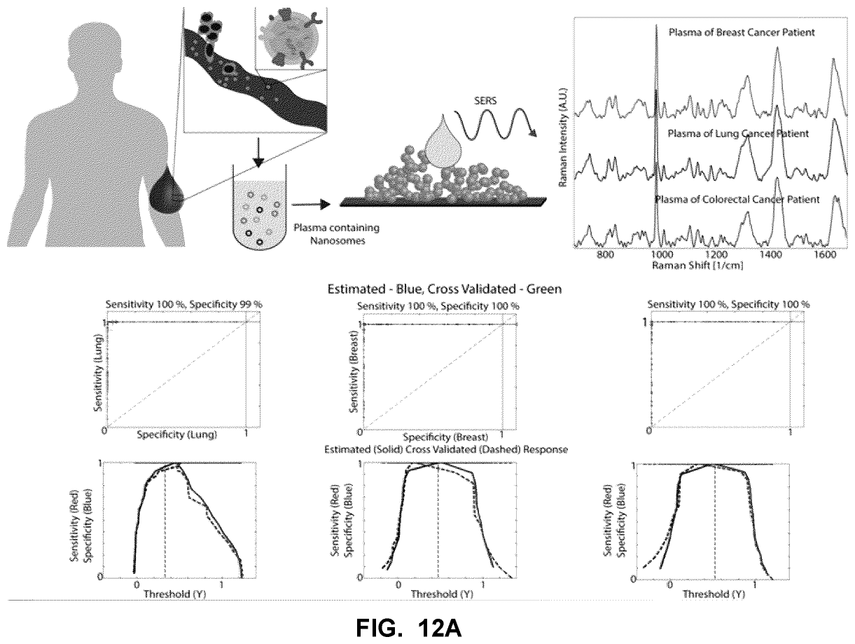

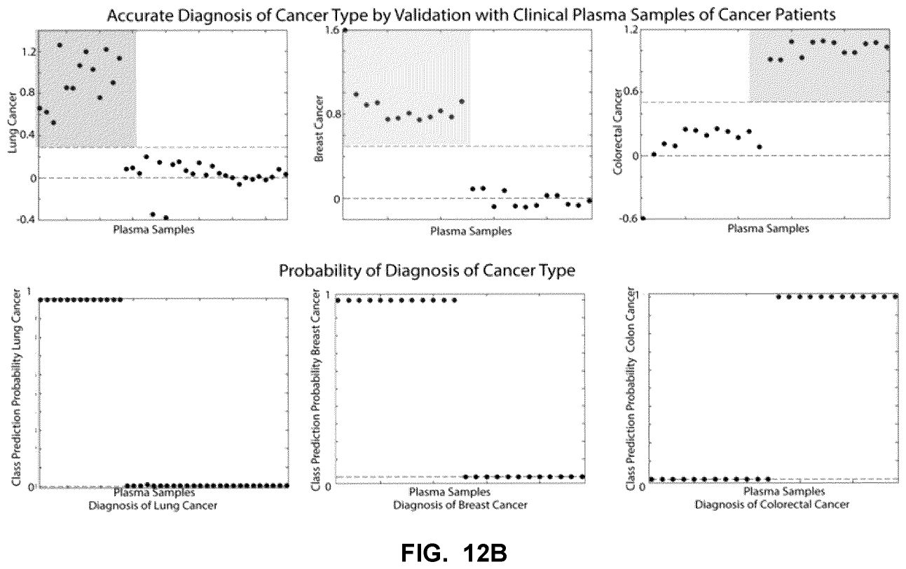

As shown in FIG. 12, the plasma of lung cancer patients was treated as one class while the plasma of breast cancer patients and colorectal cancer patients together was treated as another class in SVMDA. Classification between the two classes was achieved with 92% sensitivity and 94% specificity. The Wilcoxon permutation test demonstrated significant results (p<0.05). After the separation of lung cancer from the mixture, the remaining samples were treated as two separate classes (i.e. plasma of breast cancer patients as one class and plasma of colorectal cancer patients as another class). The latent variables derived from the SVMDA analysis were used for this classification. 100% sensitivity and 100% specificity was achieved with Mathew's correlation coefficient 0.921. These results demonstrated an ability of the nanosensor to provide accurate localization of cancer directly from plasma. This was achieved by analysis of Raman profiles of extracellular vesicles of cancer initiating cells and comparing it, using hierarchical analysis using MCR and PLSDA, directly with plasma.

### Cancer Onset

The following biomarkers may be able to be used to screen cancer onset before any symptoms occur:
i. Concentration of cfDNA: when cancer cells start to accumulate in a certain organ, the cells release several molecules including the cell free DNA, and exosomes into the circulation. When the tumor size is small or comprises only a few thousand cells, the concentration of cfDNA is in the femtomolar concentration range, at which it is virtually undetectable through conventional methods. The 3D nanosensors described herein have the potential to monitor changes in the concentration of cfDNA directly from plasma. For example, 10 μg of plasma solution was dropped on the nanoprobes and the substrate without nanoprobes. Raman spectra of the samples were taken after the solution dried. A Renishaw inVia Raman microscope was used. A calibration curve based on limit of detection for various cellular components was compared with plasma peaks to determine cfDNA concentration. The 3D nanosensors provide an amplified signal of the cfDNA. The intensity of amplified signals is directly proportional to the concentration of cfDNA. Hence, by monitoring the changes in the intensity the nanosensor enables the monitoring of the changes in cfDNA.
ii. Methylation of cfDNA: The aberrant methylation of cfDNA is a consistent marker for the origin/onset of cancer. Further, it is present throughout the cancer evolution process, making it an ideal marker for onset, diagnosis, and prognosis of cancer.
iii. Exosomes: Exosomes are valuable resources for cancer onset detection because of their ability for regulating the tumour microenvironment, and for selective cargo loading, as well as their high similarity to the cells from which they are secreted. The exosomes have information regarding the onset of cancer, the cell-cell communication leading to cancer growth and the origin of cancer.

By combining the epigenetic profile of cfDNA and the epigenetic and proteomic profiling of exosomes, it is possible to accurately screen the onset of cancer in a healthy population.

The methodology for determining cancer onset, in accordance with the teachings herein, involves establishing a spectroscopic template for differential methylation percentage and validation using the cell culture derived DNA. The template may be further applied to DNA isolated from the solid tumor and the corresponding ctDNA, which will confirm that ctDNA methylation is correlated. The healthy plasma is then spiked with cell culture derived DNA to analyse the limit of detection in a complex sample. The spectroscopic data may then be subjected to multivariate analysis to investigate the diagnostic sensitivity, specificity, and analytical limit of detection of the proposed method. Finally, the plasma samples may be analysed using the multivariate analysis components for the diagnosis, prognosis and prediction of cancer metastasis using ctDNA methylation patterns, cfDNA concentration and the exosome concentration.

### Metastatic Cancer Detection

The application of three-dimensional nanosensors amplify the signals associated with circulating tumor DNA (ctDNA) methylation, thereby enabling direct detection from plasma. The main problem associated with utilizing ctDNA in a clinical setting, is their presence in low concentration. The three-dimensional nanosensors possess a single molecular sensitivity which is ideal for the amplification of differentially methylated sites in ctDNA (see FIG. 1). The detection of gene methylation patterns in ctDNA presents a unique potential for prediction of metastasis and the tissue of possible metastasis.

The following biomarkers can identify the probability of a primary tumor to metastasize to distant organs and predict the organ of metastasis. Additionally, these markers can be utilized to detect the presence of metastasis as low as 1,000 cells and pinpoint the origin of metastasis with a high specificity.
i. Methylation of ctDNA: Aberrant methylation of cell free DNA occurs in the early stages of cancer initiation and is present throughout the malignancy. Hence, it can be used as a robust marker for diagnosis, prognosis, and prediction of cancer metastasis. The tumor mutational burden, mutant allele fraction and genetic phenotype of certain cancers are prone to overlapping, thereby reducing the specificity of the molecular diagnosis. However, the methylation pattern is different for each cancer type and tissue of origin. Also, the methylation of certain genes in the cfDNA is associated with initiation of metastasis. Since the current methods to investigate methylation patterns needs cfDNA isolated from the plasma, some of the key information is lost, thereby making the diagnostic process inefficient.
ii. Exosomes: Ultra-sensitive detection ability is necessary in detection of the onset of metastasis, as the biomarkers (proteins in this case) are at trace levels during the initial stage of metastasis. The three-dimensional networks of the nano sensors may act as a trapping mechanism for the exosomes and provide a large surface area that is beneficial for efficient adsorption of plasma proteins. Simultaneously, the nanosensors demonstrated the ability to amplify very weak Raman fingerprints of the biomarkers of interest. Amplification was determined by dropping 10 μg of the solution on the nanoprobes and the substrate without nanoprobes. Raman spectra of the samples were taken after the solution dried. A Renishaw inVia Raman microscope with the following parameters was used
   HC plan APO lenses
   matched polarizer/analyzer optics (20× magnification)
   spot size of 0.625 μm radius
   focal length of 250 mm
   solid state laser (wavelength 785 nm (12.5 mW))
   spectral resolution of 0.5 cm-1 in visible and 1 cm-1 in NUV and IR
   spatial resolution: <1 μm (lateral), <2 μm (depth)
For example proteins associated with the metastatic phenotype (Osteopontin OPN and extracellular matrix protein 1 ECM1) and proteins associated with the non-metastatic phenotype (annexin 1 and matrix metalloproteinase 1 MMP1) have been identified via a literature review). Osteopontin (OPN)—glycosylated phosphoprotein can be found in all body fluids. OPN acts as cell attachment protein as well as signal delivery protein. Poor prognosis has been reported with elevated levels of OPN. High levels of OPN are also associated with malignancy as well as metastasis. High levels of ECM1 are closely correlated to metastasis in many tumor types. Non-metastatic proteins such as annexin 1 are known to regulate phagocytosis, cell signalling as well as cellular proliferation. Therefore, high expression of annexin 1 can be correlated to absence of metastasis. MMP proteins are responsible for remodeling of extracellular matrix, embryonic development as well as tissue remodeling. Expression of polymorphism in MMP1 and cancer risk has already been correlated.
iii. Immune cells—T cells: During each step of the metastatic cascade, cancer cells tend to mutate and therefore potentially immunogenic cancer cells can be recognized and killed by the host immune system. However, cancer cells exploit several mechanisms to evade destruction by the immune system, enabling them to proceed through the metastatic cascade. Additionally, under certain circumstances some immune cells and their mediators in fact favor metastatic disease and tumor growth. Based on the above-mentioned criteria, the immune cells have the potential to serve as both diagnostic and prognostic markers for cancer metastasis. The immune landscape is constantly changing which can be utilized to study the evolution of cancer. In accordance with the teachings herein, a non-invasive method is provided to diagnose cancer metastasis by studying the intracellular changes in the immune cells such as T− cells, NK cells, myeloid derived suppressor cells. T cells express cancer specific antigens, which are useful in cancer detection. In addition, the ratio of CD 4+ T cells to CD 8+ T cells shows the immune status of the individual. The presence of CD4+ cells is indicative of immune suppression which leads to metastasis. Further, the presence of tumor-infiltrating CD8+ T cells and Th-1 cytokines in tumors correlates with a favorable prognosis in terms of overall survival and a disease-free survival in many malignancies. By utilizing the above mentioned factors, a new marker for cancer metastasis is established by detecting the changes in the immune landscape non-invasively.

In accordance with the teachings herein, a method is provided that involves establishing a spectroscopic template for differential methylation percentage and validation using cell culture derived DNA. The template may further be applied to DNA isolated from solid tumor and the corresponding ctDNA, which may confirm that ctDNA methylation is correlated with tumor stage, tumor size and metastasis. The spectroscopic data may then be subjected to multivariate analysis to investigate the diagnostic sensitivity, specificity, analytical limit of detection of the proposed method. Finally, the plasma samples may be analyzed using the multivariate analysis components for the diagnosis, prognosis and prediction of cancer metastasis using ctDNA methylation patterns.

### Quantum Superstructure-Based Liquid Profiling: Mapping of Circulating DNA Structure for Cancer (MCSC)

One hallmark of cancer is the aberrant changes in the DNA structure. DNA damage is prominently co-related to carcinogenesis. The damage to DNA is associated with reactive Oxygen species (ROS) following which DNA conformational changes occur. Such changes at nanometer scale are extremely difficult to diagnose without any artificial amplification of DNA. However, artificial amplification can lead to introduction of spectral artefacts. Therefore, quantum superstructure assisted profiling of the DNA structural changes in cancer was undertaken without need for DNA amplification. Raman profiling of these changes will lead to timely identification of the disease. 10 μL of DNA solution was added directly to the quantum probes and Raman spectra were obtained from Breast, Lung and Colorectal cancer cells as well as from DNA of its CSCs.

Raman bands in the range of 600 cm-1 to 700 cm-1 are assigned to the sugar-base conformation dependent ribose vibration of Guanine. The orientation sensitive band at 685 is sensitive to was shifted to the lower wavenumber for DNA of cancer cells as well as CSCs. The peak shift was attributed to the disruption in base-stacking resulting in the deformation around lesion sites in cancer. The bands in the region of 800 cm-1 to 1100 cm-1 demonstrate sensitivity to the secondary DNA structure as well as geometry of backbone. The band assigned to phosphodiester backbone and deoxyribose, which is located at 890 cm-1 was shifted to 875 cm-1 for breast CSC, 870 cm-1 for lung CSC. For cancer cells, it showed a slight shift to 875 cm-1 for colorectal cancer, 884 cm-1 for lung cancer and did not show any shift for breast cancer. The shift was attributed to the scissions of DNA, more scission for CSCs as compared to its cancer counterpart was observed. Intensity of this peak was much higher for CSC DNA as compared to cancer cell DNA. This was attributed to the unstacking of DNA bases in CSCs. This was also supported by the substantially increased intensity for the band at 789 cm-1 (C<T and backbone). This could be because of the un-pairing of the paired C, T in CSCs. Opposite trend was observed for the band at 1333 cm-1 (assigned to A,G). The intensity of this band was increased substantially for cancer cell DNA as compared to CSC DNA demonstrating structural damage in cancer cells. The band at 1085 cm-1 was observed to be shifted towards a lower wavenumber for all types of DNAs, indicating ROS induced backbone damage. This damage was much higher in cancer cells as compared to CSCs. The ratio of the peak intensity at 1387 cm-1 and 1334 cm-1 can provide information about DNA aggregation. It was observed that DNA from CSCs showed much higher value of this ratio as compared to cancer cells. This is an indication of the perturbation of the local environment around purine bases in CSCs.

Comprehensive MCSC Analysis of CSC DNA and Cancer Cell DNA Assisted with Quantum Superstructures

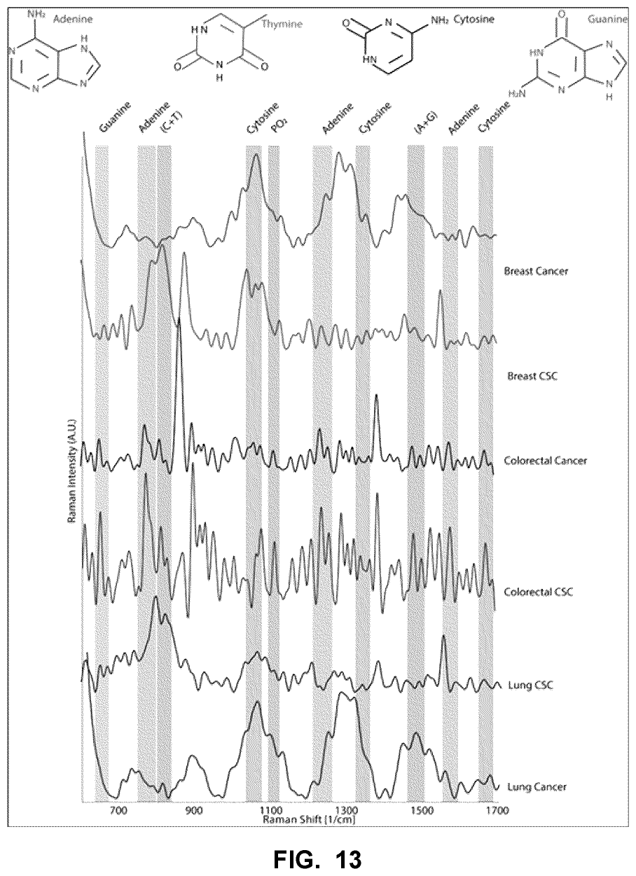

For identification of the type of cancer, Raman profiles of cancer stem cell DNA were analyzed. Existing technologies focused on analysis of genomic mutations alone have failed to identify the tissue of origin. Genomic mutation analysis alone cannot identify the underlying tissue of origin because many common gene mutations are shared amongst many cancer types. Few studies have proposed to use assays combining genetic alteration analysis with protein analysis. But such studies demonstrated very low specificity in identification of cancer location for hard to detect cancers like breast, colorectal and lung cancer. In this study, a methodology for localization of cancer has been identified by combining MCSC from cancer cell DNA along-with CSC cell DNA. As shown FIG. 13, Raman profile of cancer cell DNA and CSC DNA demonstrated significant variation. Peaks at 663 cm-1 assigned to the ring breathing modes of guanine, 733 cm-1 for ring breathing mode of adenine in DNA and RNA bases, 792 cm-1 for DNA backbone O—P—O, and ring breathing mode of cytosine and thymine, 1026 cm-1 for cytosine 1089 cm-1 assigned to PO2− stretch, 1326 cm-1 for guanine (B,Z marker) 1413 cm-1 for cytosine 1460 cm-1 assigned to the DNA and RNA bases of adenine and guanine 1555 cm-1 assigned to the ring breathing mode of adenine and 1630 cm-1 for ring breathing mode of cytosine were evident.

Breast Cancer—As per FIG. 13, breast CSC DNA showed more intensity for the peaks at 735 cm-1 assigned to DNA, 788 cm-1 assigned to the ring Breathing mode Cytosine, Uracil, 810 cm-1 assigned to O—P—O stretching of DNA, 876 cm-1 assigned to ribose vibration whereas breast cancer DNA showed higher intensity for the peaks at 600 cm-1 assigned to nucleotide conformation, 915 cm-1 for RNA, 1064 cm-1 assigned to nucleic acid, 1286 cm-1 nucleic acids and phosphates, 1315 cm-1 guanine (B-Z marker), 1560 cm-1 assigned to guanine, 1605 cm-1 assigned to cytosine. More intensity of the biomolecular peaks represented higher content of DNA components. The principal component analysis (PCA) was undertaken as PCA reduces the dimensionality of data while preserving its variability as much as possible. On plotting PC1 Vs PC2, it was observed that two clusters of cancer cell DNA and CSC DNA were formed on opposite sides of the axis. This was attributed to the negative correlation between cancer cell DNA and CSC DNA.

Colorectal Cancer—Colorectal CSC demonstrated higher intensity of the peaks at 600 cm-1 assigned to nucleotide Confirmation, 658 cm-1 assigned to G+T ring breathing and G Backbone in RNA, 670 cm-1 assigned to Ring breathing mode G, 723 cm-1 assigned to DNA, 792 cm-1 assigned to Ring Breathing mode Cytosine, Uracil, 820 cm-1 assigned to O—P—O stretching of DNA, 914 cm-1 assigned to RNA, 974 cm-1 assigned to RNA, cm-1 assigned to 1095 cm-1 assigned to Nucleic Acid, 1150 cm-1 assigned to Cytosine, Guanine and 1342 cm-1 assigned to Polynucleotide chain (DNA purine bases) whereas colorectal cancer DNA showed intense peak at 875 cm-1 assigned to Phosphodiester, Deoxyribose. PCA demonstrated distinct clustering of cancer DNA and CSC DNA.

Lung Cancer—The analysis of lung cancer cell DNA and lung CSC DNA demonstrated variation in the signature Raman bands in CSC DNA. Lung cancer DNA demonstrated higher intensity at 600 cm-1 assigned to nucleotide Confirmation, 1060 cm-1 assigned to PO2− stretch, 1252 cm-1 assigned to Guanine, Cytosine, 1282 cm-1 assigned to T,A, 1320 cm-1 assigned to Ch3CH2 wagging modes present in DNA purine bases, 1451 cm-1 assigned to Deoxyribose, 1490 cm-1 assigned to DNA whereas lung CSC showed higher intensity at 687 cm-1 assigned to ring breathing mode G, 733 cm-1 assigned to DNA, 787 cm-1 assigned to Ring Breathing mode Cytosine, Uracil, 818 cm-1 assigned to O—P—O stretching of DNA, and 1180 cm-1 assigned to Cytosine, Guanine. Higher peak intensity was attributed to more amounts of the molecules The principal component analysis (PCA) showed two clusters of cancer cell DNA and CSC DNA on opposite sides of the axis. This was attributed to the negative correlation between cancer cell DNA and CSC DNA.

Quantum superstructure assisted MCSC was instrumental in Raman profiling of DNA derived from cancer cells and CSCs demonstrated the ability of the superstructures for ultrasensitive detection. Quantum superstructures were able to obtain minute spectral differences between the DNA derived from different cells types of same cancer. This ultra-sensitive ability of the quantum superstructures was attributed to the molecular level detection capacity. CSCs are an aggressive rare subset of bulk tumor with tumor initiating capabilities. Thus, MCSC of CSC DNA to patient plasma has potential for prognosis of distant tissue invasion, tumor relapse as well as treatment monitoring.

Similarity Analysis Between DNA Derived from Cancer Cells, CSC and Tumor DNA

Cancer stem cells (CSC) is a functional state of a tumor responsible for self-renewal, proliferation and apoptosis resistance. As CSCs are at the apex of tumor hierarchy, information about similarity between the DNA of CSCs and tumor DNA will provide information on many tumor properties for prediction of therapeutic response. Due to the genetic heterogeneity in cancer, different cancer types will demonstrate variation in CSC profiles. For this purpose, MCSC of DNA derived from CSCs versus DNA derived from bulk tumor cells was undertaken. Similarly MCSC of DNA derived from non-CSC DNA versus tumor DNA was also undertaken. Analysis of the similarities associated between the tumor DNA and the DNA derived from CSC should be taken into account in development of new therapeutic approaches.

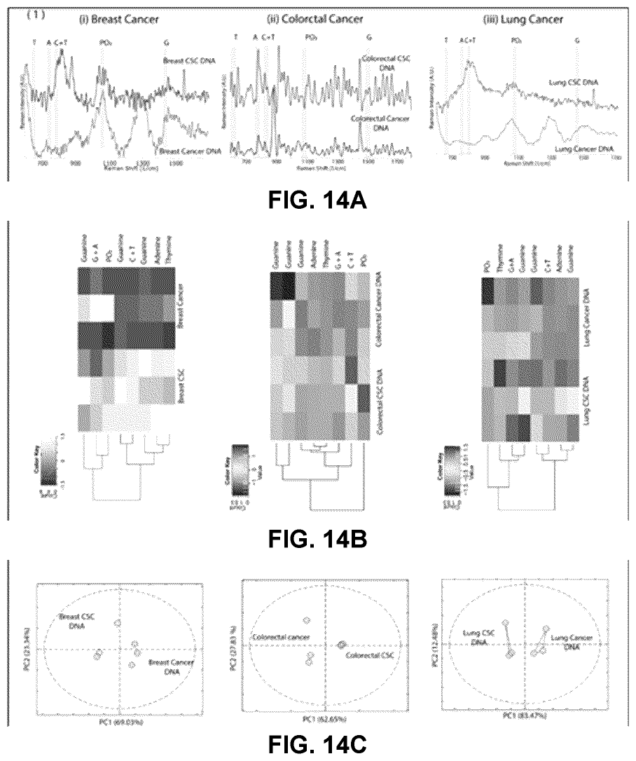

FIG. 14A shows variation in Raman profile of Cancer DNA and CSC DNA—(i) Raman spectra of breast cancer DNA and breast CSC DNA; (ii) Colorectal cancer DNA and Colorectal CSC DNA; and (iii) Lung Cancer DNA and Lung CSC DNA demonstrated variation in the peak intensity.

FIG. 14B shows a heatmap of major DNA peaks showing variation in the presence of DNA components in CSC DNA as compared to cancer cell DNA.

As shown in FIG. 14, the comparison between tumor DNA, cancer cell DNA and CSC DNA was undertaken using multivariate curve resolution (MCR) analysis. MCR is a well-established method for analysis of multi-way data especially spectroscopic data to generate its bilinear description. The loadings of MCR components demonstrated that Comp1 was comprised of mostly adenine, guanine, thymine and PO2− and O—P—O stretching of DNA. Component 2 contained Adenine, Guanine, cytosine and DNA purine bases. Component 10 was mostly comprised of all four adenine, guanine, cytosine and thymine components. Component 16 contained deoxyribose, phosphodiester bonds and nucleic acids whereas component 18 had adenine and thymine.

MCR components were used for generation of heat maps and bi-plots. The heat maps provided information on the variances associated with each MCR component. From the heat maps, it is evident that the CSC DNA demonstrated less variance as compared to cancer cell DNA versus tumor DNA. Similarly for colorectal cancer, CSC DNA showed significant similarity to tumor DNA. For lung cancer, cancer cell DNA and CSC DNA did not demonstrate any significant variation when compared to tumor DNA.

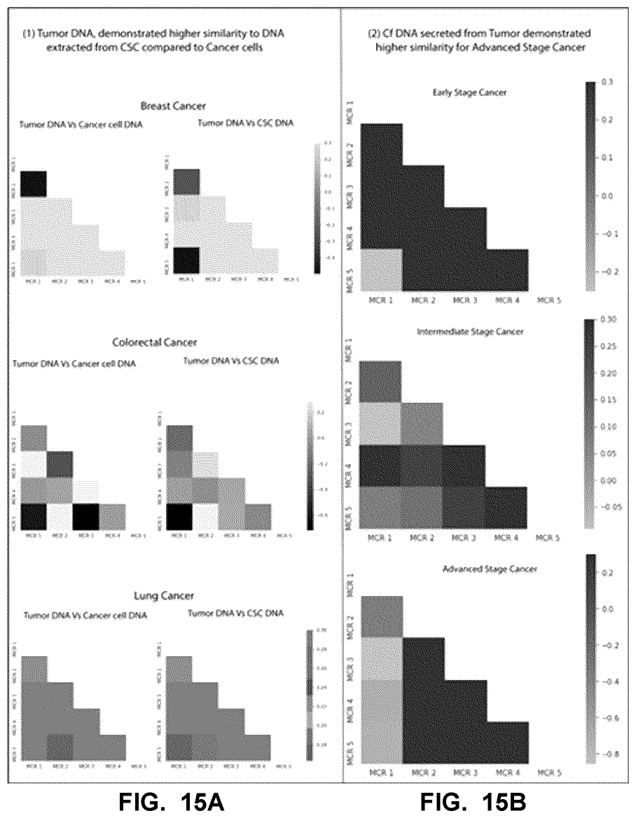

FIG. 15A shows an analysis of similarity between cancer cell DNA, CSC DNA and tumor DNA.

FIG. 15B shows a grade wise analysis that demonstrates higher correlation between tumor DNA and CSC DNA.

Similarity Analysis Between DNA Derived from CSC and Tumor DNA for Early Stage, Intermediate Stage and Advanced Stage Cancers

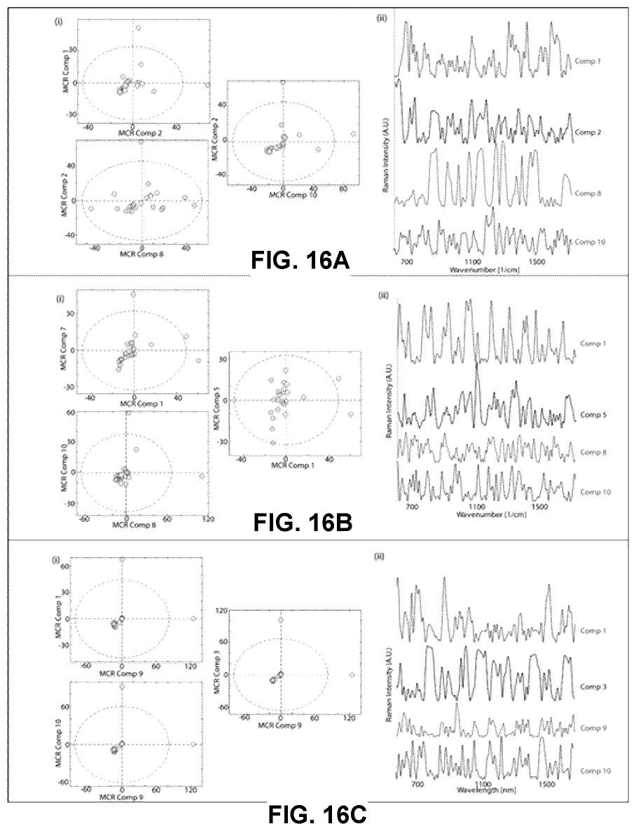

FIG. 16A shows a multiresolution curve analysis and Raman spectra demonstrating substantial similarity between cfDNA and tumorDNA for breast cancer.

FIG. 16B shows a multiresolution curve analysis and Raman spectra demonstrating substantial similarity between cfDNA and tumorDNA for breast cancer.

FIG. 16C shows a multiresolution curve analysis and Raman spectra demonstrating substantial similarity between cfDNA and tumorDNA for lung cancer.

Liquid profiling by MCSC of cancer cell DNA, CSC DNA and tumor DNA demonstrated that CSC DNA showed significantly low variance as compared to cancer cell DNA. This led us to undertake similarity analysis between CSC DNA and tumor DNA to investigate the variance from cancer development point of view. Tumor grade wise comparison was then undertaken. 10 μL of DNA derived from patient blood plasma and from patient tumor was directly dropped on the quantum superstructures and Raman spectra were captured. Pair-plot of early stage tumor DNA (grade 1), Intermediate stage tumor DNA (grade II&III) and advanced stage tumor DNA (grade IV) was undertaken using multivariate curve resolution analysis. As per FIG. 16, the early stage tumor DNA had much higher variance as compared to intermediate stage tumor DNA with CSC DNA. Advanced stage tumor DNA demonstrated lowest variance, therefore highest similarity to CSC DNA, demonstrating accumulation of more CSCs during the advanced stage cancers.

The correlation between tumor DNA and CSC DNA has potential to predict the cancer stages and therefore survival analysis for patients. As tumor stage can provide information on disease prognosis, analysis with CSC DNA is important. Presence of more CSC DNA will lead to poor prognosis, hence by detection of higher similarity of CSC DNA to tumor DNA can potentially provide accurate prognosis. Here, the potential of quantum superstructure-based MCSC for detection of biomolecular similarity analysis of CSC DNA and Tumor DNA for cancer prognosis was demonstrated.

Applicability of Patient Blood Plasma without DNA Isolation for Cancer Diagnosis

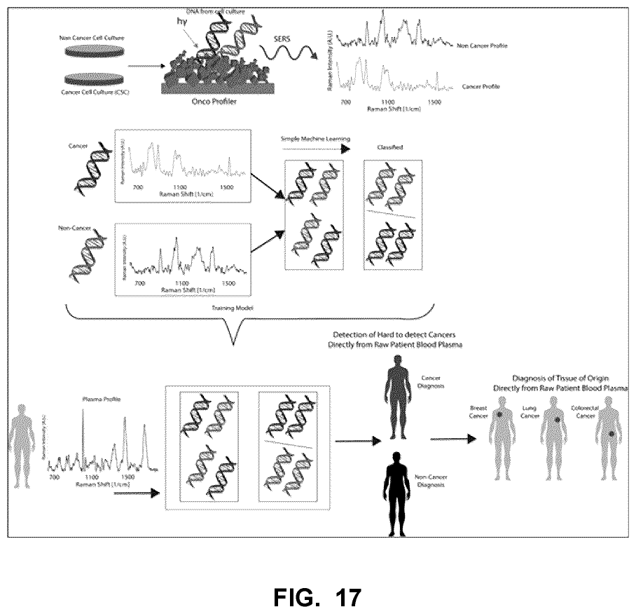

FIG. 17 shows a schematic illustration of quantum superstructure-assisted liquid profiling by MCSC of cfDNA-based diagnosis of hard to detect cancers with a simple machine learning training model.

Evaluation of the similarities between the cell free DNA (cfDNA) and tumor DNA was undertaken. This analysis was undertaken to gain insights on the concordance in the liquid profiling by MCSC of two types of bioactive substances. Quantum superstructure-based Raman profiling demonstrated substantial similarities between cfDNA and tumor DNA, enabling the use cfDNA as a detection marker. It was hypothesized that the cfDNA from plasma has the ability to demonstrate features of tumor DNA. To test the hypothesis, 10 μL of DNA solution derived from patient blood plasma and patient tumor was directly dropped on the quantum superstructures and Raman spectra was obtained. Multivariate curve resolution alternative least square (MCR-ALS) analysis was undertaken. As per FIG. 17, it was evident that the MCR component scores for cfDNA and tumor DNA clustered in one cluster and there was no demarcation or clustering amongst the DNAs showing similar properties of both DNAs. MCR-ALS decomposition of Raman spectra has the ability to provide realistic description of the pure components of the mixture.

Breast cancer MCR-AIs analysis demonstrated similar composition of MCR component scores for cfDNA and tumor DNA. Similar scores demonstrated similarity between the components. On analysis of the loadings, it was evident that comp 1 showed dominance of A+G+T whereas comp 2 had majority of the peaks assigned to T+G+C. Comp 8 mostly showed the peaks assigned to A and comp 10 had dominance of the peaks assigned to G. For colorectal cfDNA and tumor DNA, evidence of the single clustering of all cfDNA as well as tumor DNA samples confirmed similar features. The MCR loadings for comp 1 showed dominance of A+T, comp 5 had more peaks assigned to A+G, comp 8 showed mostly peaks for A whereas comp 10 had more peaks assigned to T. Lung cancer MCR components were divided into comp 1 (Adenine-A), Comp 3 (cytosine+thymine—C+T), Comp 9 (cytosine+Guanine—C+G) and comp 10 (adenine+thymine—A+T).

Covariance matrix was plotted. As shown in FIG. 17, variance between the MCR components was less than 50% for all the major components. Highest variance was observed between MCR1 and MCR10 (49%) whereas lowest variance was observed between MCR8 and MCR10 (−6%) in breast cancer. For colorectal cancer, highest variance was observed between MCR 1 and MCR 5 (41%) and lowest variance between MCR5, 8 & 10 (8%). Highest variance (39%) between MCR 1 and MCR 9 whereas lowest variance (9%) between MCR 3, 9 and 10 was observed for lung cancer. Variance of less than 50% further confirmed the similarity between cfDNA and tumorDNA. Thus, cfDNA present in plasma can be used as a biomarker for cancer diagnosis. Analysis of the MCR loadings of MCR components demonstrating lowest variance enabled us to determine that the peaks assigned to A, C, T and G can be employed as the significant peaks for blood-based diagnosis. The possibility of using plasma without isolation of cfDNA to detect presence of cancer as well as to identify the tissue of origin was then explored.

Diagnosis of Cancer and Non-Cancer Directly from Plasma

Quantum superstructure-based MCSC of DNA extracted from cancer cells (breast, lung and colorectal cancer) and its respective CSCs along-with DNA from non-cancer fibroblast (NIH3T3) cell line was used as training data. Support vector machine discriminant analysis (SVMDA) was undertaken for this purpose. SVMDA is a non-linear method which undertakes calibration and application of support vector machine classification model. Patient plasma was used for validation of the model. Classification of raw plasma samples was achieved without the need for isolation of cell free DNA with 97% sensitivity and 83% specificity. The misclassification error-proportion of cancer cases incorrectly classified as non-cancer was only 5%. F1 score is the measurement of test accuracy. Fi score provided us the percentage of true positive and true negative results. An F1 score of 0.96 was achieved for this analysis showing very high accuracy of diagnosis. The precision of the model was 0.97, which is the true positivity ratio. Mathew's correlation coefficient for the cancer and non-cancer classification with patient blood plasma was 0.732.

It was observed that the sensitivity of diagnosis improved substantially on including MCSC of both CSC DNA as well as cancer cell DNA. This combination of both DNAs enabled us to tackle the heterogeneity of cfDNA. Patient's plasma data was interpreted based on cell culture DNA. With this approach, insufficient patient data for training the SVMDA can be overcome by easily available cell culture DNA eliminating the limitation of accuracy and reliability.

Identification of Tissue of Origin Directly from Blood Plasma

Identification of tissue of origin is of crucial importance in decision making on site-specific therapy. Here, the ability of the quantum superstructure-assisted liquid profiling was tested by MCSC for identification of the tissue of origin. For undertaking tissue specific molecular Raman profiling, CSC DNA was employed in addition to tumor DNA to locate the original location of cancer. By generating a data bank of Raman profiles of various types of tumor DNA and CSC DNA, there is a potential to design a rapid testing platform which can locate the cancer with very high sensitivity and specificity.

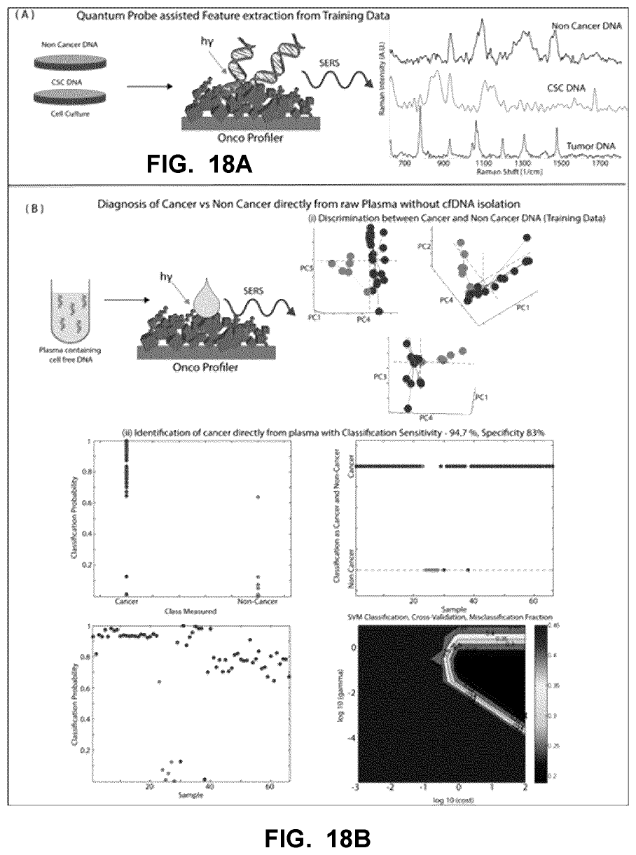

FIG. 18A shows a schematic illustration of quantum probe-assisted feature extraction from training data.

FIG. 18B shows a schematic illustration of detection of presence of cancer directly from blood plasma without cfDNA isolation, and (i) discrimination between cancer and non-cancer DNA (training data); and (ii) identification of cancer directly from plasma with classification sensitivity.

As shown in FIG. 18, DNA from cell culture was compared for identification of lung cancer. Two classes of training data were cell culture DNA of lung cancer and cell culture DNA from the remaining cancers. The model was applied to the Raman profile of patient blood plasma. Sensitivity of 83.33% and specificity of 96.15% was achieved with partial least square discriminant analysis (PLSDA). The precision of classification was 91% with F1 score of 0.87 for detection lung cancer and Mathew's correlation coefficient of 0.815. PLSDA algorithm is a versatile algorithm which can undertake predictive and descriptive modelling very effectively. Once classification of lung cancer was achieved from the mix of all patient plasma, the remaining analysis was undertaken using two types of cancers at a time. First identification of lung cancer vs breast cancer was undertaken. Breast cancer was classified from lung cancer with 86% sensitivity and 92% specificity. The precision was 92.3% and F1 score of 0.889 and Mathew's correlation coefficient of 0.772. Tumor DNA profile was added to cell culture DNA profiles for training model this time, as only cell culture DNA could not provide satisfactory results. For classification between colorectal cancer and lung cancer, sensitivity of 75% and specificity of 92% were achieved. The precision of classification was 90% with F1 score of 0.82 and Mathew's correlation coefficient of 0.68.

Quantum superstructure assisted liquid profiling by MCSC of raw patient blood plasma, achieved identification of tissue of origin of hard to detect cancers. Existing limitation of current cfDNA-based technologies to identify the tissue of origin was eliminated in this study by undertaking ultra-sensitive comprehensive profiling of the DNA structure. This was possible due to the ability of quantum superstructures to extract information on cancer-specific cfDNA directly from patient blood plasma which preserved the integrity of DNA structure resulting in very high sensitivity and specificity.

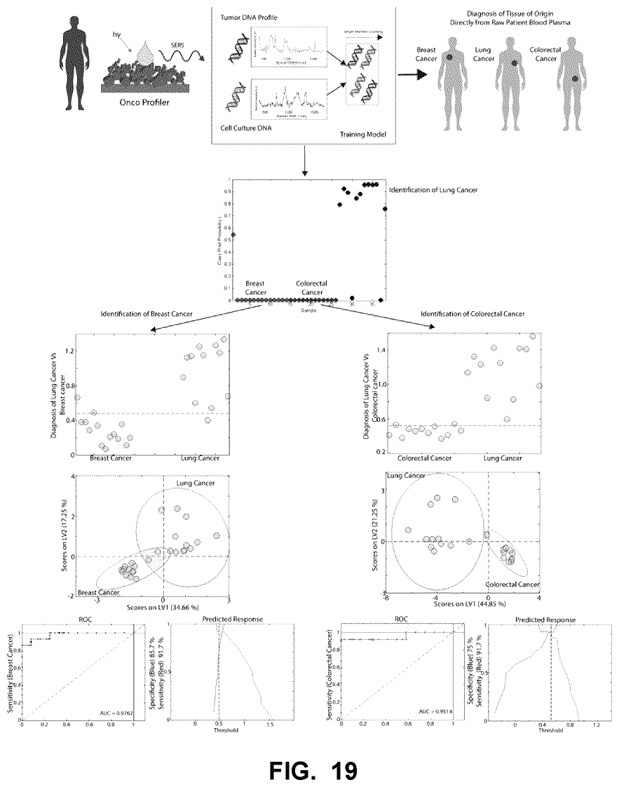

FIG. 19 shows identification of tissue of origin directly from raw blood plasm, collection of spectroscopic data from cell culture DNA of cancer stem cells and tumor DNA for identification of tissue of origin, identification of lung cancer (sensitivity 83% specificity 96%), breast cancer (sensitivity 86% specificity 92%) and colorectal cancer (sensitivity 75% specificity 92%).

Early Detection of Brain Cancer with Exosomes in Patient Serum

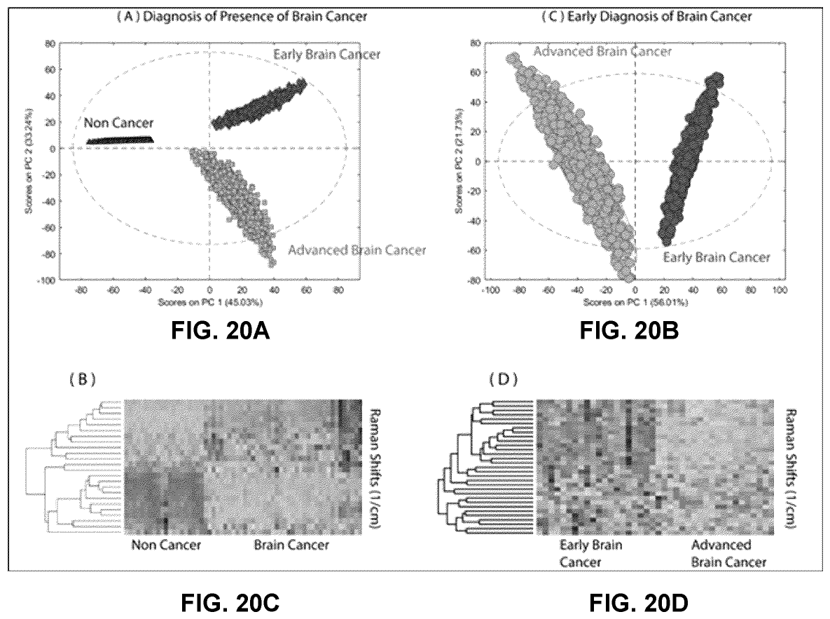

FIG. 20A shows principal component analysis demonstrating clear clustering between non cancer EVs vs early and late cancer EVs.

FIG. 20B shows a heatmap of the loadings of PC1 demonstrating the ability of our methodology to discriminate between brain cancer and non-cancer.

FIG. 20C shows a PCA demonstrating clear clustering between early brain cancer vs advanced brain cancer.

FIG. 20D shows a heatmap of the loadings of PC2 demonstrating the ability of the methodology to discriminate between early grade brain cancer vs advanced brain cancer.

Ability of the Nanosensor to Predict the Malignancy of Brain Tumor with a Small Amount of Patient Serum

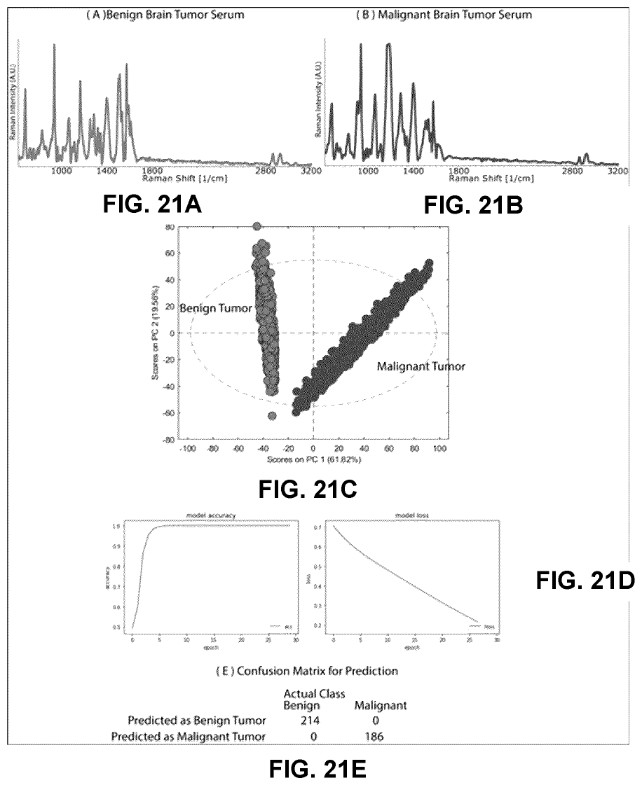

FIG. 21A shows a Raman spectra of serum of a patient with a benign brain tumor.

FIG. 21B shows a Raman spectra of serum of a patient with a malignant brain tumor.

FIG. 21C shows distinct clustering of tumor types demonstrating ability of the methodology for clear discrimination

FIG. 21D shows ANN demonstrated 100% accuracy for prediction of the malignancy of tumor

FIG. 21E shows a confusion matrix for malignancy prediction.

Preliminary Results for Methylation of ctDNA

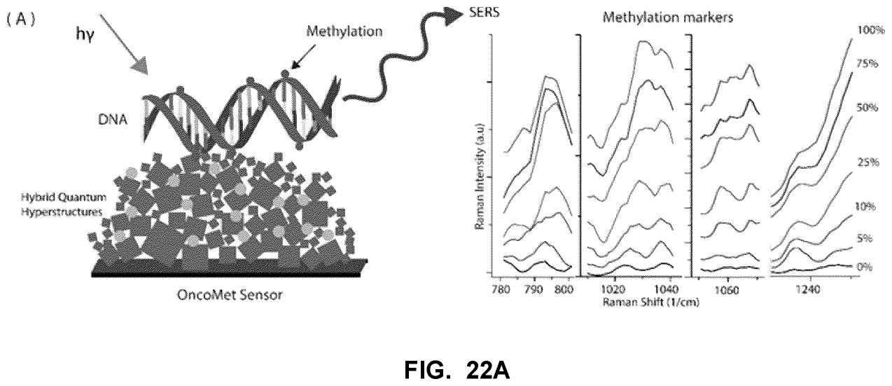

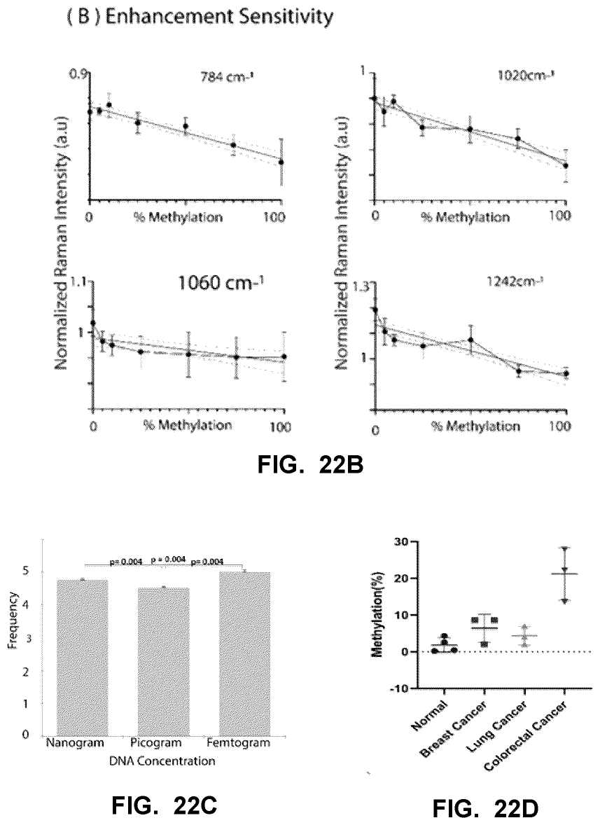

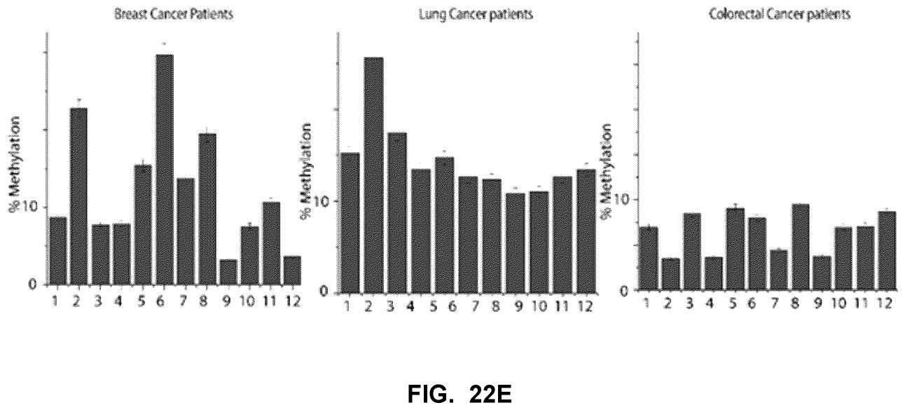

Turning now to FIGS. 22A-E, shown therein are images for assessing the SERS activity of a sensor as described herein using DNA as a Raman active molecule. Specifically, FIG. 22A shows a schematic representation of the sensor with hybrid quantum hyperstructures generating preferential adsorption of methyl groups and a SERS spectra of DNA with differential methylation percentage. FIG. 22B shows SERS peak intensity analysis to showcase the enhancement efficiency of the sensor. FIG. 22C shows ultra-high limit of detection (Femto gram DNA concentration). FIG. 22D shows quantification of methylation from preclinical models. FIG. 22E shows quantification of circulating methylation.

DNA methylation is a crucial diagnostic marker for cancer metastasis. Studies have shown that methylation levels in DNA are positively correlated with cancer metastatic potential and are highly tissue specific. Here, the quantum hyperstructures for the detection of methylation levels in DNA have been applied.

FIG. 22A shows the baseline-corrected SERS spectra of DNA with precalibrated differential methylation. The most informative spectral regions for DNA methylation are 1) 700 cm⁻¹-800 cm⁻¹ corresponding to ring breathing vibrations of cytosine, in particular, the peak at 784 cm⁻¹ where the peak intensities show a decreasing trend with increasing methylation percentage 2) 950 cm⁻¹-1150 cm⁻¹ corresponding to the intense stretching vibration of phosphodioxy molecule. The spectra also show the characteristic peaks associated with DNA. Each of the methylation markers is normalized with the PO₂ peak at 1089 cm⁻¹. The peak corresponding to PO₂ remains stable irrespective of the concentration and molecular modifications of DNA.

Further, the presence of the PO₂ peak also provides information on any structural damage to DNA. Based on the SERS spectral data, the spectral features which vary the most with varying methylation percentage were monitored. It was concluded from FIGS. 22A and 22B that the SERS peaks at 784 cm⁻¹, 1020 cm⁻¹, 1060 cm⁻¹, 1242 cm⁻¹ are best suited for quantification of global methylation levels in DNA. Additionally, it can be observed from FIG. 22B that the intensity of methylation markers increases with an increase in methylation percentage.

Next, the quantum hyperstructures were applied to quantify DNA global methylation. Here, a pre-calibrated DNA was used with differential methylation percentage to establish a calibration curve between SERS intensity and methylation percentage (FIG. 22B). By applying the methylation markers ascertained, a linear relationship exists between SERS intensity of the methylation markers and the methylation percentage.

Principal component analysis was performed to differentiate between the different methylated samples. From the established PCA model, the seven different methylated groups are distinguishable from each other with high specificity and sensitivity. Quantitative analysis of DNA methylation is essential for the diagnosis of cancer metastasis. There exists a linear correlation between the normalized SERS intensity of the DNA methylation markers and methylation %. The correlation equation was calculated as Y=−0.002050*X+0.7661 with an R² value of 0.7025. The correlation equation is further used to quantify DNA methylation in genomic DNA isolated from preclinical cancer models. These results showcase a quantitative correlation between global DNA methylation levels and the spectral features identified as methylation markers in this study.

Studies have shown that DNA methylation patterns in the primary tumor contains critical information on the metastatic potential, hence can be utilized to determine cancer progression. It can be observed from FIG. 22 that the sensor described herein requires only minimal input DNA for detecting cancer progression.

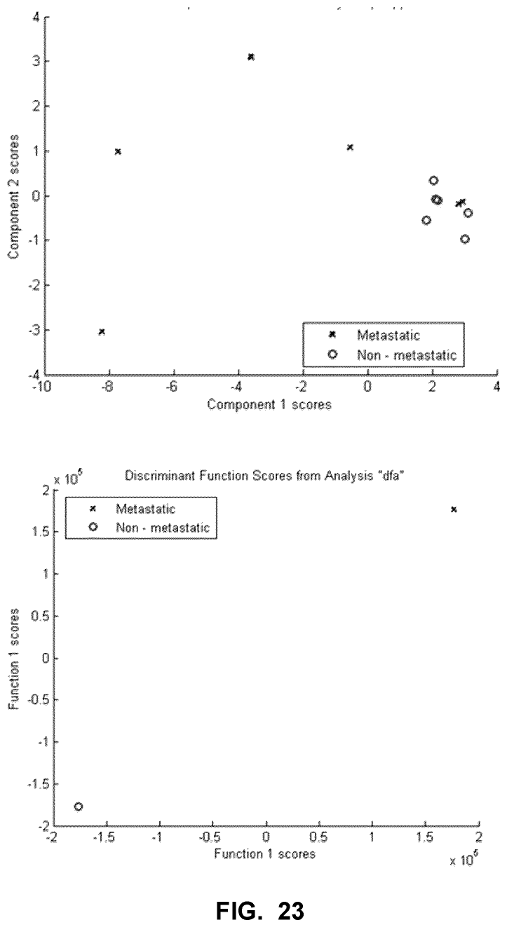

FIG. 23 shows two graphs that provide discrimination of circulating free DNA (cfDNA) methylation signature between metastatic and non-metastatic tumor samples showcasing the ability to diagnose metastatic potential of methylation in cfDNA.

### Preliminary Results for Exosome Based Cancer Metastasis Detection

It should be noted that colorectal and breast cancer cells are metastatic whereas lung cancer cells are non-metastatic. The content of metastatic and non-metastatic proteins shows variation in the content of the extracellular vesicles of each type of cancer and its CSC counterpart. This variation is cancer type specific. By in-depth analysis of these variations, detection of metastasis can be undertaken.

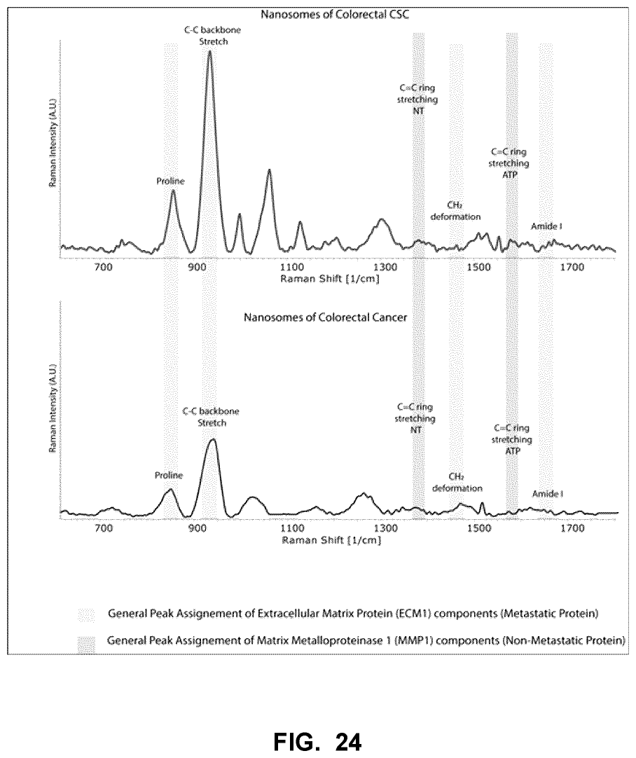

FIG. 24 shows a Raman spectral comparison of extracellular vesicles derived from colorectal cancer cells and colorectal CSCs to determine the signature of metastatic and non-metastatic proteins in nanosomal cargo.

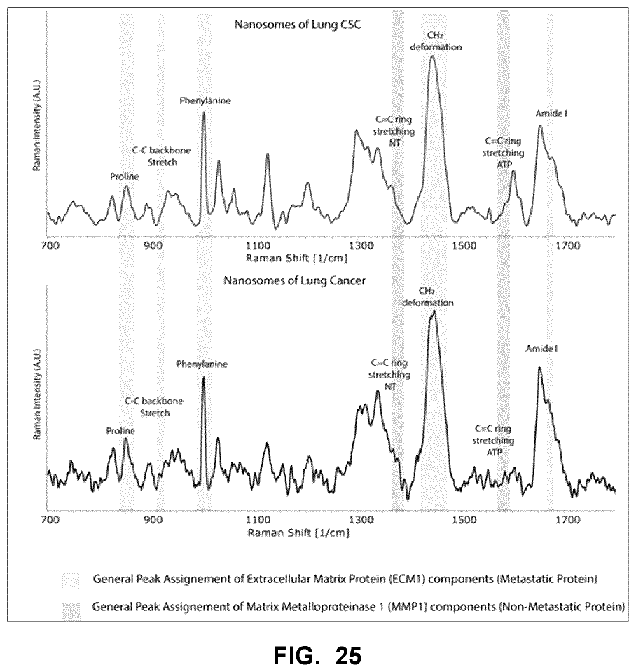

FIG. 25 shows a Raman spectral comparison of extracellular vesicles derived from lung cancer cells and lung CSC to determine the signature of metastatic and non-metastatic proteins in nanosomal cargo.

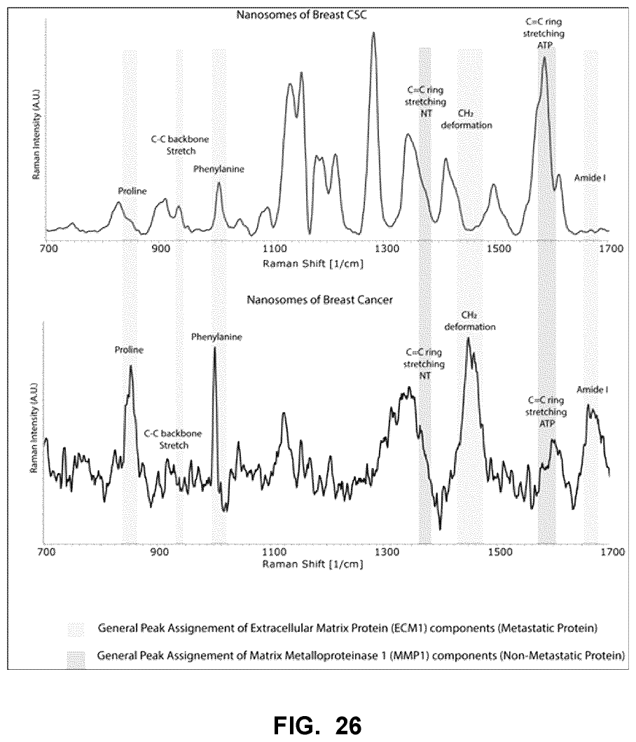

FIG. 26 shows a Raman spectral comparison of extracellular vesicles derived from breast cancer cells and breast CSCs to determine the signature of metastatic and non-metastatic proteins in nanosomal cargo.

### Sensor-Assisted Global DNA Methylation Signal Enhancement Mechanism

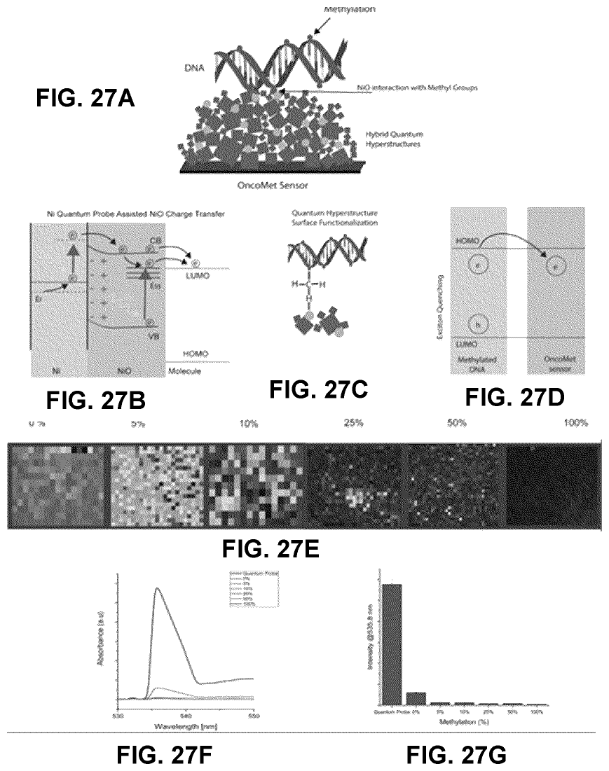

FIG. 27A shows a schematic representation of DNA adsorbed on the Quantum Hyperstructures.

FIG. 27B shows presence of metal assisted semiconductor charge transfer resonance.

FIG. 27C shows surface functionalization of quantum hyperstructures for selective adsorption of methyl groups.

FIG. 27D shows exciton quenching resulting in substantially enhanced SERS.

FIG. 27E shows charge transfer resonance showcased by the presence of photoluminescence quenching with increasing methylation percentage confirming the effective trapping of methylated DNA.

FIG. 27F shows absorption spectra to showcase the reduction in fluorescent intensity.

The 3D architecture arrangement of quantum hyperstructures comprises metallic Ni (cube) decorated with semiconductor NiO (spherical), which contributes to multiple SERS enhancement mechanisms. The primary enhancement arises from the electromagnetic enhancement due to the cube-shaped metallic Nickel's sharp corners. FIG. 27A shows the presence of sharp corners in the cubical Ni sensors. The presence of sharp corners enables effective photon confinement, thereby providing a high amplification of signals. Engineering hotspots through morphological modification is critical to acquire strong electromagnetic enhancements, which was achieved using the cube shaped Ni. Traditionally plasmonic/electromagnetic enhancement is criticized due to the lack of reproducible signal.

In addition to engineering hotspots through morphological modifications, there is also a need for a secondary boosting mechanism, which is uniform, reproducible, which is provided by the NiO on the surface of the metallic Nickel. Here, the presence of semiconductor NiO enables effective charge transfer by facilitating adsorption between the methylated DNA and NiO surface. The XPS analysis revealed the presence of —OH functional groups on the surface of the sensor, which interacts with the methylated DNA, thereby enabling methylation specific adsorption. Further, the quantum size of the probe provides a high surface area for adsorption.

Modulating the relationship between SERS and photoluminescence is critically important for enhancing SERS signals. The quantum hyperstructures specifically trap the methylated DNA bases through molecular adsorption. The molecular adsorption enables charge transfer between DNA molecule and the quantum hyperstructures system. It can be observed from FIG. 27C, that the charge transfer leads to photoluminescence quenching, which also explains the methylation-specific enhancement of SERS signals. Additionally, the absorption spectra shown in FIG. 27E confirms the photoluminescence quenching with increasing percentage of methylated DNA bases.

Collectively, the quantum hyperstructures amplifies the methylation-specific signatures through multiple mechanisms: i) plasmonic enhancement due to the morphological modifications of Ni ii) Molecular adsorption because of the surface functional groups on the NiO in combination with the effective quenching of excitons enables a 1000-fold amplification of methylation related signals.

### Quantum Hyperstructures of Sensor for Mapping Circulating Methylation for Metastasis (MCMM)

A tumor comprises a highly heterogeneous population of cells. The intratumoral heterogeneity is contributed by the small subpopulation of cells known as cancer stem cells (CSCs). CSCs are shown to undergo asymmetric cell division, further increasing the heterogeneity of a tumor and leading to tumor metastasis. Further, CSCs have also been shown to possess high levels of global hypermethylation compared to non-CSCs. Recent studies have shown that epigenetic changes are critical events in cancer development and epigenetic changes in cancer cells increase the predisposition to metastatic transformation and formation of CSCs. Additionally, DNA methylation patterns in CSCs revealed the dynamics of CSC expansion dynamics and the mechanism of tumor evolution in colorectal cancer. Hence, to design a methylation-based diagnostic test for cancer metastasis with high accuracy and specificity, it is essential to consider methylation patterns of both CSCs and cancer cells since it presents a holistic representation of a tumor.

Aberrant DNA methylation in CSCs plays a primary role in cancer progression, cancer growth and shapes the intratumor heterogeneity. Studies have proven that DNA methylation in CSCs is significantly hypermethylated when compared to bulk tumor cells. The DNA of cancer cells and CSCs derived from preclinical models of breast cancer, lung cancer, and colorectal cancer were compared. Correlation analysis shows the clustering of CSC and cancer DNA in distinctly different clusters without overlap. The correlation analysis between the global methylation levels of CSCs and cancer shows a similar negative correlation across different cancers.

Further, the correlation heatmap (FIGS. 27A and 27B) also confirm the negative correlation between CSC DNA methylation status and Cancer DNA methylation status. PCA analysis is shown in FIGS. 27A and 27B also confirms the distinct methylation patterns between CSC DNA and cancer DNA across different tissues. A similar correlation across different tissue types confirms the global methylation status of DNA to be applied as a marker for tumor tissue of origin diagnosis.

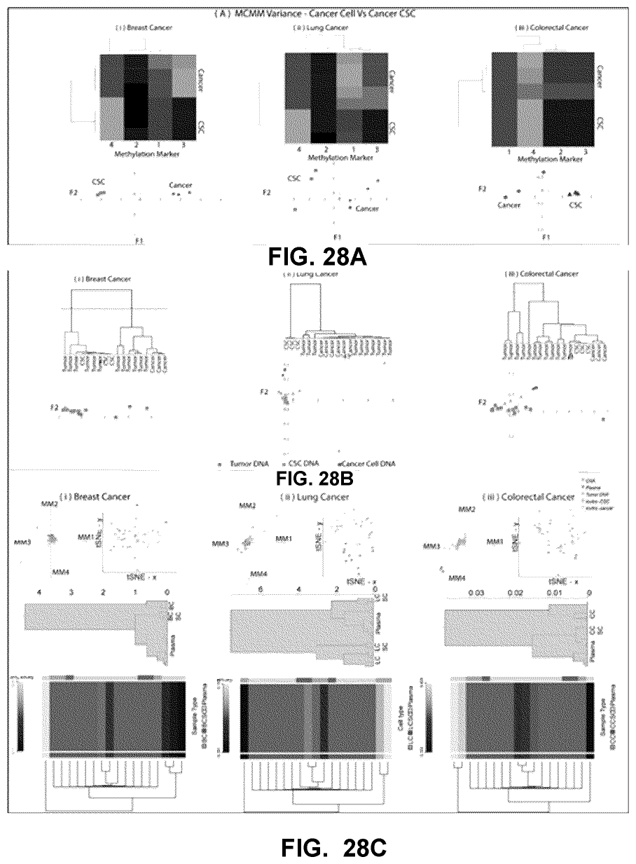

FIG. 28A shows Mapping Circulating Methylation for Metastasis (MCMM) instrumental in analysis of variance associated with global methylation of cancer cells vs cancer stem cells (CSC).

FIG. 28B shows results for cancer cell, CSC and tumor DNA being analyzed for investigation of associated similarities with MCMM

FIG. 28C shows results of cancer cell, CSC, Tumor DNA and plasma being investigated to analyze associated similarity for (i) Breast cancer (ii) Lung Cancer and (iii) colorectal cancer.

Next the DNA methylation markers of cancer and CSC DNA with DNA isolated from tumor biopsy was compared to establish the similarities between DNA methylation patterns in CSC and cancer cells in preclinical models and DNA from tissue biopsy. The DNA from tumor biopsy (Tumor DNA) includes the DNA features representing intratumor heterogeneity. The DNA methylation markers in preclinical models can be extended for clinical application if there is a highly significant similarity between DNA from preclinical models and tumor biopsy DNA. Here, a Euclidean distance analysis was applied to showcase the similarity between in vitro DNA and tumor biopsy DNA (FIG. 28C) that both tumor DNA and in-vitro DNA (cancer+CSC) fall under two classes with 87.5% similarity within classes and 12.5% similarity between classes.

Further, the distribution of tumor DNA in both classes is correlated to the presence of CSCs. A similar trend is observed in colorectal cancer, where in-class similarity was observed as high as 92.7%, whereas between class similarity of 7.28%. However, in lung cancer, the CSCs form a separate class, and the tumor DNA and the cancer cell DNA fall under the same class with a 93.73% similarity. Further, the lung CSC show only 6.27% similarity to tumor DNA. This low similarity could be attributed to the predominance of primary non-metastatic lung cancer.

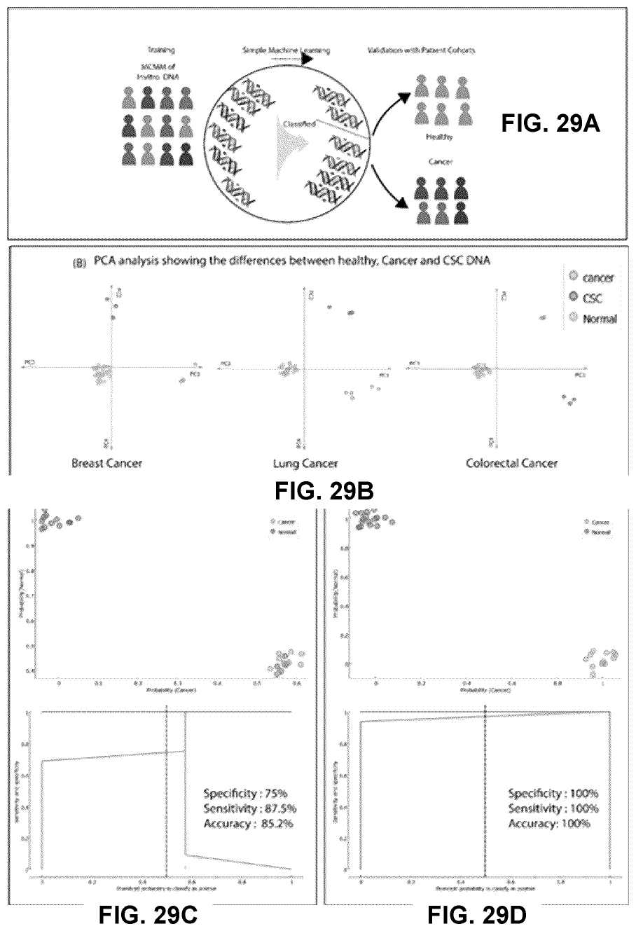

FIG. 29A shows a schematic illustration for the application of MCMM for cancer diagnosis.

FIG. 29B shows a PCA analysis showing the differences between healthy, cancer and CSC DNA instrumental in cancer diagnosis.

FIG. 29C shows MCMM for cancer diagnosis using cancer DNA shows 85% accuracy.

FIG. 29D shows MCMM for cancer diagnosis using CSC DNA shows 100% accuracy, validating the applicability of CSC methylation as a viable marker for cancer diagnosis.

Additionally, PCA analysis showcased in FIG. 29B also confirmed the overlap/similarity with tumor DNA. Based on the results, it can be inferred that combining CSC DNA and cancer DNA from preclinical models shows a significantly similar profile to the tumor DNA profile, confirming that CSC contributes to a high percentage of intratumor heterogeneity. Further, including the CSC component in the DNA can improve the diagnostic efficacy and the specificity of the metastatic profiling of cancer.

Next, the possibility of profiling DNA methylation was investigated directly from plasma. Correlation analysis was performed on the methylation markers in plasma. It can be observed in FIG. 28C that the methylation markers from plasma significantly overlap with the methylation markers of DNA from preclinical models, tumor DNA, cell-free DNA isolated from plasma. Hence, it can be confirmed that the DNA methylation markers obtained using the sensor can be applied for profiling cancer metastasis directly from plasma, thereby eliminating the complex isolation process.

A t-SNE (t-distributed stochastic neighborhood embedding) analysis was performed to investigate the similarity between the methylation markers from the subsets of samples. Like PCA analysis, the tSNE algorithm performs a dimensionality reduction to visualize the data in a 3-dimensional space. It can be observed from FIGS. 29B-D that the methylation markers from different sample subsets fall under the same cluster, confirming the application of using plasma as an analyte for cancer metastasis profiling.

DNA methylation is a critical epigenetic mechanism involved in regulating tumor-specific gene regulation such as gene silencing, transcriptional activation. Additionally, when compared to ctDNA mutations, rearrangements, the methylation status of ctDNA has multiple advantages to be applied for cancer diagnosis. First, ctDNA methylation is detectable at early stages of carcinogenesis, second the methylation pattern is highly conserved and helps in determining the tissue of origin of the malignancy.

Here, a global DNA methylation of circulating tumor DNA was applied for cancer diagnosis. It can be observed from PCA analysis in FIG. 29B, that the methylation patterns of cancer DNA, CSC DNA and DNA from normal epithelial cells form distinct clusters without overlapping across different tissue types like breast cancer, lung cancer and colorectal cancer. Hence, it can be concluded that the methylation patterns of DNA is highly conserved and can be applied to detect cancer. Further, it can be observed from FIG. 29B, that cancer DNA and CSC DNA are distinctly different.

Next, a random forest classifier was applied for cancer diagnosis using both in vitro cancer DNA and CSC DNA for training. The trained classifier was then applied to an plasma samples from an independent cohort of cancer patients for validation. First, only in vitro cancer DNA and DNA from normal epithelial cells were employed for training the classifier. The validation with plasma samples using the cancer DNA trained classifier yielded an accuracy of 85.2% (FIG. 29C). Additionally, the specificity and sensitivity was observed to be at 75% and 87.5% respectively. Next, the random forest classifier was trained with CSC DNA and DNA from normal epithelial cells. The validation with plasma samples yielded an 100% diagnostic accuracy. The specificity and sensitivity was also maintained at 100%. This confirms that the methylation patterns of CSC DNA better represents the tumor heterogeneity, hence can be applied as a reliable marker for cancer diagnosis.

Studies have shown that global DNA methylation patterns are highly tissue-specific, hence can serve as a marker for diagnosis of cancer metastasis. Based on the quantitative correlation between SERS methylation markers and methylation percentage of DNA and the high sensitivity of the sensor, the sensor was applied to distinguish cancer tissue of origin in preclinical models of metastatic and primary cancer. The fundamental diagnostic component of cancer metastasis is to determine the tissue of origin. Additionally, determining the cancer origin combined with site-specific targeting of cancer metastasis is essential to improve treatment outcomes. Studies have shown that DNA methylation is highly tissue-specific, hence can be applied to determine cancer tissue of origin and cancer localization. Further, the tissue-specific methylation patterns are conserved, even though there is cancer-related epigenetic alterations. Here, the tissue-specific methylation and cancer-related epigenetic alterations were combined to classify tumor tissue of origin.

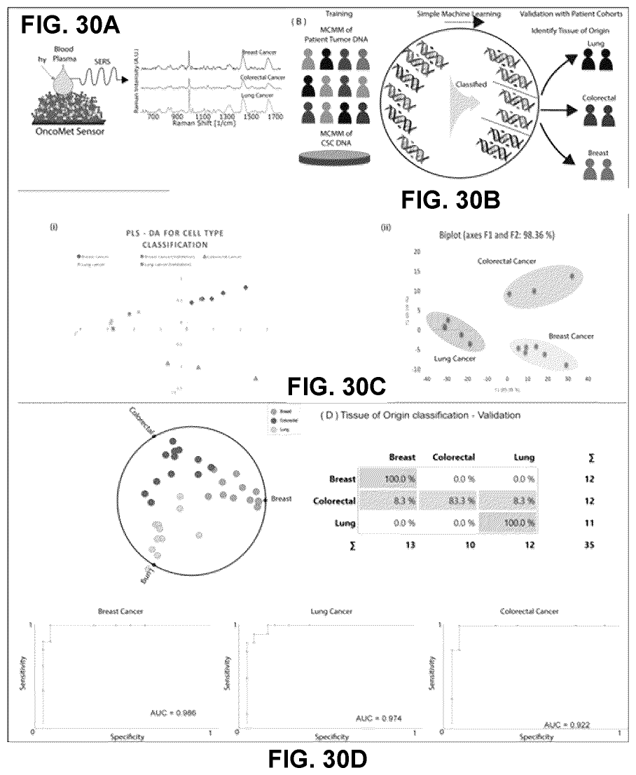

FIG. 30A shows a schematic diagram of how a sensor showed signature profiles of multiple cancer types.

FIG. 30B shows a schematic representation of the detection methodology.

The genomic DNA isolated from preclinical cancer models for metastatic colorectal cancer (COLO-205), Metastatic breast cancer (MDA-MB-231), Non-metastatic lung cancer (H69-AR) were used to determine if the DNA methylation markers can be applied to determine the metastatic status of cancer. The PCA analysis clearly distinguishes cancerous DNA from different tissue of origin (FIG. 30). In addition, PLS-DA analysis also shows the apparent variance between different cancer types (FIG. 30). It can also be observed that there is no overlap between colorectal cancer and breast cancer, even though both are metastatic.

A random forest algorithm was applied to resolve the tissue of origin of the cancer samples—the DNA methylation markers from in vitro cancer DNA, CSC DNA, tumor DNA. The trained model was then validated by applying it to plasma. It can be observed that the classification accuracy was 100% and the ROC curve generated determined the specificity and sensitivity to be 100% for preclinical models of cancer. However, when the CSC methylation markers were added, the classification accuracy was reduced to 83.9%. However, the specificity of classification was maintained at over 90%. The specificity for Lung cancer, Colorectal cancer, Breast cancer was 93%, 90%, 89%, respectively. The patient cohort's reduction in accuracy is predominant due to the cancer progression, and the cfDNA contains information on cancer progression tissue. The direct detection of tissue of origin from plasma yielded a classification accuracy of 84.4%. The specificity for Lung cancer, Colorectal cancer, Breast cancer was 95.5%, 90%, 95%, respectively.

Determining the Cancer tissue of origin is critical to decide the clinical course of action. Although studies on early detection of cancer's tissue of origin using a liquid biopsy-based approach has shown a high sensitivity, there exists a fundamental variation between the methylation changes between cancer tissue DNA and DNA in circulation. This variation could be attributed to the intrinsic differences between the tissue and plasma methylation. Hence, approaching cancer tissue of origin using a panel of methylated gene loci requires a large-scale patient cohort for validation of the diagnosis method. This study utilizes the global methylation patterns which represent the fundamental epigenetic modifications that is specific to the tissue of origin and tissue of progression.

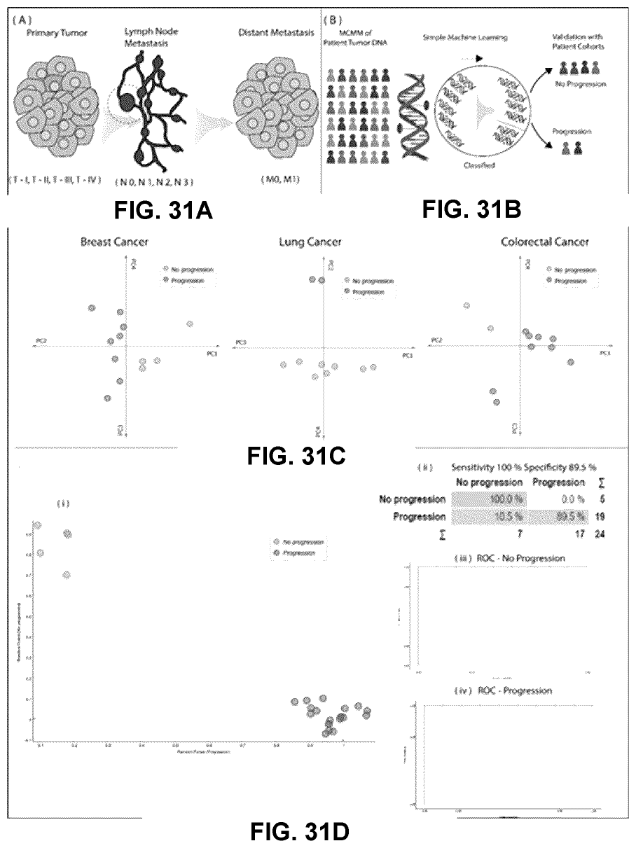

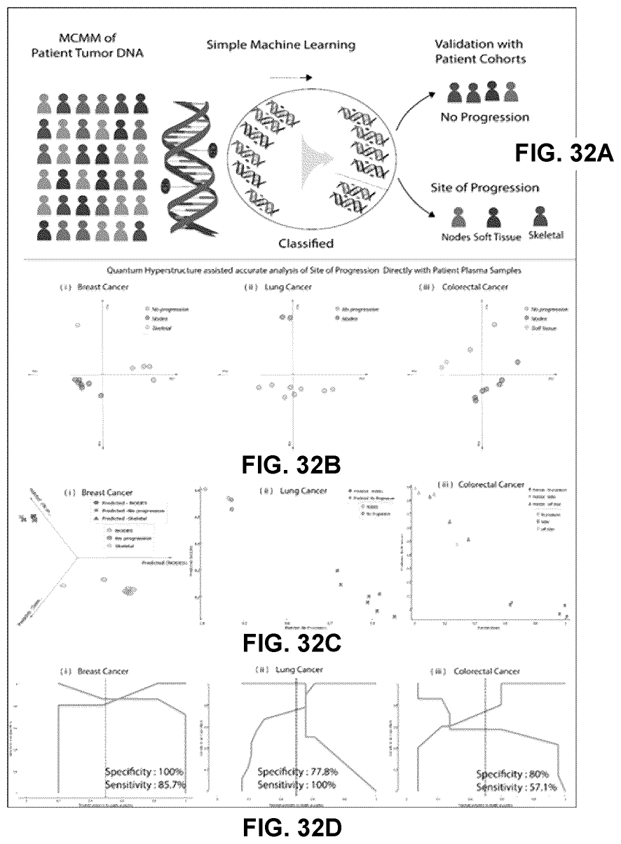

DNA methylation plays a predominant role in determining the progression status of cancer. The first step to cancer metastasis is progression of tumor cells to multiple sites such as nodes, soft tissue, bones (FIG. 31A). Hence, to monitor and perform real-time surveillance of cancer metastasis, it is essential to determine the progression status of the tumor. The PCA analysis of methylation markers shows that the methylation signatures of tumors, which are progression and No progression, show a clear distinction in colorectal cancer, lung cancer and breast cancer samples (FIG. 31C). The Random Forest classifier was trained using tumor DNA isolated from clinical tumor samples and the cfDNA isolated from the plasma samples (FIG. 31B). The trained model was applied directly to plasma to determine cancer's progression status. The Random forest model has applied to pan-cancer classification, which yielded a classification accuracy of 100% for the training data and 91.7% for the validation dataset (FIG. 31D). The ROC curve was used to determine the performance of the classification of the model. The AUC was found to be 1.00, which confirms the high performance and accuracy of the classification algorithm (FIG. 31D). The specificity for classification was found to be 100%, and sensitivity was found to be 89.5%. Further, it can also be observed from the random forest classifier plot presented in FIG. 31D that there is a clear distinction between samples with no progression and samples from cancer progressed to multiple sites without any overlap (see FIG. 32).

FIG. 32A shows MCMM of tumor DNA was used as training data.

FIG. 32B shows cancer site of progression was diagnose with multiple types of cancers: (i) Breast Cancer (ii) Lung Cancer (iii) Colorectal Cancer.

FIG. 32C shows a random forest classifier demonstrated (i) distinct clustering.

FIG. 32D shows performance of random forest classifier demonstrating high sensitivity and specificity for detection of cancer site of progression.

Diagnosis of Nodal Progression and Clinical Metastasis with Methylation in the Cell Free DNA of Cancer Stem Cell

### Detection of Nodal Metastasis

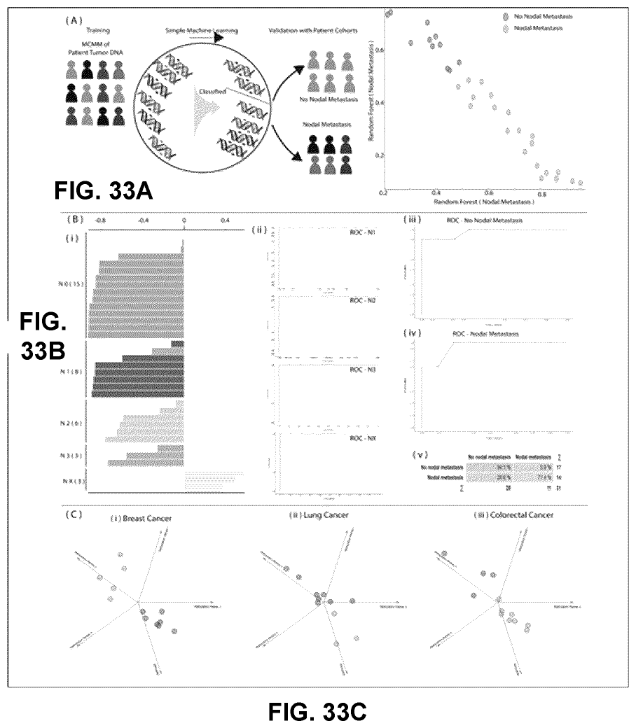

The methylation markers were applied to determine the stage of nodal metastasis. K-means clustering analysis was performed to determine the ability of different nodal grades to be classified using methylation markers. The silhouette plot in FIG. 33B shows that each grade falls into a separate cluster. Further, it can also be observed from the Gradient boosting classifier plot presented in FIG. 33A that there is a clear distinction between samples with different nodal metastatic grades. Additionally, the grade NX significantly overlaps with grade N3. Clinically, a patient is given grade NX when the conventional techniques cannot provide a grade for the tumor. The Gradient boosting classifier was trained using tumor DNA isolated from clinical tumor samples and the cfDNA isolated from the plasma samples. The trained model was applied directly to plasma to determine nodal metastasis status. The trained model has applied to pan-cancer classification, which yielded a classification accuracy of 100% for the training data and validation dataset (FIG. 33A-B). The area under the curve for ROC curve was used to determine the performance of the model's classification was found to be 1.000 for the training data and the validation dataset (FIG. 33A-B). The specificity for classification was found to be 100% across cancers.

### Detection of Clinical Metastasis

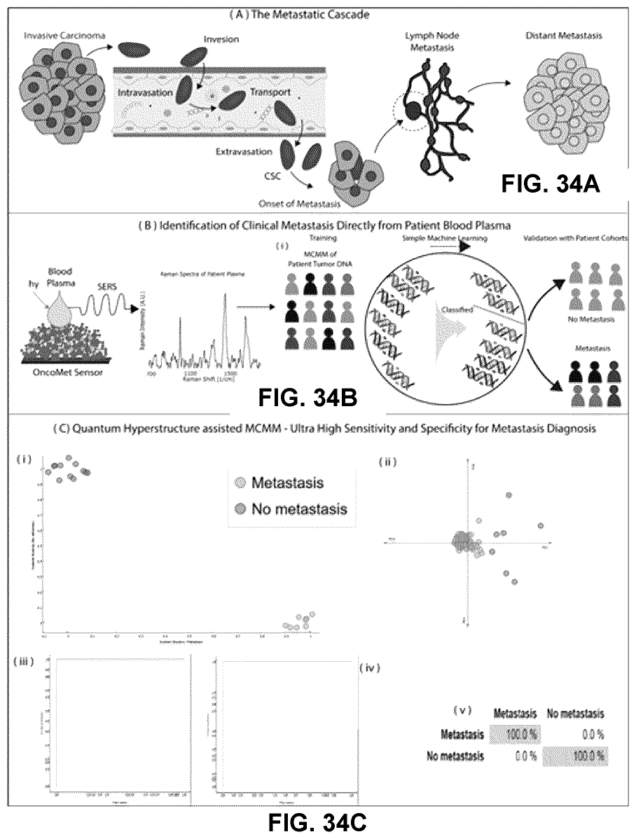

FIG. 34A shows a schematic representation of metastatic cascade.

FIG. 34B shows a schematic representation of detection of clinical metastasis directly from patient blood plasma.

FIG. 34C shows detection of metastasis was achieved with 100% sensitivity and 100% specificity.

Hypermethylation is one of the main epigenetic modifications that drive cancer metastasis. Several studies have shown the correlation between metastasis and hypermethylation across different cancer types. Since the methylation levels in a tumor are positively correlated with the methylation levels in cfDNA, the methylation markers in plasma were utilized to classify a tumor's potential to metastasize directly. PCA analysis shown in FIG. 34 shows a clear clustering in non-metastatic samples. However, the metastatic samples are highly spread but do not overlap with the non-metastatic samples. Next, a Gradient boosting classifier was applied to showcase the ability to differentiate between metastatic and non-metastatic samples. It can also be observed from the Gradient boosting classifier plot presented in FIG. 34 that there is a clear distinction between metastatic and non-metastatic samples. The Gradient boosting classifier was trained using tumor DNA isolated from clinical tumor samples and the cfDNA isolated from the plasma samples. The trained model was applied directly to plasma to determine nodal metastasis status. The trained model has applied to pan-cancer classification, which yielded a classification accuracy of 100% for the training data and validation dataset (FIG. 34). The area under the curve for ROC curve was used to determine the performance of the model's classification was found to be 1.000 for the training data and the validation dataset. The specificity for classification was found to be 100% across cancers.

### Glioblastoma Stem Cell Derived Extracellular Vesicles for Non-Invasive GBM Liquid Biopsy

Molecular Level Detection with Superlattice Sensor

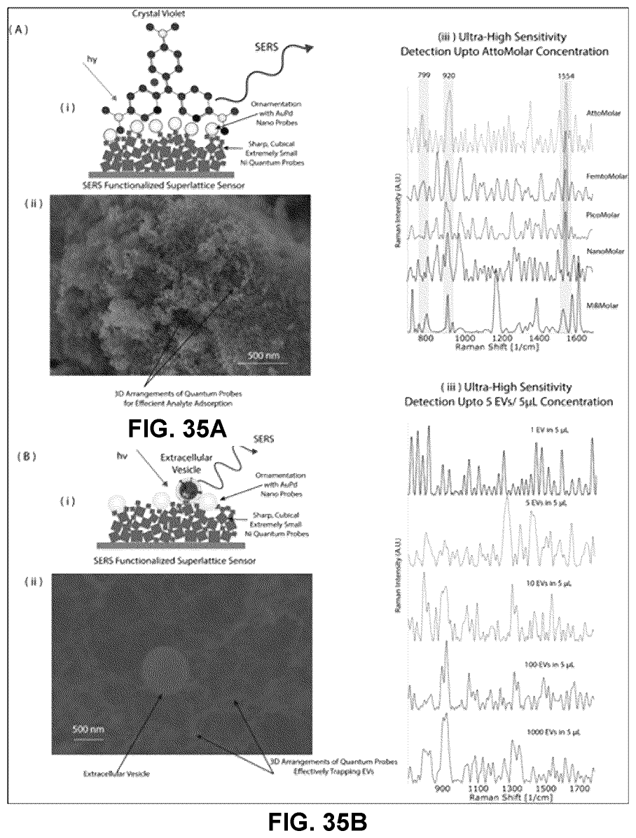

The SERS spectra of Crystal Violet (CV) molecules at different concentrations were analyzed to find out the limit of detection and thus the sensitivity of the superlattice sensor. Spectra was acquired by sequentially lowering the concentrations of CV from milliMolar to attoMolar. For this, 10 μL of CV solution was dropped on the superlattice surface and spectra was acquired using a 785 nm Raman laser. At millimolar concentration all the Raman peaks characteristic of CV molecules of Crystal Violet—Peak Assignment were observed. At lower concentrations, it can be observed from FIG. 35A(iii) that peaks at 799 cm⁻¹, 920 cm⁻¹ and 1554 cm⁻¹, corresponding to out-of-plane ring C—H bending, ring skeletal vibration and ring C—C stretching respectively, were selectively enhanced. The signal to noise ratio at attomolar concentration was calculated to be 13.04. The attained Limit of detection was 10⁻¹⁸ M for CV. This lowest LoD of attoMolar concentration achieved can be ascribed to the uniqueness of superlattice structure. The porous nature created by the Ni quantum layered cubical network facilitates enhanced adsorption of analyte molecules on the superlattice surface. The 3D layered structure of the superlattice evident from HRSEM image as shown in FIG. 35A(ii) allows penetration of CV molecules to subsequent layers rather than the surface. Besides Au/Pd nanospheroids generate LSPR due to abundant hotspots formed by closely spaced nano-quantum structures in the superlattice leading to high signal enhancement.

At attoMolar concentration, a small shift was observed for the major Raman peaks which indicates that the spectrum was possibly from a single CV molecule. Since not more than a single CV molecule will be present at attoMolar concentration, different orientations of the molecule with respect to the hotspots give rise to peak shifts and selective enhancement in the SERS spectra. This also proves the ultrasensitive detection capabilities of Ni—Au/Pd superlattice sensor making it superior for detection of trace-level biomarkers.

The optimized sensor with maximum SERS efficiency was employed to study the ultrasensitive detection capabilities of superlattice in discovering EV signatures specific to glioblastoma. The limit of detection experiments was carried out using GBM EVs isolated from A172 cell line. The total EV concentration observed in the serum of glioblastoma patients is of the order of 106 particles/ml. However, the cancer-specific EVs and cancer stem cell-specific EVs will be present at very low concentrations in peripheral circulation. Besides, the concentration of tumor-derived EVs will be shallow at the early stages of glioblastoma, making them undetectable and necessitates ultrasensitive detection for a better prognosis.

The sensor was tested for its ultrasensitive detection capabilities by acquiring the SERS spectra at different EV concentrations. For this, the concentration of EVs isolated from A172 cell culture media was obtained from NTA analysis and was serially diluted in Milli-Q water to prepare a set of known concentrations ranging from 104 EVs/5 μL to 1 EV/5 μL. For analyzing the detection sensitivity, EVs were dropped on the sensor surface, and Raman spectral acquisition was carried out using a laser of 785 nm wavelength with 10 s exposure time.

FIG. 35B(iii) shows the SERS spectra of EVs at six known concentrations in a volume of 5 μL. The peaks at 928 cm-1, 1033 cm-1 and 1670 cm-1 show significant enhancement for all concentrations. These peaks correspond to the C—C stretching vibration of amino acids, phenylalanine, and Amide I proteins, respectively, indicating the binding of surface EV proteins to the sensor surface. The lowest limit of detection for EVs achieved so far is 100 EVs/10 μL as per the literature using 3D self-assembled nanopatterns. Ultrasensitive and direct detection of glioblastoma-derived cancer-specific EVs was achieved on the superlattice surface down to the lowest attainable limit of detection of 5 EVs/5 μL marking a 10-fold increase.

Interestingly, by virtue of 3D layered structure and its porous nature, the superlattice sensor can efficiently capture EVs on its surface. In addition, the quantum size of Ni cubes provides a high surface area to volume ratio for the interaction of EVs with superlattice surface, facilitating better adsorption and binding of EVs. Moreover, the nano ornamentation of 3D structure with Au/Pd boosts the enhancement efficiency by creating hotspots between the sharp edges of Ni quantum cubes and spherical Au/Pd nanoparticles. The enhanced electric fields generated due to localized surface plasmon resonance boost the EV signals, resulting in high detection sensitivity when the EVs are captured in these hotspots. This permits the quantification of EVs by the sensor at extremely low concentrations. The capturing of EVs by 3D layered Ni superlattice is also evidenced from HRSEM images (FIG. 35B(ii)).

The SERS-based label-free detection of EVs is an efficient approach since the technique allows single-molecule sensitivity with enhanced signals and a good signal-to-noise ratio without using specific antibodies. This proficient Ni-based superlattice sensor with high detection sensitivity for EVs, provides better prospects for clinical applications.

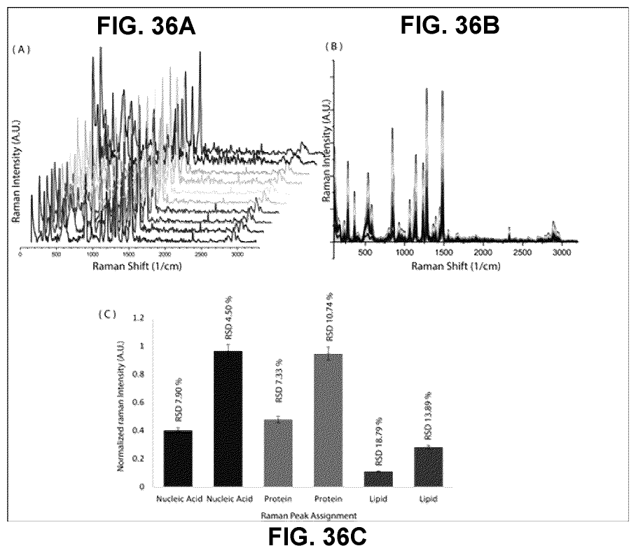

The SERS spectra of GBM EVs were also acquired from 10 randomly selected positions on the Ni—Au/Pd superlattice sensor to ensure the consistency and reproducibility of the EV biomarker spectral signatures FIGS. 36A and 36B. Most enhanced SERS peaks of EVs were identified. This selective enhancement depends on the surface bonding and interaction of EV particles with the superlattice sensor. Besides, the alignment of the molecules with respect to hotspots will also influence the peak intensities. Relative Standard Deviation (RSD) for the signature peaks were calculated to estimate the reproducibility. RSD was calculated for the peaks at 1235 cm-1 and 1281 cm-1 corresponding to proteins, 1063 cm-1 and 2888 cm-1 corresponding to lipids and 859 cm-1 and 1481 cm-1 corresponding to nucleic acids. The RSD values obtained were 7.3% and 10.1% for the protein peaks, 13.8% and 18.3% for lipid peaks and 19.8% and 4.5% for nucleic acid peaks respectively (FIG. 36C). All the RSD values measured were below 20% indicating distinctive SERS signal reproducibility.

Significant Variation in SERS Profiles of EVs Derived from GBM and GBM CSC Compared to EVs Derived from Non-Cancer Cells

The SERS spectra acquired on the Ni—Au/Pd superlattice sensor for all biological samples were baseline corrected, smoothened, and normalized using Spectragryph software. The as-obtained data was subjected to PCA analysis in Eigenvector software. In PCA, a total of 2367 (100 cm-1 to 1800 cm-1 and 2600 cm-1 to 3200 cm-1) variables were reduced to 10 principal components.

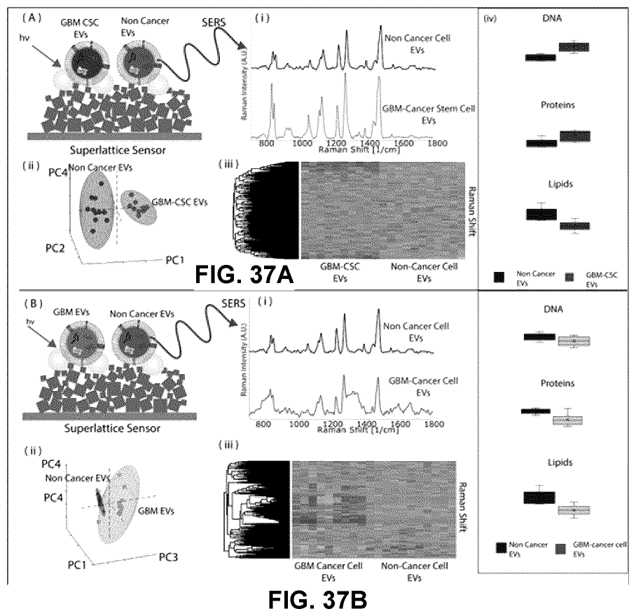

FIG. 37A shows the representative SERS spectra of normal cell EVs and GBM CSC EVs. The major peaks were assigned to lipids, proteins, and nucleic acids. It was observed that the relative intensities of all the dominant peaks were higher for GBM CSC EVd. Besides, to find the hidden molecular differences in the spectral signatures of EV biomarkers, the SERS spectra were thoroughly analyzed using principal component analysis (PCA). The EV signature spectra with a huge number of variables indicating a high dimensional data are reduced in dimensions by PCA to accentuate similarities or variances and thus to deduce patterns hidden in the heterogenous data. Hence, PCA analysis was employed as a tool to study how normal cell EVs would differ from GBM cancer cell and GBM cancer stem cell EVs at a molecular level.

A total of 10 spectra each from normal and GBM CSC EVs acquired from randomly selected points on the sensor was used for analysis. The graph plotted between the first three principal components is shown in FIG. 37A. Each point in the PCA plot corresponds to one spectrum. The red circles correspond to GBM CSC EVs, and the blue circles correspond to normal EVs. The points corresponding to spectra of normal EVs and GBM cancer EVs were clearly distinguished into two distinct groups in the reduced space. The enveloping ellipse is drawn for a confidence interval of 95%. The clustering indicates that some of the spectral features are unique for normal and GBM CSC EVs respectively either due to presence, or overexpression of certain proteins, lipids, and nucleic acids. The peaks that are responsible for the variance observed in PCA were validated by plotting a heatmap. FIG. 37B (iii) also shows the heatmap drawn for the spectra of normal EVs and GBM CSC EVs. The orange-red color shows a positive correlation while the green color depicts a negative correlation. The top band in the heatmap corresponding to the peaks 1490 cm-1, 1159 cm-1, 1073 cm-1 and 2887 cm-1 and the bottom band corresponding to peaks 1476 cm-1, 833 cm-1, 1288 cm-1 and 1506 cm-1 shows the maximum variance in the whole spectral data. The former four peaks were assigned to C—N stretching vibration coupled with in-plane C—H bending in amino radical cations, C—N stretching in proteins, Triglycerides/fatty acids, and CH2 asymmetric stretch of lipids and proteins respectively. The latter four peaks were assigned to Guanine and adenine of nucleic acids, asymmetric phosphate stretching in tyrosine, cytosine and phosphodiester groups in nucleic acids, and N═H bending of amino acids respectively. It is interesting to note that the EVs released by normal cells and GBM CSCs are of considerable difference based on its content. It can be deduced from these results that there are variations in the lipid, protein, and nucleic acid content of GBM CSC EVs when compared to normal cell EVs. For instance, it was reported that cancer cells express altered levels of lipids and cholesterol and are considered as a characteristic of aggressive cancers like GBM. This can be the reason for altered expression of lipid associated peaks in CSC-derived EVs. Of note, these results indicate that GBM CSC EVs can be a potential biomarker for diagnosis of Glioblastoma Multiforme due to the molecular level spectral differences it possesses as compared to EVs of non-cancer cells as shown in FIG. 37A(iv).

The PCA analysis between GBM cancer EVs and normal EVs also leads to a similar inference that spectral differences exist for these populations as well (FIG. 37B). The spectral features reduced to 3D space shows clustering for non-cancer and GBM EVs. However, the PC scores of the groups are not distant as seen for GBM CSC EVs. The PC plot shows that some spectral features can be common for non-cancer EVs and GBM EVs. However, differences exist for certain spectral bands as shown in the heatmap (FIG. 37B(iii)). The highest variance can be observed as a band in the middle formed by the peaks at 808 cm-1, 823 cm-1, 1358 cm-1 and 980 cm-1. These spectral bands showing distinct differences between non-cancer and GBM cancer were assigned to phosphodiester, out of plane ring breathing modes of tyrosine, Guanine, and C—C stretching of β-sheet in proteins. The expression of the listed molecules was lower in GBM cancer-derived EVs. Results revealed that the major differences in molecular content of non-cancer cell EVs and GBM cancer cell EVs were from the differential expression of proteins and nucleic acids it carried.

### Significant Variation in SERS Profiles of GBM Cancer Derived EVs and GBM CSC Derived EVs

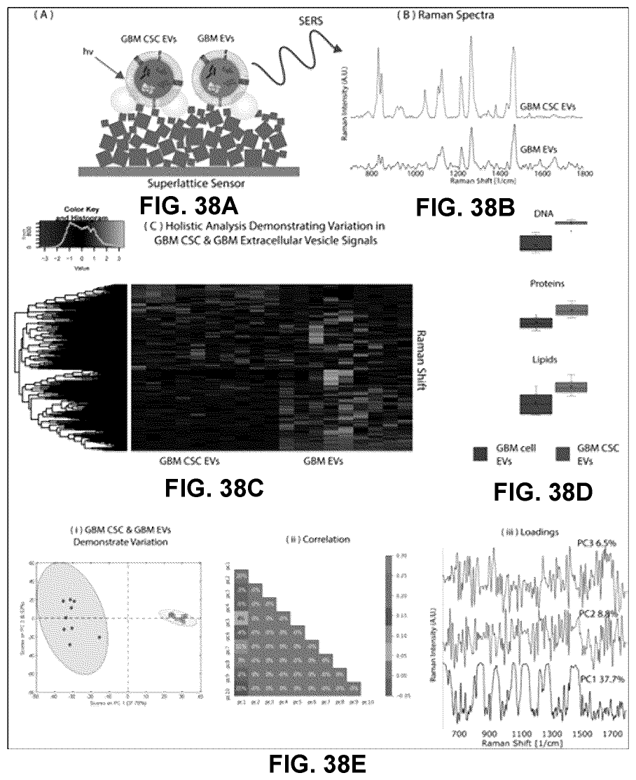

The EVs carry contents that are unique to their parent cells. FIG. 38A shows a schematic representation of EVs captured on superlattice surface. The heterogeneity in the Glioblastoma tumor cells can be studied by analyzing the EVs from cancer stem cell enriched population. The representative Raman spectra of GBM cancer and GBM CSC EVs are shown in FIG. 38B. Interestingly, there are visible spectral differences in the peak positions and intensities of these EV populations. The characteristic peaks correspond to vibrations of molecules of lipids, proteins, and nucleic acids. Although some peaks are common for both EV populations, there are certain peaks that are differently expressed in GBM CSC EVs. For instance, 802 cm-1 peak corresponding to uracil-based ring breathing mode is present only in the SERS signature of GBM CSC EV. Besides, intensity of all dominant peaks was higher in GBM CSC spectrum.

The SERS spectra of EVs derived from GBM cancer cells and GBM cancer stem-cell enriched population were characterized in detail by multivariate analysis to figure out peaks of maximum variance. Principal component analysis can find highly correlated variables and reduce the data into a set of eigenvectors/principal components that are linearly uncorrelated. These variables account for the highest variance in the SERS data of GBM cancer and CSC EVs. The PC plot for the first three principal components is shown in FIG. 38E(i). PC1 accounts for the maximum variance in the SERS data incorporating 37.7% of variance in the spectral data. Subsequently, PC2 and PC3 considers 8.8% and 6.5% respectively of the total variance. The positive values in the correlation plot indicate the principal components showing variances or the components that are linearly correlated. FIG. 38C shows a heatmap showing the spectral differences between GBM cancer and GBM CSC EVs. Highest variance was observed at 1497 cm-1, 862 cm-1, 1487 cm-1 and 1165 cm-1. These molecules are overexpressed in GBM CSC EVs indicated by the green bands associated with these peaks. Complementarily, they show a negative expression for GBM cancer EVs. These peaks were associated to C═C stretching in benzenoid ring of nucleic acids, phosphate group, Guanine, and tyrosine protein component. This implies that there are differences in the molecular components carried by cancer and CSC EVs, predominantly in DNA and RNA. The altered expressions in biomolecules are demonstrated in FIG. 38D after analyzing the normalized SERS intensities.

Biochemical Similarity of EVs Derived from GBM CSC with Parent GBM Tumor by SERS Profiling

The differences in SERS spectra observed for normal and cancer specific EVs proved that GBM cancer/CSC EVs can be employed as a biomarker to diagnose Glioblastoma Multiforme. However, our next challenge was to transform this potential biomarker to develop a rapid blood test. It is known that in case of Glioblastoma, tumor-derived EVs escape the blood-brain barrier and enter the peripheral circulation. The phospholipid bilayer of EVs protects the biomolecules it carries without degradation. The correlation of SERS spectra of GBM tumor tissue and EVs isolated from the serum sample of the same patient were analyzed to study the tumor specific characteristics expressed in EVs present in serum.

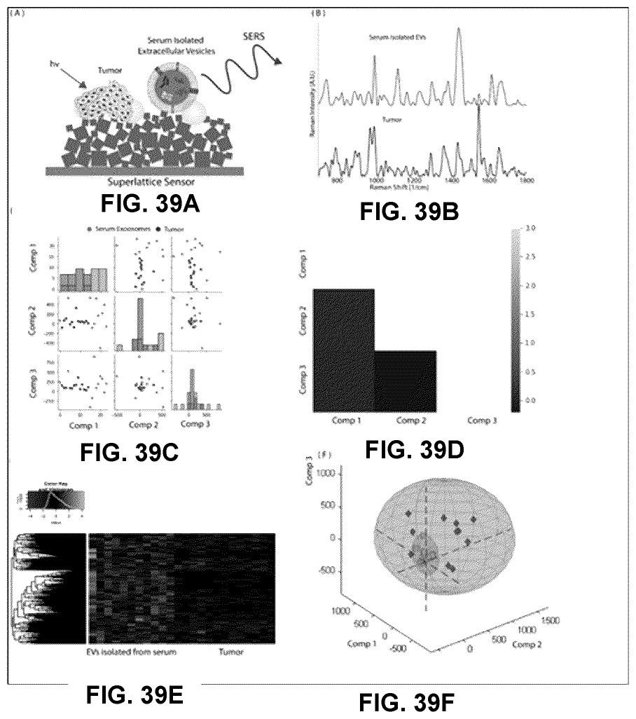

FIG. 39A shows a schematic showing clusters of tumor cells and EVs isolated from serum of corresponding patient adsorbed on superlattice surface.

FIG. 39B shows the representative SERS spectra of GBM tumor tissue sample and the serum derived EVs from the same patient. The sharp peaks originated from protein components like phenylalanine (1005 cm⁻¹), Amide 1 (1670 cm⁻¹), CH₂ bending of proteins and lipids (1446 cm⁻¹), nucleic acids (Guanine—1333 cm⁻¹), and from phospholipids (1745 cm⁻¹). FIG. 39C displays the pair plot obtained after PCA analysis for the first three principal components. The subplots in the diagonal show the distribution of principal components as a histogram. It can be observed that the points representing tumor and serum EVs are continuous rather than categorical, showing a high amount of similarity in their spectral signatures. These points therefore cannot be clustered into two distinct groups. The correlation plot in FIG. 39D shows that this similarity is highest between PC2 and PC3 indicating zero variance as evidenced from the pair plot. This result was also demonstrated in the PC plot. FIG. 39E shows a heatmap illustrating the spectral bands of high similarity. The PC plot shown in FIG. 39F displayed the PC scores of both serum EVs and tumor fall into the same ellipse in 95% confidence interval. Importantly, the result disclose that the serum EVs carry tumor characteristics.

To analyze the major peaks that contribute to the similarity between GBM serum EVs and GBM tumor tissue, heatmap was drawn for the SERS spectral data. The highest similarity observed were for the peaks at 1073 cm-1, 1255 cm-1, 1632 cm-1, 1513 cm-1, and 1376 cm-1. These peaks correspond to fatty acids, lipids, Amide I protein components, cytosine of nucleic acids, ring breathing mode of nucleic acids respectively. This proved that EVs carried characteristics of GBM tumor cells in the form of different biomolecules. In addition, results imply the necessity of considering the whole spectrum of biomolecules constituting EVs for a holistic analysis that can substantially improve diagnostic efficacy.

GBM Diagnosis with GBM CSC-EVs

The findings show that CSC derived EVs can be considered as a unique biomarker, and when combined with machine learning techniques can serve as a potential diagnostic platform for GBM diagnosis non-invasively. Only an efficient machine learning model can bring out the subtle differences in the SERS spectra facilitating prediction of GBM. To validate the usefulness of EVs as a biomarker reliable for clinical applications, the serum samples of 20 patients affected with Glioblastoma Multiforme for were subjected to PLSDA analysis. Unlike PCA which is unsupervised, PLSDA is an effective supervised machine learning model which can predict and classify the data into distinct groups based on the similarities and covariances in the data.

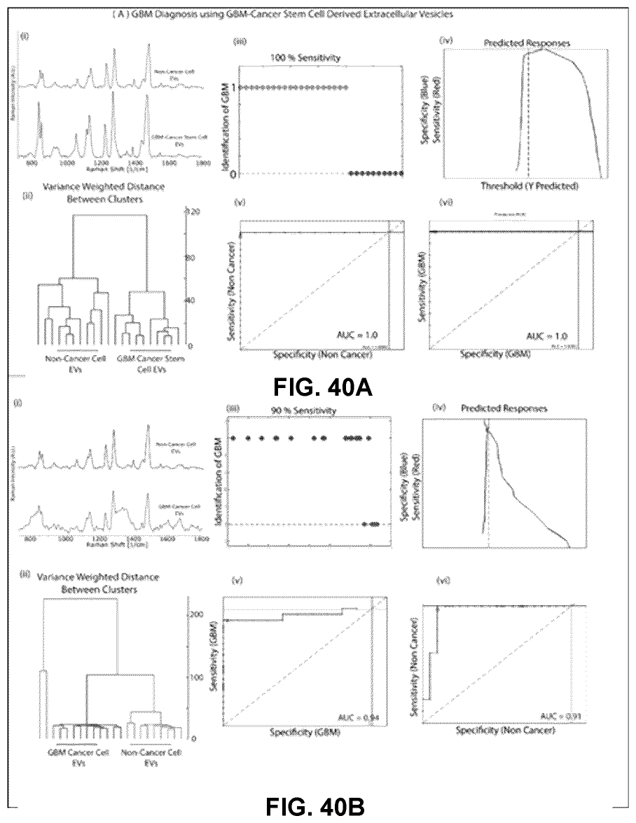

The SERS spectra of serum samples were obtained and were preprocessed before loading to the PLSDA model. The PLSDA model was executed in three steps; i) training using normal cell-derived EVs, GBM cancer cell derived and GBM CSC EVs, ii) validation of model using 20% of training data, and iii) testing using the patient serum samples. In the training phase, the machine learning model was first trained using normal EVs and GBM cancer derived EVs. The cross-validation of the model confirmed an accuracy of 100%. FIG. 40B ii shows the dendrogram obtained after hierarchical clustering of training data. Although all cancer cell EVs fall into a single cluster, two of the dataset of non-cancer EVs were considered as outliers. The validated model was then used for testing the data obtained from patient serum samples. Plasma spiked with non-cancer cell EVs was used to simulate serum composition from healthy individuals. FIG. 40Biii corresponds to the prediction probabilities with which each set of samples were categorized. The purple circles indicate GBM patients, and the green circles indicate healthy individuals. A probability of 0 indicates a healthy individual and a probability of 1 indicates GBM patient. Detection threshold was set to 0.5 and the prediction test displayed a sensitivity of 90% and a specificity of 100%. The Receiver Operating Characteristic (ROC) curve is shown in FIG. 40B v and vi for GBM and healthy populations respectively. Area under the curve (AUC) calculated was >0.9 implied that it is an efficient diagnostic tool.

However, to further improve the prediction accuracy, GBM CSC derived EVs were employed in the training phase to incorporate the intratumoral heterogeneity of GBM. The hierarchical clustering of the training data is shown as a dendrogram in FIG. 40. The training data presented a proper clustering of normal and GBM CSC EVs. Besides, the Euclidean distance between the datasets within a cluster is shorter than the dendrogram in FIG. 40B ii. This indicated less variance within the spectral data of EV subpopulations which subsequently was employed to construct a better model for diagnosis.

Interestingly, the model trained with GBM CSC derived EVs resulted in 100% sensitivity and 100% specificity for Glioblastoma Multiforme prediction (FIG. 40 A iv). The cut off was set to a probability of 0.5. The predicted probabilities are demonstrated FIG. 40 A iii. The green circles denote healthy individuals, and the red circles denote GBM patients. The ROC curves are shown in FIG. 40 A v and vi. The AUC was 1 for prediction of healthy as well as GBM cancer population. Results manifested 100% accuracy in the binary classification presenting a potential EV based diagnostic platform for prediction of Glioblastoma Multiforme.

### Diagnosis of Glioblastoma Using Cell Free DNA

This study introduces an ultra-sensitive and non-invasive methodology of GBM detection directly from patient blood samples using GSC-DNA as a reliable biomarker and 3D plasmonic meta sensors with a sub-single molecule sensitivity. The GSCs account for the heterogeneity, tumour progression and the aggressiveness of the cancer and hence, plays a vital role in the detection mechanism. The trace levels of the GSC associated ctDNA were detected by SERS of the samples on the 3D plasmonic sensors. The high level of sensitivity of the sensors were achieved by adapting the following: Size reduction of the carbon nanoparticles that activates the organic plasmons, arrangement of metasensor in a 3D cluster that entraps the analyte and increases the surface area of adsorption and incorporation of functional groups on the meta sensors that enhances the binding of the analytes to the sensors. The detection was achieved by using machine learning algorithms for data analysis. SERS profile of the in-vitro GBM DNA and GSC DNA are the data collected and used for training the machine learning algorithm to detect the differences between the GBM and GSC DNA. These differences were utilized to detect the cancer. The tumor derived DNA from GBM patient samples were used as the testing data to accurately pinpoint the differences and group them as healthy and cancerous samples. SERS data obtained form 5 μL of GBM patient's peripheral blood sample on the meta sensor platform was used as the validation data for the test. The classification accuracy and specificity were increased up to 96.9% and 94.4% respectively by incorporating the GSC-DNA along with the GBM DNA.

### Detection of the Differences Between the In-Vitro Healthy Fibroblast and GBM Cancer Cell Derived DNA

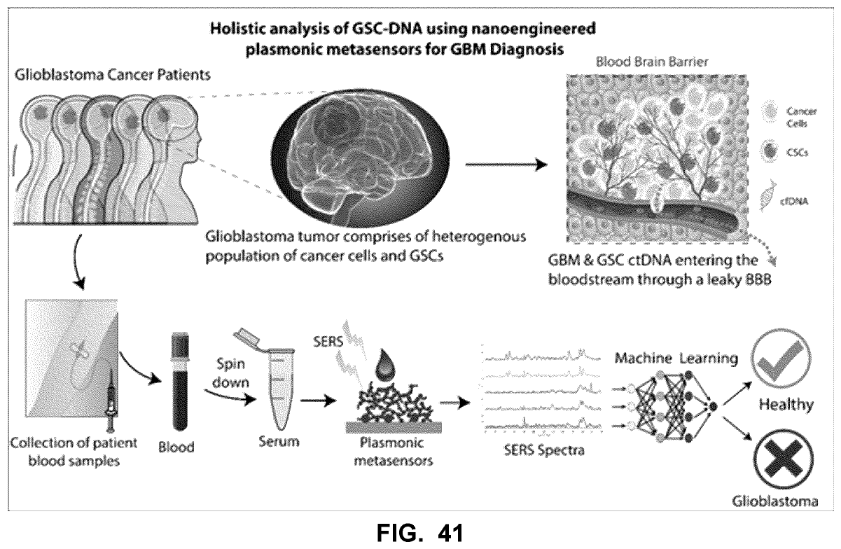

FIG. 41 shows a schematic representation of holistic analysis of GSC-DNA for GBM diagnosis.

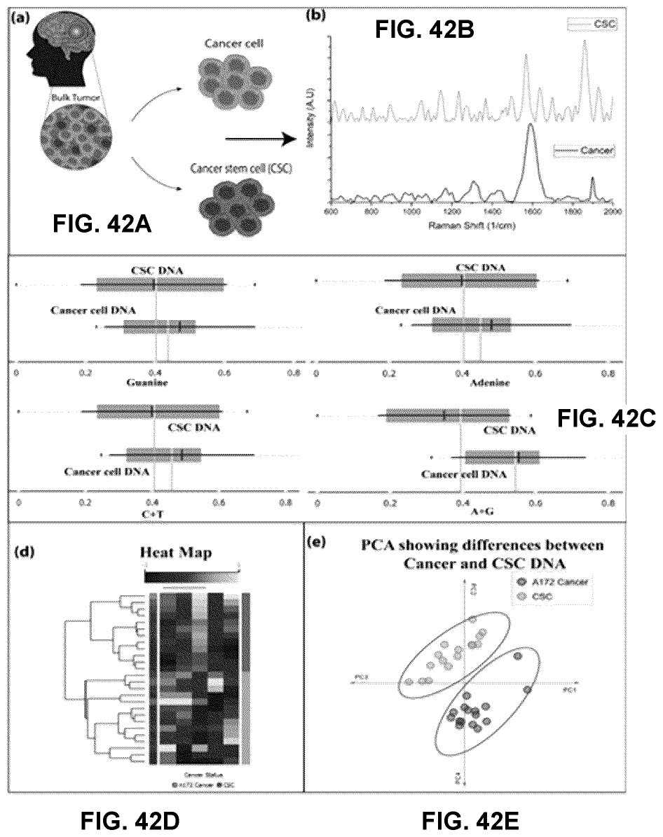

The SERS spectra of the DNA isolated from in-vitro glioblastoma cancer cells (A-172) and healthy fibroblast cells (WI-38) were recorded to study the differences exhibited by them. The PCA of the SERS spectra of the cell derived DNA showed a clear distinction between the glioblastoma and healthy cells. The predominant peak assignments that are responsible for the variance causing the distinct classification between the healthy and cancer cells were obtained from the PC loading curves of all the principal components considered for the analysis. Here in this case, four principal components have been identified and used for the PCA. FIG. 42 shows the classification of the obtained data into healthy and cancerous cells. FIG. 42A shows a bulk tumour consisting of heterogenous populations from which DNA is isolated and data is gathered. The SERS spectra of the healthy cell DNA and glioblastoma DNA is shown in FIG. 42B. Heatmap of the data shows the correlation of all the similar spectra and clear distinction between the healthy cell DNA and the glioblastoma cell DNA in FIG. 42D.

FIG. 42D shows the principal component analysis of the data showing the classification of the spectral data. FIG. 42F shows the PC loading plots of the four principal components used for the PCA. The prominent peak variances are observed at 767 cm⁻¹, 971 cm⁻¹, 1048 cm⁻¹, 1049 cm⁻¹, 1066 cm⁻¹ and 1076 cm⁻¹ which corresponds to the characteristic peak values of the nucleic acid bases and amino acids namely Thymine (T), Cytosine (C), Guanine (G), Glycine, L-Valine and L-Tryptophan ²⁸ respectively. This PC loading peak variance at the nucleic acid catabolites such as the nitrogenous bases and the amino acids reveals the cancerous nature of the cell from which the DNA is isolated.

Dysregulation in the expression of proteins or the expression of defective proteins is a significant feature of cancer cells. The variance at the peak positions of the PC loading plots at the amino acid peaks shows that the SERS spectra of the DNA compared, shows significant differences in their gene expression patterns^{29,30}. The variance observed at the 756 cm-1 in the PC4 and the 1075 cm-1 peak in the PC2 and PC3 corresponds to the out of plane ring mode and the in-plane ring breathing of the suggestive oncoprotein c-MYC^{29,30} which again is a contributing factor for classifying the SERS spectra of the DNA isolated from healthy and cancer cells.

### Differences Exhibited by the Glioblastoma Cancer DNA and the Glioblastoma Cancer Stem Cell DNA

Glioblastoma is an aggressive form of cancer, and at higher stages, it relapses and metastasizes to other locations. The presence of the rare population of cancer stem cells (CSC) is the reason for metastasis and the aggressiveness of the cancer. So, it is highly imperative to take into account the scarce CSC population for the diagnosis of glioblastoma. But the problem associated with using CSC is that it is significantly less in number and the CSC-associated cell-free DNA in the patient blood/serum is at a deficient concentration making it hard to detect. To account for tumour heterogeneity, the SERS spectra of the DNA isolated from in-vitro glioblastoma cells (A-172) and the cancer stem cells derived from A-172 were used to unique spectral signatures that differentiate them into distinct populations based on the differences in their DNA and proteins.

The DNA of the glioblastoma cancer cells and CSCs were dropped on the nanodiamond sensors and their SERS spectra were recorded and analyzed to understand the differences exhibited by the DNA signatures. FIGS. 42A and 42B show the peak assignment differences between the spectra. The DNA spectra of both GBM derived DNA and GSC derived DNA comprised of abundant contributions from the nucleobases of the nucleic acids. The prominent peaks observed were at 725 cm⁻¹ and 1304 cm⁻¹ corresponding to the characteristic peaks of Adenine^{28,31}, 782 cm-1 corresponding to the PO₂ from the phosphate backbone of the DNA ^{28,31}, 1059 cm⁻¹ that is a characteristic peak of the C—O stretching of the acidic group from the DNA³², 1448 cm⁻¹ corresponding to the CH₂ deformation^{33,34}, 1319 cm⁻¹ corresponding to Guanine²⁸, and the three peaks at 1339 cm⁻¹, 1479 cm⁻¹ and 1581 cm⁻¹ are attributed to the combination of adenine and guanine (A+G)^{28,31,35,36}.

FIG. 42C shows the box plots of the Raman intensities at the A, G and C+T peak positions of both GBM DNA and GSC DNA. The Raman intensities of the GSC DNA appears to be more stable. The interquartile range within which 50% of the middle scores of each group falls, for GSC DNA is greater than that of the GBM DNA. This shows that the GSC DNA data is more stable and reliable with a higher interquartile range spanning a wide limit of variance.

FIG. 42D shows the heat map of the negative correlation between the GBM DNA and GSC DNA spectral signatures. The yellow and blue bands are the extreme values and represent the two groups of DNA considered here. The separate yellow and blue colored bands at the ends of the heatmap is suggestive that the two groups of DNA exhibit significant and distinct differences, despite the similar DNA features being expressed as mixed colour bands in the middle. FIG. 42D shows the PCA plot of the DNAs from GBM and GSCs. They are clustered into 2 individual groups without overlapping. Though both the data contains DNA features from the cells, the principal components identifies the peak positions that contribute to the rich differences exhibited by the two groups of DNA. FIG. 42E shows PCA scatter plot showing the differences and their clustering.

### Quantification of the GSC DNA Concentration in the Serum by Prediction

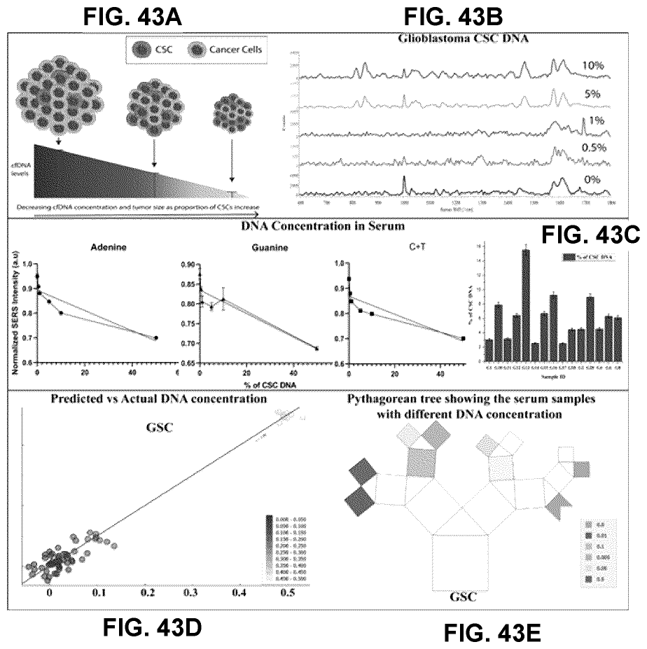

The concentration of CSC DNA in blood is directly related to the size and or stage of the tumor. The GSC DNA was serially diluted into many concentrations along with an optimal concentration of healthy fibroblast cell free DNA. This is to mimic the plasma DNA levels which contains both healthy and cancer DNA. FIG. 43A shows the SERS Spectra of the GSC DNA diluted at various concentrations from 10 to 0% and the metasensors were able successfully able to record signals even at the lowest concentrations. FIG. 43B shows the regression analysis of the SERS intensities of the GSC DNA at the C+T, A and G characteristic peak positions.

They all follow a linear regression pattern suggesting that they are detectable up to the lowest concentrations. FIG. 43C shows the amount of CSC DNA predicted in the given patient samples according to the stage of cancer.

Using the line of regression equation, the actual concentration of the CSC DNA present in the given samples were calculated and is shown in the scatter plot in FIG. 43D. They clearly form groups between the different levels of DNA concentration in the blood plasma. Also, FIG. 43E shows the Pythagorean tree, showing the relationship between the GSC DNA concentration and the serum samples and their grouping together based on the DNA concentration. Even though the spectral features are similar to each other in different concentration, they still form individual clusters which gives us the ability to quantify the DNA levels and the contributing factors to the disease in the blood.

Correlation Between GBM DNA, GSC DNA, Patient Tumor Derived DNA and the Serum Samples Enabling the Direct Detection of GBM from Patient Serum Samples

SERS spectral data of the DNA isolated from the tumour biopsy of the glioblastoma patients was used to establish its resemblance to the cell-free DNA present in the patient serum samples. The similarity between the glioblastoma cell-derived DNA and patient tumour-derived DNA was also demonstrated by comparing their SERS spectra. The SERS spectral data of the DNA isolated from glioblastoma cell-derived and tumour-derived DNA are compared to study the similarity patterns. But the glioblastoma cell DNA showed similarity only to a few data points. Glioblastoma is an aggressive type of cancer, and the tumors consist of a heterogeneous population of cancerous cells, healthy cells and a rare population of cancer stem cells (CSC). The aggressiveness of the cancer is attributed to the presence of CSCs in the tumour. Therefore, the presence of CSC DNA must be accounted for in the DNA isolated from the tumour^{37,38}. The lower similarity index of the cancer cell DNA and tumour DNA could be because of missing out on the other populations of the cells. Therefore, DNA was isolated from the in-vitro glioblastoma CSCs, combined it with the cancer cell DNA, and compared it with the tumour DNA.

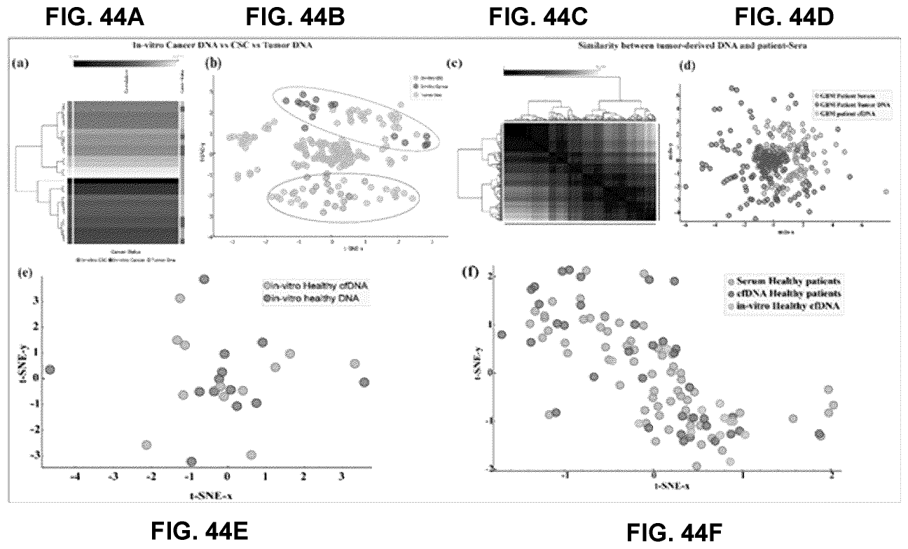

FIG. 44A shows the heatmap of the data similarity between the cancer cell-derived DNA, CSC-derived DNA and tumour-derived DNA. All the three are correlated together, and the correlation between them ranges from negative 0.406 to 0.2775. FIG. 44B shows the t-distributed stochastic neighbor embedding (tSNE) clustering plot of the three data being clustered into three overlapping clusters. The tumour DNA (green dots) is found in more similarity with the CSC-derived DNA (blue dots) and cancer cell-derived DNA (red dots). An intermediate cluster also hasn't shown any apparent similarity with either cancer or CSC cell-derived DNA, which could be attributed to the other components of serum such as the serum proteins, cholesterol and pigments.

The established similarity of tumour-derived DNA to the CSC and cancer cell-derived DNA is extended to show their holistic similarity to the cell-free DNA in patient sera. FIG. 44D shows the multi-dimensional scattering plot of the similarity analysis between the tumor-derived DNA GBM patient serum samples and patient derived cfDNA isolated from the serum. They are clustered at significant number of points demonstrating the existent similarity between the tumor DNA and the cell-free DNA in the serum samples of the respective patients. The cell-free DNA that escapes the blood-brain barrier in the case of a glioblastoma patient is a highly scarce and tracing them in the patient serum at such low concentrations is the issue faced in using the liquid biopsy for the detection of cancer. The cfDNA has to be isolated and then amplified by using PCR and then used for studies. But here, the cfDNA in the serum samples was detected from the patients at a very low concentration and correlated it with the respective tumor DNA samples, thereby achieving an ultrasensitive detection of the cfDNA from the patient sera. FIG. 44C shows the heatmap that supports the similarity between the tumor-derived DNA and the patient serum.

In order to establish the distinction between the serum of a healthy individual and a GBM patient, the spectra of serum samples was obtained from 10 healthy individuals and respective cfDNA were isolated and SERS spectra was obtained. The expression of serum properties in cfDNA was stated by the similarity analysis. FIG. 44E shows the tSNE analysis between the serum and corresponding cfDNA isolated from the serum samples and they display a high similarity between them which states that the cfDNA exhibits the serum properties. In addition to this, the correspondence of the healthy serum and cfDNA samples with the cfDNA isolated from in-vitro healthy fibroblast cells was checked. FIG. 44E clearly shows the properties of cfDNA and serum from healthy individuals closely reflecting the spectral characteristics of the cfDNA from in-vitro healthy cells. This makes it evident that the healthy serum samples doesn't contain any information related to GBM and has unique signatures.

### Holistic Analysis of GSC-DNA for Diagnosis of Glioblastoma Using the Nanoengineered Plasmonic Metasensors

Glioblastoma is hard to detect in its early stages because the characteristic biomarkers of this aggressive form of brain cancer cannot extravasate the blood-brain-barrier³⁹. At the later or complex stages, there develops a leaky BBB that allows the biomarkers like the cell free DNA and apoptotic cells to cross the BBB and enter the blood stream. The concentration of this biomarkers in the blood is insufficient for the direct detection of disease by any methods available. The plasmonic metasensors used in this study shows enhanced Raman efficiency that makes it a suitable candidate to detect the glioblastoma from patient blood serum^{40,41}.

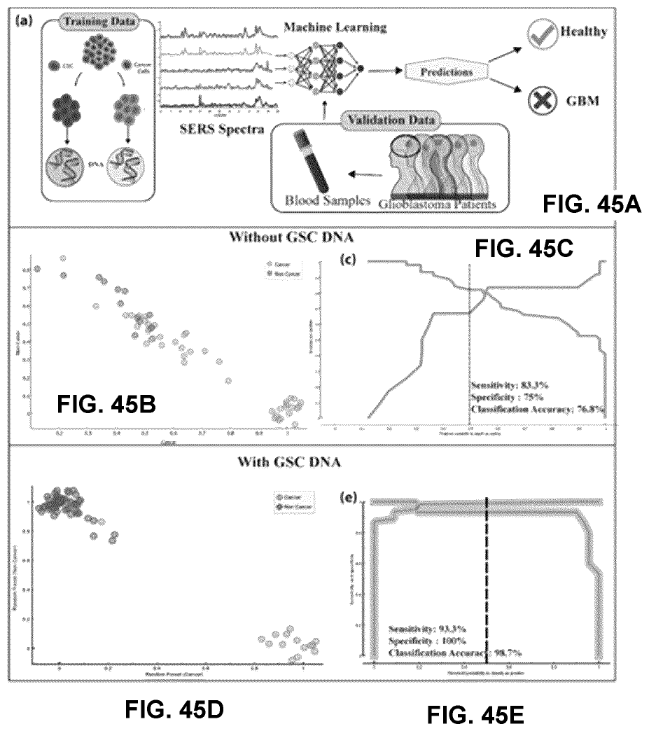

The DNA derived form the GBM and the GSC are used as the training data for the machine learning to train the algorithm for GBM diagnosis. The Raman spectra of patient serum is the validation/test data which is diagnosed. FIG. 45A shows the illustration of the GBM diagnosis from patient blood sample using the plasmonic metasensors and machine learning algorithm. FIG. 45B shows the scatter plot depicting the classification of the patient serum samples into healthy and glioblastoma. It is obvious from the scatter plot that the classification is not accurate enough and there is a high degree of overlap between the two groups of samples. FIG. 45C shows the specificity/sensitivity curve. When the algorithm was trained only with the GBM derived DNA, the sensitivity and specificity for the GBM positive samples were 83.3% and 75% respectively and the classification accuracy was only 76.8%. This clearly suggests that GBM DNA is alone not an ideal training dataset required to diagnose a hard to detect cancer like glioblastoma.

Glioblastoma tumour is a highly heterogenetic population of cells and therefore to account for the heterogeneity, the GSC DNA data was also combined with the GBM data as a training dataset to train the algorithm. FIG. 45D is the scatter plot showing the classification of the two groups of samples into heathy and cancer. The classification of the data is much better and distinct with a very less overlap of the data points. FIG. 45E shows the sensitivity/specificity curve. When the GSC DNA data was combined along with the GBM DNA data as the training set, the classification accuracy greatly improved to 98.7% and the sensitivity and specificity were 93.3% and 100% respectively.

### Prediction of Tumor Location in Brain Cancer Using Raman Molecular Profiles of Patient Serum

### MLP-LIBT for Prediction of Tumor Location

This study designed a fully connected Artificial neural network that can act as a location identifier for brain tumor. The Multilayer perceptron used here is a fully connected artificial neural network that works on feed-forward mechanism. The model is composed of a three layer dense network with nodes or neurons that act as processing elements that use linear activation and backpropagation for the training of the model. The input data was the whole SERS spectrum of serum samples that had 2573 variables or Raman values along with their brain tumor locations. The output layer generates the classification based on the input data and the computations from the hidden layer. At each layer, the predicted output is compared with the actual output. When there is a high error value, the weight corresponding to the variable is recalculated to improve network performance. The training process continues until a good validation accuracy is obtained. Testing employs a new dataset for which the network is unaware of actual values. Here, in this study the output was brain tumor locations that are classified to nine namely, cerebellar, left frontal, left temporal, left occipital, left parietal, right frontal, right temporal, right occipital, and right parietal.

The pre-processed data after normalization was augmented to generate a dataset of 3000 SERS spectra. The augmentation of data helps in eliminating the probabilities of overfitting. When there is a small amount of data the pattern recognized by the model is often not capable of recognizing or classifying a new data. This makes the model use irrelevant data for classification. This is eliminated by constructing an augmented database by a number of random transformations. This data was given to the input layer as input and used to train the MLP ANN model. The learning rate was set to 0.01 to ensure the model captures details on every single peak of Raman spectra. In the MLP algorithm, the output of the first layer acts as the input of the next layer and all layers in the network were fully connected. However the functions or parameters such as weights are independent of the layers. Essentially the weights assigned to variables in each layers are unique. These weights are assigned randomly and then adjusted by the backward propagation by estimating the error. The summation of weighted outputs in one layer is fed to the next layer as input through an activation function. The activation function employed here is Rectified Linear Activation (ReLu) which maps the input to the output by removing the negative values at each step.

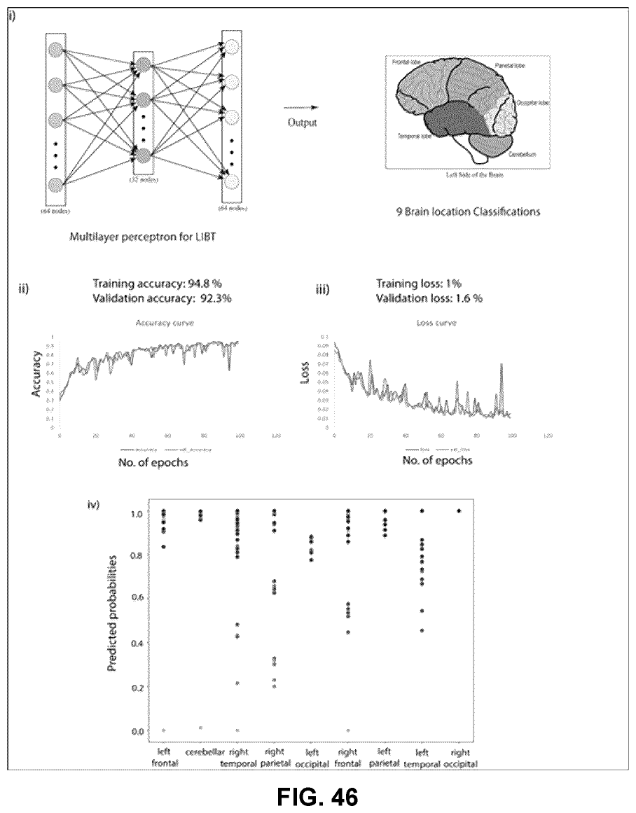

After training, the validation was done using 10% of the data and loss and accuracy curves were monitored for increasing epochs. The total number of epochs was set to 100 were a maximum validation accuracy of 94% was obtained. The number of epochs were capped to ensure there is no overfitting of the model. The prediction results are given by the output layer. To observe the loss Mean Squared Error (MSE) function was used. The MSE takes the difference between the actual and predicted value, square it and takes average for the whole dataset. The loss function continuously monitor the performance of the algorithm over increasing epochs and optimize the fitting process. A low loss value obtained as shown in FIG. 46 indicates accurate classification or prediction of tumor locations thus generating realistic solutions. Network training stops when the error is increasing for the validation dataset. At the end, another dense layer was used with a softmax activation function in order to decide in which class the data belongs to. The softmax output layer contains nice output target for each of the locations where the data is trained. The model performance was evaluated by feeding in the testing data. For testing the MLP algorithm, 10% of the augmented data was utilized. The values used in a testing set have no effect on training and so provide an independent measure of the network performance after training. Testing set of data generates independent accuracy and precision values which demonstrates the efficiency of the network. The Location identifier for brain tumor (LIBT) predicts the output as a classification system of 9 outputs. The 9 output locations were set as cerebellar, left frontal, left temporal, left parietal, left occipital and right frontal, right temporal, right parietal and right occipital. The output was taken as number of actual brain tumor locations and number of predicted brain tumor location, along with accuracy, precision, recall and F1 score to measure the performance of the model. The accuracy values with which each brain tumor locations are predicted from the serum SERS spectra is presented in Table 7. The accuracy ranges from 87% to 100% for different tumor locations. Other parameters obtained such as precision, recall and f1 score is listed in Table 8, below.

TABLE 6
Model Details

| Hyperparameter | Configuration |
|---|---|
| Number of hidden layers | 3 |
| Number of nodes per layer | (64, 32, 64) |
| Activation function | Rectified Linear Unit (ReLu), SoftMax |
| Learning rate | 0.01 |
| Optimizer | Adam |
| Loss function | Mean Squared error (MSE) |
| Number of training epochs | 100 |

TABLE 7
Brain Tumor Location Prediction Results

| Sample | No. of actual | No. of correctly predicted | |
|---|---|---|---|
| location | locations | location | Accuracy |
| Cerebellar | 73 | 70 | 95.89% |
| Left frontal | 98 | 85 | 86.73% |
| Left occipital | 10 | 10 | 100% |
| Left parietal | 19 | 19 | 100% |
| Left temporal | 20 | 19 | 95% |
| Right frontal | 144 | 142 | 98.61% |
| Right Occipital | 13 | 13 | 100% |
| Right parietal | 42 | 37 | 88.1% |
| Right temporal | 71 | 69 | 97.18% |

TABLE 8
Accuracy and Precision of the Prediction Model

| Sample location | Precision | Recall | F1 score |
|---|---|---|---|
| Cerebellar | 0.99 | 0.96 | 0.97 |
| Left frontal | 1.00 | 0.87 | 0.93 |
| Left occipital | 0.91 | 1.00 | 0.95 |
| Left parietal | 1.00 | 1.00 | 1.00 |
| Left temporal | 0.95 | 0.95 | 0.95 |
| Right frontal | 0.90 | 0.99 | 0.94 |
| Right Occipital | 1.00 | 1.00 | 1.00 |
| Right parietal | 0.88 | 0.88 | 0.88 |
| Right temporal | 0.96 | 0.97 | 0.97 |

### Construction of Weighted Raman Co-Expression Network

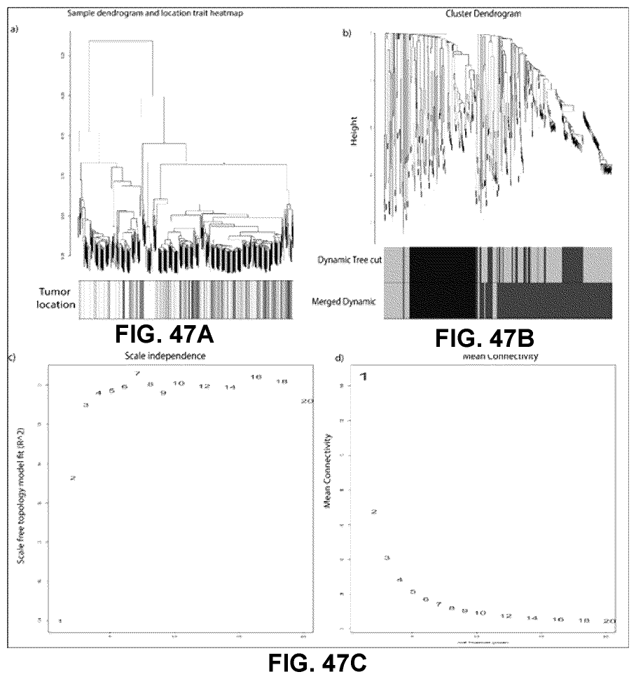

FIG. 47A shows a dendrogram clustering Raman data based on the tumor location trait to detect the outliers.

FIG. 47B shows a cluster dendrogram generated to identify modules of highly correlated Raman peaks.

FIG. 47C shows a scale free topology model—determination of soft-threshold power based on the fit.

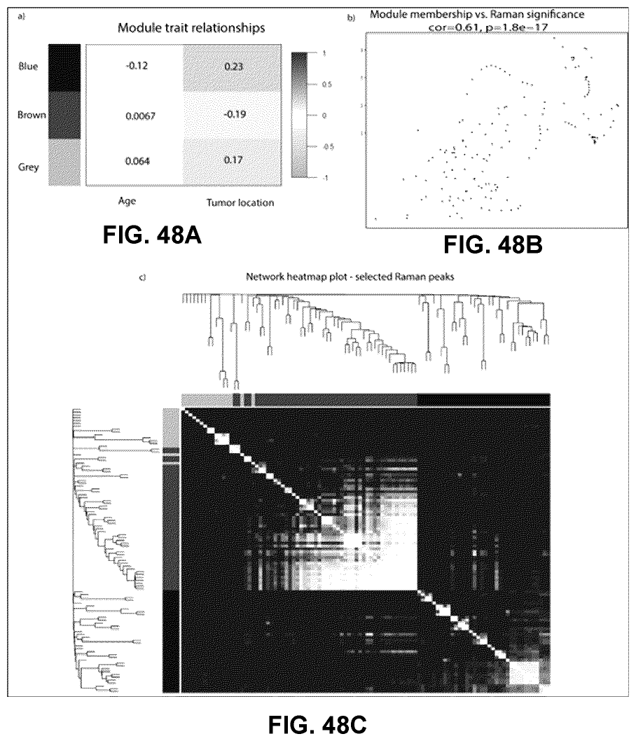

FIG. 48A shows a correlation plot showing the module-trait relationship—Blue module shows highest positive correlation for location trait.

FIG. 48B shows a scatter plot showing Raman significance of blue module (p=1.8×10⁻¹⁷) for location trait.

FIG. 48C shows a network TOM heatmap plot for selected Raman peaks. The plot shows the degree of correlation within each module.

The SERS spectra of serum samples of brain tumor patients were acquired using the nanosensor and the data was augmented to overcome the limitations posed by a small dataset. The augmented data having whole SERS spectrum accounting for 2573 variables/Raman peaks were given as input for the Weighted co-expression network analysis. A sample dendrogram was constructed to detect the outliers and find similarity in dataset. The heatmap in the FIG. 48 shows how the dataset is corelated with the clinical trait-location. The as-generated similarity matrix was converted to an adjacency matrix. To ensure scale-free topology, the soft threshold power was set to β=6. The 1-TOM value was considered as a factor to account for the similarity matrix and to construct a hierarchical clustering. Co-expressed Raman peaks were merged into modules using the dynamic tree-cut procedure (FIG. 48). A total of 3 significant modules were identified after merging similar modules (Blue, Turquoise and Cyan). Raman peaks that are not classified into any modules were merged as the Grey module.

Identifying Significant Raman Signatures Associated with Brain Tumor Location

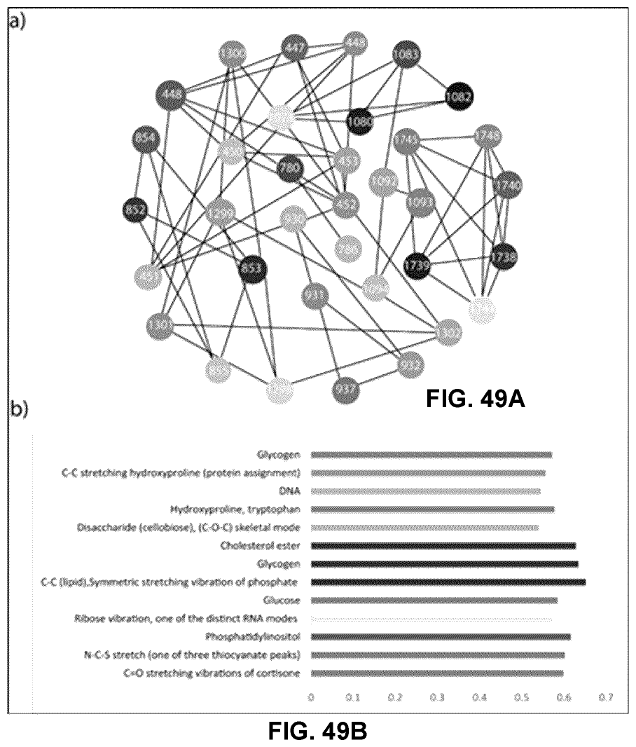

FIG. 49A shows a network of Raman peaks showing highest correlation with location trait. All peaks in the network expressed a weight above 0.6.

FIG. 49B shows as bar chart depicting Raman peak assignment corresponding to most significant peaks based on their weights.

The modules were then correlated with the tumor location trait. The correlation cut-off value was set to p=0.05 and major three modules were considered. Among them, the blue module was identified to be most positively correlated with the location trait (FIG. 49). This module was used for all further analysis and to identify Raman peaks that are significantly correlated to the location trait. The Raman significance versus Module membership was plotted for the blue module shown in FIG. 49. A statistically significant correlation for blue module with the Raman peaks associated with tumor location (p value=5×10 (−7)) was obtained. These Raman peaks were ranked according to their Raman significance values. The highly correlated Raman peaks are listed in Table. The most significant peaks with an associated weight value higher than 0.6 was visualized using the Cytoscape software. The nodes represented in FIG. 49 and their corresponding Raman assignments are listed in Table.

TABLE 9
The nodes represented in FIG. 49 and their
corresponding Raman assignments.

| Peak | Raman peak assignment | Weight |
|---|---|---|
| 1740 | C═O stretching vibrations of cortisone | 0.598998 |
| 448 | N—C—S stretch (one of three thiocyanate peaks) | 0.602049 |
| 780 | Phosphatidylinositol | 0.61609 |
| 1746 | Ribose vibration, one of the distinct RNA modes | 0.574232 |
| 937 | Glucose | 0.584597 |
| 1082 | C—C (lipid), Symmetric stretching vibration of | 0.651734 |
| | phosphate | |
| 853 | Glycogen | 0.634265 |
| 852 | Cholesterol ester | 0.62792 |
| 1094 | Disaccharide (cellobiose), (C—O—C) skeletal mode | 0.539802 |
| 1093 | Hydroxyproline, tryptophan | 0.577505 |
| 930 | DNA | 0.545191 |
| 1092 | C—C stretching hydroxyproline (protein assignment) | 0.556187 |
| 1300 | Glycogen | 0.571532 |

### Significance of Using SERS Whole Spectrum for Tumor Location Prediction

The Weighted Raman co-expression analysis revealed that proteins (glycogen, tryptophan), lipids (cholesterol ester) and nucleic acid peaks significantly correlate with the brain tumor location trait. Among these the major biomolecules identified were glycogen, cortisone, phosphatidylinositol, cholesterol ester, DNA and several RNA modes. A summary for each biomarker is given below and would contribute to further research and experiments that can validate our studies and lead to novel ways of diagnosis, prognosis and treatment of brain tumors.

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

What is claimed is:

1. A method of providing a cancer assessment for a patient, the method comprising:
isolating a volume of a fluid from a fluid sample of the patient, the volume of fluid including at least one biomarker;
adding at least a portion of the volume of fluid to a nanosensor, the nanosensor comprising nanoparticles configured to capture the at least one biomarker and amplify signals emitted by the at least one biomarker during Raman spectroscopy;
performing Raman spectroscopy on the volume of fluid on the nanosensor to produce a sample Raman spectrum, the sample Raman spectrum having amplified signals indicating the presence of the at least one biomarker on the nanosensor;
processing the sample Raman spectrum using data from template Raman spectra from known cancer samples having cancer characteristics to detect whether the sample comprises one or more of the cancer characteristics; and
based on the detected one or more cancer characteristics, providing the cancer assessment of the patient.

2. The method of claim 1, wherein the one or more cancer characteristics of the sample are detected based on determining which correlation values obtained by correlating the amplified signals of the sample Raman spectrum to template Raman spectra from the known cancer samples having the cancer characteristics are larger than a correlation threshold.

3. The method of claim 1, wherein the one or more cancer characteristics of the sample are detected by:
performing feature extraction on the Raman sample spectral data to extract feature values;
performing classification by applying the feature values to at least one set of classification models determined for the at least one biomarker to detect the one or more cancer characteristics; and
providing the cancer assessment by incorporating each of the detected cancer characteristics,
wherein the classification models are determined using the template Raman spectra from the known cancer samples.

4. The method of claim 3, wherein the feature extraction is performed using Principal Component Analysis, Multivariate Curve Resolution Analysis or a combination thereof.

5. The method of claim 3 or claim 4, wherein the classification model is one of Partial Least Squares Discriminant Analysis (PLSDA), Support Vector Machine Discriminant Analysis (SVMDA) and Artificial Neural Network analysis (ANN) TSNE and Random Forest classification.

6. The method of any one of claims 1 to 5, wherein the cancer characteristic is a cancer type, a cancer stage, cancer metastasis, cancer potential for metastasis or a combination thereof.

7. The method of any one of claims 1 to 6, wherein the biomarker is a cancer cell, a cancer stem cell or a cancer initiating cell.

8. The method of any one of claims 1 to 6, wherein the biomarker is one or more extracellular vesicles.

9. The method of claim 8, wherein the biomarker is one or more extracellular vesicles of circulating cancer initiating cells (CICs) or cancer stem cells.

10. The method of any one of claims 1 to 6, wherein the biomarker is a cell-free nucleic acid of cancer, cancer initiating cells (CICs) or cancer stem cells.

11. The method of claim 10, wherein the cell-free nucleic acid is as cell-free DNA.

12. The method of claim 11, wherein the cell-free DNA is molecularly modified by one of methylation, oxidation and acetylation.

13. The method of claim 10, wherein the biomarker is structure and molecular composition of cell-free DNA.

14. The method of any one of claims 1 to 6, wherein the biomarker is serum.

15. The method of any one of claims 1 to 6, wherein the biomarker is one or more immune cells.

16. The method of claim 12, wherein the biomarker is one or more of T− cells, NK cells and myeloid derived suppressor cells.

17. The method of any one of claims 1 to 6, wherein the biomarker is one or more of CD 4+ T cells, NK cells and β cells.

18. The method of any one of claims 1 to 17, wherein the fluid is obtained by density gradient centrifugation.

19. The method of any one of claims 1 to 18, wherein the volume of fluid is about 10 μL or more.

20. The method of any one of claims 1 to 19, wherein the fluid is blood plasma and the volume of the blood plasma is about 10 μL or more.

21. The method of any one of claims 1 to 20, wherein the fluid is buffy coat and the volume of the buffy coat is about 10 μL or more.

22. The method of any one of claims 1 to 21, wherein, after adding the fluid to the nanosensor, the fluid remains on the nanosensor for an incubation period.

23. The method of claim 22, wherein the incubation period is in a range of about 1 minute to about 2 minutes.

24. The method of any one of claims 1 to 23, wherein providing the cancer assessment includes providing a type of the cancer, a location of the cancer, a stage of the cancer, a metastatic potential of the cancer, or a therapy efficacy of the cancer.

25. The method of any one of claims 1 to 24, wherein providing the cancer assessment includes providing a prognosis for the patient.

26. The method of any one of claims 1 to 24 wherein providing the cancer assessment includes early cancer diagnosis.

27. The method of any one of claims 1 to 24 wherein providing the cancer assessment includes determining whether a tumor is benign or malignant.

28. The method of claim 27, wherein providing the cancer assessment includes, when the tumor is benign, determining weather the tumor has potential for malignancy.

29. The method of any one of claims 1 to 24, wherein providing the cancer assessment includes determining whether a tumor is primary or metastatic.

30. The method of any one of claims 1 to 24, wherein providing the cancer assessment includes determining whether a primary tumor has potential for metastasis.

31. The method of any one of claims 1 to 24, wherein providing the cancer assessment includes determining a progression of the cancer.

32. The method of any one of claims 1 to 24, wherein providing the cancer assessment includes determining a nodal metastasis of the cancer.

33. The method of any one of claims 1 to 24, wherein providing the cancer assessment includes determining a clinical metastasis of the cancer.

34. The method of any one of claims 1 to 24, wherein providing the cancer assessment includes determining a stage and/or grade of the cancer.

35. The method of any one of claims 1 to 24 wherein providing the cancer assessment includes a prediction of patient survival.

36. The method of any one of claims 1 to 24, wherein providing the cancer assessment includes providing a prognosis for the patient.

37. The method of any one of claims 1 to 23, wherein providing the cancer assessment includes providing an early diagnosis of cancer.

38. The method of claim 37, wherein providing the cancer assessment includes providing the early diagnosis of hard to detect cancers including brain cancer, ovarian cancer, kidney cancer, pancreatic cancer, liver cancer, lung cancer, or glastrointerstinal cancer.

39. The method of claim 38, wherein providing the cancer assessment includes determining a presence of an aggressive brain cancer including glioblastoma.

40. The method of any one of claims 1 to 24, wherein providing the cancer assessment includes providing a location of the tumor.

41. The method of any one of claims 1 to 24, wherein providing the cancer assessment includes determining a metastatic state of cancer to brain from a cancer site, the cancer site including lung tissue, breast tissue, colon tissue, kidney tissue, thyroid tissue and skin tissue.

42. The method of claim 41, wherein determining the metastatic state is by risk assessment based on a molecular phenotype of the tumor, the molecular phenotype including human epidermal growth factor receptor 2 (HER 2), epidermal growth factor receptor (EGFR) and/or isocitrate dehydrogenase (IDH).

43. The method of claim 42, wherein determining the metastatic state of cancer to brain from a cancer site includes determining the metastatic state of breast cancer based on a molecular phenotype of the tumor.

44. The method of claim 43, wherein the molecular phenotype of the tumor is HER2 positive or HER 2 negative.

45. The method of claim 42, wherein determining the metastatic state of cancer to brain from a cancer site includes determining the metastatic state of lung cancer based on a molecular phenotype of the tumor.

46. The method of claim 43, wherein the molecular phenotype of the tumor is EGFR.

47. The method of any one of claims 1 to 24, wherein providing the cancer assessment includes determining a metastatic state of cancer to localised metastasis or widespread from primary cancer sites.

48. The method of any one of claims 1 to 24, wherein providing the cancer assessment includes determining presence a gynaecological cancer, the gynaecological cancer being one of ovarian cancer, cervical cancer, or uterine cancer.

49. The method of any one of claims 1 to 24, wherein providing the cancer assessment includes monitoring cancer recurrence during or after therapy, the therapy including radiation therapy, immunotherapy and/or chemotherapy.

50. The method of any one of claims 1 to 24, wherein providing the cancer assessment includes monitoring cancer recurrence after surgery.

51. The method of any one of claims 1 to 24, wherein providing the cancer assessment includes determining a presence of minimal residual disease.

52. The method of claim 2, wherein the sample Raman spectrum includes second amplified signals indicating a presence of a second biomarker on the nanosensor and the method further comprises:
performing further data processing on the sample Raman spectrum to compare the second amplified signals to a second template Raman spectrum to determine a correlation between the sample Raman spectrum and the second template Raman spectrum, the template Raman spectrum being of a known cancer characteristic; and
based on both the correlation between the sample Raman spectrum and the template Raman spectrum and the second correlation between the sample Raman spectrum and the second template Raman spectrum, providing a diagnosis of the cancer in the patient.

53. A computing device for providing a cancer assessment for a patient for a sample that is a volume of a fluid sample from the patient that includes at least one biomarker; wherein the computing device comprises:
a data store comprising program instructions for obtaining Raman spectral data of the sample and performing cancer assessment of the sample using the sample Raman spectral data; and
a processing unit that is operatively coupled to the data store and when executing the program instructions is configured to:
acquire sample Raman spectral data from the fluid sample where the Raman spectral is obtained after adding at least a portion of the volume of the fluid sample to a nanosensor that comprises nanoparticles configured to capture the at least one biomarker, the sample Raman spectral data having amplified signals indicating the presence of the at least one biomarker on the nanosensor;
process the sample Raman spectral data using data from template Raman spectra from known cancer samples having cancer characteristics to detect whether the sample comprises one or more of the cancer characteristics; and
provide the cancer assessment of the patient based on the detected one or more cancer characteristics.

54. The computing device of claim 53, wherein the processing unit is further configured to perform the method of any one of claims 2 to 52.
